SEQUENCE LISTING (242 sequences)

SEQ ID NO: 1 - DNA, 6641 bp, Homo sapiens
gccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagtccaaaacttgaattctgggttgaattttttaaaaatcaggtaccacttgatttcatatgggaaattgaagcaggaaatattgagggcttcttgatcacagaaaactcagaagagatagtaatgctcaggacaggagcggcagccccagaacaggccactcatttagaattctagtgtttcaaaacacttttgtgtgttgtatggtcaataacatttttcattactgatggtgtcattcacccattaggtaaacattcccttttaaatgtttgtttgttttttgagacaggatctcactctgttgccagggctgtagtgcagtggtgtgatcatagctcactgcaacctccacctcccaggctcaagcctcccgaatagctgggactacaggcgcacaccaccatccccggctaatttttgtattttttgtagagacggggttttgccatgttgccaaggctggtttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggatacattttactaaaaggttcattggttccaatcacagctcataggtagagcaaagaaagggtggatggattgaaaagattagcctctgtctcggtggcaggttcccacctcgcaagcaattggaaacaaaacttttggggagttttattttgcattagggtgtgttttatgttaagcaaaacatactttagaaacaaatgaaaaaggcaattgaaaatcccagctatttcacctagatggaatagccaccctgagcagaactttgtgatgcttcattctgtggaattttgtgcttgctactgtatagtgcatgtggtgtaggttactctaactggttttgtcgacgtaaacatttaaagtgttatattttttataaaaatgtttatttttaatgatatgagaaaaattttgttaggccacaaaaacactgcactgtgaacattttagaaaaggtatgtcagactgggattaatgacagcatgattttcaatgactgtaaattgcgataaggaaatgtactgattgccaatacaccccaccctcattacatcatcaggacttgaagccaagggttaacccagcaagctacaaagagggtgtgtcacactgaaactcaatagttgagtttggctgttgttgcaggaaaatgattataactaaaagctctctgatagtgcagagacttaccagaagacacaaggaattgtactgaagagctattacaatccaaatattgccgtttcataaatgtaataagtaatactaattcacagagtattgtaaatggtggatgacaaaagaaaatctgctctgtggaaagaaagaactgtctctaccagggtcaagagcatgaacgcatcaatagaaagaactcggggaaacatcccatcaacaggactacacacttgtatatacattcttgagaacactgcaatgtgaaaatcacgtttgctatttataaacttgtccttagattaatgtgtctggacagattgtgggagtaagtgattcttctaagaattagatacttgtcactgcctatacctgcagctgaactgaatggtacttcgtatgttaatagttgttctgataaatcatgcaattaaagtaaagtgatgcaacatcttgtaaaaaaaaaaaaaaaaaaaa

SEQ ID NO: 2 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 3 - DNA, 1466 bp, Homo sapiens
ggcctgggtggcggtaaggtgtagctgttctgtcccagtccccgtagctctgcaccgagcagaagaggtctagggtcacgcaggggccatggctgaggcacttgggcttggttctggcaggcggagcggacggggctgagagcggggccctacgggcggaagaggagcactggaagaaggaagagacaaatgttggggtctacggattcagttccagtttaatgatgtggccctctaaggagataattttgccttaaacagaaagacttgaccctcttaggtgtcatacctaagatcagtgtgttttttgtgtccaattcttttatcaccaaaaaagagaagaaatattgcagtgaatgaagattcctctgcattttagcactgctttttcaactgtagttggcttttgaatgaggatgacaatggaagagatgaagaatgaagctgagaccacatccatggtttctatgcccctctatgcagtcatgtatcctgtgtttaatgagctagaacgagtaaatctgtctgcagcccagacactgagagccgctttcatcaaggctgaaaaagaaaatccaggtctcacacaagacatcattatgaaaattttagagaaaaaaagcgtggaagttaacttcacggagtcccttcttcgtatggcagctgatgatgtagaagagtatatgattgaacgaccagagccagaattccaagacctaaacgaaaaggcacgagcacttaaacaaattctcagtaagatcccagatgagatcaatgacagagtgaggtttctgcagacaatcaaggatatagctagtgcaataaaagaacttcttgatacagtgaataatgtcttcaagaaatatcaataccagaaccgcagggcacttgaacaccaaaagaaagaatttgtaaagtactccaaaagtttcagtgatactctgaaaacgtattttaaagatggcaaggcaataaatgtgttcgtaagtgccaaccgactaattcatcaaaccaacttaatacttcagaccttcaaaactgtggcctgaaagttgtatatgttaagagatgtacttctcagtggcagtattgaactgcctttatctgtaaattttaaagtttgactgtataaattatcagtccctcctgaagggatctaatccaggatgttgaatgggattattgccatcttacaccatatttttgtaaaatgtagcttaatcataatctcacactgaagattttgcatcacttttgctattatcattcttttaagaattataagccaaaagaatttacgccttaatgtgtcattatataacattccttaaaagaattgtaaatattggtgtttgtttctgacattttaacttgaaagcgatatgctgcaagataatgtatttaacaatatttggtggcaaatattcaataaatagtttacatctgttaaaaaaaaaaaaaaaaaa

SEQ ID NO: 4 - protein, 212 aa, Homo sapiens
MRMTMEEMKNEAETTSMVSMPLYAVMYPVFNELERVNLSAAQTLRAAFIKAEKENPGLTQDIIMKILEKKSVEVNFTESLLRMAADDVEEYMIERPEPEFQDLNEKARALKQILSKIPDEINDRVRFLQTIKDIASAIKELLDTVNNVFKKYQYQNRRALEHQKKEFVKYSKSFSDTLKTYFKDGKAINVFVSANRLIHQTNLILQTFKTVA

SEQ ID NO: 5 - DNA, 3317 bp, Homo sapiens
agttctcgcgggacaccgacggggagcggaagccaggaggtattgctgcttcggcgaccgggcggcggcagcggcggcggcggctgtggcagagtctgtgcctgtggcggtgacggcggcgggagcaagcgctgccctcgcagagcagccttggggtcgccggccgctcgcagcgttgtggaggggcgggccggacgctgagcggagcagctgcgccacgggtggcattgtgtgtcccagagtgccggagcgagtcccagaagagaggcgaggctaagcccagagcgctgggttgcttcagcagggaagactcccttccccctgcttcaggctgctgagcactgagcagcgctcagaatggaagccatcgccaaatatgacttcaaagctactgcagacgacgagctgagcttcaaaaggggggacatcctcaaggttttgaacgaagaatgtgatcagaactggtacaaggcagagcttaatggaaaagacggcttcattcccaagaactacatagaaatgaaaccacatccgtggttttttggcaaaatccccagagccaaggcagaagaaatgcttagcaaacagcggcacgatggggcctttcttatccgagagagtgagagcgctcctggggacttctccctctctgtcaagtttggaaacgatgtgcagcacttcaaggtgctccgagatggagccgggaagtacttcctctgggtggtgaagttcaattctttgaatgagctggtggattatcacagatctacatctgtctccagaaaccagcagatattcctgcgggacatagaacaggtgccacagcagccgacatacgtccaggccctctttgactttgatccccaggaggatggagagctgggcttccgccggggagattttatccatgtcatggataactcagaccccaactggtggaaaggagcttgccacgggcagaccggcatgtttccccgcaattatgtcacccccgtgaaccggaacgtctaagagtcaagaagcaattatttaaagaaagtgaaaaatgtaaaacacatacaaaagaattaaacccacaagctgcctctgacagcagcctgtgagggagtgcagaacacctggccgggtcaccctgtgaccctctcactttggttggaactttagggggtgggagggggcgttggatttaaaaatgccaaaacttacctataaattaagaagagtttttattacaaattttcactgctgctcctctttcccctcctttgtcttttttttcatccttttttctcttctgtccatcagtgcatgacgtttaaggccacgtatagtcctagctgacgccaataataaaaaacaagaaaccaagtgggctggtattctctctatgcaaaatgtctgttttagttggaatgactgaaagaagaacagctgttcctgtgttcttcgtatatacacacaaaaaggagcgggcagggccgctcgatgcctttgctgtttagcttcctccagaggaggggacttgtaggaatctgccttccagcccagacccccagtgtattttgtccaagttcacagtagagtagggtagaaggaaagcatgtctctgcttccatggcttcctgagaaagcccacctgggctgggcgcggtggctcacgcctgtaatcccagcactttgggaggccaaggtgggcggatcacaaggtcaggagttcgagaccaacctagccaacatggtgaaaccccgtctctactaaaaataagaaattagccgggtgtggcacgcacctgtagtcccagctacttgggagcctgaggcaggagaatcgcttgaacctgggaagtggaggttgagtgagccgggaccgtgccattgtactccagcctgggtgacagagcgagattccgtctcaaaaaaaaaaaaaaaaagcccacctgaaagcctgtctctttccactttgttggcccttccagtgggattatcgagcatgttgttttttcatagtgcctttttccttatttcaagggttgcttctgagtggtgttttttttttttttttaatttgttttgttttaaaataagttaaaggcagtccagagcttttcagccaatttgtctcctactctgtgtaaatatttttccctccgggcaggggagccagggtagagcaaaggagacaaagcaggagtggaaggtgaggcgttctcctgcttgtactaagccaggaggctttaagctccagctttaagggttgtgagccccttgggggttcagggaactgcttgcccagggtgcagtgtgagtgtgatgggccaccggggcaagagggaaggtgaccgcccagctctcccacatcccactggatctggcttacaggggggtcggaagcctgtcctcaccgtctcgggggttgtggcccccgccccctccctatatgcacccctggaaccagcaagtcccagacaaggagagcggaggaggaagtcatgggaacgcagcctccagttgtagcaggtttcactattcctatgctggggtacacagtgagagtactcacttttcacttgtcttgctcttagattgggccatggctttcatcctgtgtcccctgacctgtccaggtgagtgtgagggcagcactgggaagctggagtgctgcttgtgcctcccttcccagtgggctgtgttgactgctgctccccacccctaccgatggtcccaggaagcagggagagttggggaaggcaagattggaaagacaggaagaccaaggcctcggcagaactctctgtcttctctccacttctggtcccctgtggtgatgtgcctgtaatctttttctccacccaaaccccttcccacgacaaaaacaagactgcctccctctcttccgggagctggtgacagccttgggcctttcagtcccaaagcggccgatgggagtctccctccgactccagatatgaacagggcccaggcctggagcgtttgctgtgccaggaggcggcagctcttctgggcagagcctgtccccgccttccctcactcttcctcatcctgcttctcttttcctcgcagatgataaaaggaatctggcattctacacctggaccatttgattgttttattttggaattggtgtatatcatgaagccttgctgaactaagttttgtgtgtatatatttaaaaaaaaaatcagtgtttaaataaagacctatgtacttaatcctttaactctgcggatagcatttggtaggtagtgattaactgtgaataataaatacacaatgaattcttcaaaaaaaaaaaaaaa

SEQ ID NO: 6 - protein, 217 aa, Homo sapiens [sequence omitted]

SEQ ID NO: 7 - DNA, 21 bp, Artificial Sequence
  Homo sapiens siRNA sequence
aacacccatccagaatgtcat

SEQ ID NO: 8 - DNA, 21 bp, Artificial Sequence
  Homo sapiens siRNA sequence
aagccaatttatcaggaggtg

SEQ ID NO: 9 - DNA, 21 bp, Artificial Sequence
  Homo sapiens siRNA sequence
tcaagagcatgaacgcatcaa

SEQ ID NO: 10 - DNA, 21 bp, Artificial Sequence
  Homo sapiens siRNA sequence
tgtgtgttgtatggtcaataa

SEQ ID NO: 11 - DNA, 21 bp, Artificial Sequence
  Homo sapiens siRNA sequence
actgaatggtacttcgtatgt

SEQ ID NO: 12 - DNA, 21 bp, Artificial Sequence
  Homo sapiens siRNA sequence
cctcgcaagcaattggaaaca

SEQ ID NO: 13 - DNA, 21 bp, Artificial Sequence
  Homo sapiens siRNA sequence
ctctgatagtgcagagactta

SEQ ID NO: 14 - DNA, 21 bp, Artificial Sequence
  Homo sapiens siRNA sequence
gaatgatgctactctgatcta

SEQ ID NO: 15 - DNA, 21 bp, Artificial Sequence
  Homo sapiens siRNA sequence
gcaatacagtcaaagtttcaa

SEQ ID NO: 16 - DNA, 21 bp, Artificial Sequence
  Homo sapiens siRNA sequence
ttgtgtgttgtatggtcaata

SEQ ID NO: 17 - DNA, 21 bp, Artificial Sequence
  Homo sapiens siRNA sequence
tttgtgtgttgtatggtcaat

SEQ ID NO: 18 - DNA, 21 bp, Artificial Sequence
  Homo sapiens siRNA sequence
aggactacacacttgtatata

SEQ ID NO: 19 - DNA, 21 bp, Artificial Sequence
  Homo sapiens siRNA sequence
agagtattgtaaatggtggat

SEQ ID NO: 20 - DNA, 21 bp, Artificial Sequence
  Homo sapiens siRNA sequence
gaatggtacttcgtatgttaa

SEQ ID NO: 21 - DNA, 21 bp, Artificial Sequence
  Homo sapiens siRNA sequence
ctgtaaattgcgataaggaaa

SEQ ID NO: 22 - DNA, 21 bp, Artificial Sequence
  Homo sapiens siRNA sequence
ccaaatattgccgtttcataa

SEQ ID NO: 23 - DNA, 21 bp, Artificial Sequence
  Homo sapiens siRNA sequence
caagagcatgaacgcatcaat

SEQ ID NO: 24 - DNA, 21 bp, Artificial Sequence
  Homo sapiens siRNA sequence
gcatgaacgcatcaatagaaa

SEQ ID NO: 25 - DNA, 21 bp, Artificial Sequence
  Homo sapiens siRNA sequence
gaagagctattacaatccaaa

SEQ ID NO: 26 - DNA, 21 bp, Artificial Sequence
  Homo sapiens siRNA sequence
cattacatcatcaggacttga

SEQ ID NO: 27 - DNA, 21 bp, Artificial Sequence
  Homo sapiens siRNA sequence
acaggactacacacttgtata

SEQ ID NO: 28 - DNA, 21 bp, Artificial Sequence
  Homo sapiens siRNA sequence
caggactacacacttgtatat

SEQ ID NO: 29 - DNA, 21 bp, Artificial Sequence
  Homo sapiens siRNA sequence
aatctgtctgcagcccagaac

SEQ ID NO: 30 - DNA, 21 bp, Artificial Sequence
  Homo sapiens siRNA sequence
aagcgtggaagttaacttcac

SEQ ID NO: 31 - DNA, 21 bp, Artificial Sequence
  Homo sapiens siRNA sequence
agtcatgtatcctgtgtttaa

SEQ ID NO: 32 - DNA, 21 bp, Artificial Sequence
  Homo sapiens siRNA sequence
ggatatagctagtgcaataaa

SEQ ID NO: 33 - DNA, 21 bp, Artificial Sequence
  Homo sapiens siRNA sequence
acgccttaatgtgtcattata

SEQ ID NO: 34 - DNA, 21 bp, Artificial Sequence
  Homo sapiens siRNA sequence
ttaaagatggcaaggcaataa

SEQ ID NO: 35 - DNA, 21 bp, Artificial Sequence
  Homo sapiens siRNA sequence
ccgctttcatcaaggctgaaa

SEQ ID NO: 36 - DNA, 21 bp, Artificial Sequence
  Homo sapiens siRNA sequence
tcgtaagtgccaaccgactaa

SEQ ID NO: 37 - DNA, 21 bp, Artificial Sequence
  Homo sapiens siRNA sequence
agcgatatgctgcaagataat

SEQ ID NO: 38 - DNA, 21 bp, Artificial Sequence
  Homo sapiens siRNA sequence
acttaatacttcagaccttca

SEQ ID NO: 39 - DNA, 21 bp, Artificial Sequence
  Homo sapiens siRNA sequence
cagtcatgtatcctgtgttta

SEQ ID NO: 40 - DNA, 21 bp, Artificial Sequence
  Homo sapiens siRNA sequence
cttaatacttcagaccttcaa

SEQ ID NO: 41 - DNA, 21 bp, Artificial Sequence
  Homo sapiens siRNA sequence
ctgatgatgtagaagagtata

SEQ ID NO: 42 - DNA, 21 bp, Artificial Sequence
  Homo sapiens siRNA sequence
tgaactgcctttatctgtaaa

SEQ ID NO: 43 - DNA, 21 bp, Artificial Sequence
  Homo sapiens siRNA sequence
acggattcagttccagtttaa

SEQ ID NO: 44 - DNA, 21 bp, Artificial Sequence
  Homo sapiens siRNA sequence
tttaaagatggcaaggcaata

SEQ ID NO: 45 - DNA, 21 bp, Artificial Sequence
  Homo sapiens siRNA sequence
ttaatgagctagaacgagtaa

SEQ ID NO: 46 - DNA, 21 bp, Artificial Sequence
  Homo sapiens siRNA sequence
tgaaagcgatatgctgcaaga

SEQ ID NO: 47 - DNA, 21 bp, Artificial Sequence
  Homo sapiens siRNA sequence
gaaagcgatatgctgcaagat

SEQ ID NO: 48 - DNA, 21 bp, Artificial Sequence
  Homo sapiens siRNA sequence
cttgaaagcgatatgctgcaa

SEQ ID NO: 49 - DNA, 21 bp, Artificial Sequence
  Homo sapiens siRNA sequence
tcataatctcacactgaagat

SEQ ID NO: 50 - DNA, 21 bp, Artificial Sequence
  Homo sapiens siRNA sequence
tattgccatcttacaccatat

SEQ ID NO: 51 - DNA, 21 bp, Artificial Sequence
  Homo sapiens siRNA sequence
aatccccagagccaaggcaga

SEQ ID NO: 52 - DNA, 21 bp, Artificial Sequence
  Homo sapiens siRNA sequence
aaggggggacatcctcaaggt

SEQ ID NO: 53 - DNA, 21 bp, Artificial Sequence
  Homo sapiens siRNA sequence
agtcctagctgacgccaataa

SEQ ID NO: 54 - DNA, 21 bp, Artificial Sequence
  Homo sapiens siRNA sequence
ggtagtgattaactgtgaata

SEQ ID NO: 55 - DNA, 21 bp, Artificial Sequence
  Homo sapiens siRNA sequence
ctccagttgtagcaggtttca

SEQ ID NO: 56 - DNA, 21 bp, Artificial Sequence
  Homo sapiens siRNA sequence
ttcctgtgttcttcgtatata

SEQ ID NO: 57 - DNA, 21 bp, Artificial Sequence
  Homo sapiens siRNA sequence
tccatcagtgcatgacgttta

SEQ ID NO: 58 - DNA, 21 bp, Artificial Sequence
  Homo sapiens siRNA sequence
cctgtggtgatgtgcctgtaa

SEQ ID NO: 59 - DNA, 21 bp, Artificial Sequence
  Homo sapiens siRNA sequence
ggaacgtctaagagtcaagaa

SEQ ID NO: 60 - DNA, 21 bp, Artificial Sequence
  Homo sapiens siRNA sequence
agaagaaatgcttagcaaaca

SEQ ID NO: 61 - DNA, 21 bp, Artificial Sequence
  Homo sapiens siRNA sequence
tagtcctagctgacgccaata

SEQ ID NO: 62 - DNA, 21 bp, Artificial Sequence
  Homo sapiens siRNA sequence
tgacgtttaaggccacgtata

SEQ ID NO: 63 - DNA, 21 bp, Artificial Sequence
  Homo sapiens siRNA sequence
catgaagccttgctgaactaa

SEQ ID NO: 64 - DNA, 21 bp, Artificial Sequence
  Homo sapiens siRNA sequence
gtctccagaaaccagcagata

SEQ ID NO: 65 - DNA, 21 bp, Artificial Sequence
  Homo sapiens siRNA sequence
gttcctgtgttcttcgtatat

SEQ ID NO: 66 - DNA, 21 bp, Artificial Sequence
  Homo sapiens siRNA sequence
catttggtaggtagtgattaa

SEQ ID NO: 67 - DNA, 21 bp, Artificial Sequence
  Homo sapiens siRNA sequence
gctcgatgcctttgctgttta

SEQ ID NO: 68 - DNA, 21 bp, Artificial Sequence
  Homo sapiens siRNA sequence
ctgtggtgatgtgcctgtaat

SEQ ID NO: 69 - DNA, 21 bp, Artificial Sequence
  Homo sapiens siRNA sequence
gcatttggtaggtagtgatta

SEQ ID NO: 70 - DNA, 21 bp, Artificial Sequence
  Homo sapiens siRNA sequence
tcagccaatttgtctcctact

SEQ ID NO: 71 - DNA, 21 bp, Artificial Sequence
  Homo sapiens siRNA sequence
atatcatgaagccttgctgaa

SEQ ID NO: 72 - DNA, 21 bp, Artificial Sequence
  Homo sapiens siRNA sequence
tactaagccaggaggctttaa

SEQ ID NO: 73 - DNA, 21 bp, Artificial Sequence
  Homo sapiens siRNA sequence
ucugucugcagcccagacatt

SEQ ID NO: 74 - DNA, 21 bp, Artificial Sequence
  Homo sapiens siRNA sequence
ugucugggcugcagacagatt

SEQ ID NO: 75 - DNA, 21 bp, Artificial Sequence
  Homo sapiens siRNA sequence
gcguggaaguuaacuucactt

SEQ ID NO: 76 - DNA, 21 bp, Artificial Sequence
  Homo sapiens siRNA sequence
gugaaguuaacuuccacgctt

SEQ ID NO: 77 - DNA, 21 bp, Artificial Sequence
  Homo sapiens siRNA sequence
ucgagacccugguggacautt

SEQ ID NO: 78 - DNA, 21 bp, Artificial Sequence
  Homo sapiens siRNA sequence
auguccaccagggucucgatt

SEQ ID NO: 79 - DNA, 21 bp, Artificial Sequence
  Homo sapiens siRNA sequence
ggccagcacauaggagagatt

SEQ ID NO: 80 - DNA, 21 bp, Artificial Sequence
  Homo sapiens siRNA sequence
ucucuccuaugugcuggcctt

SEQ ID NO: 81 - DNA, 21 bp, Artificial Sequence
  Homo sapiens siRNA sequence
cacccauccagaaugucautt

SEQ ID NO: 82 - DNA, 21 bp, Artificial Sequence
  Homo sapiens siRNA sequence
augacauucuggaugggugtt

SEQ ID NO: 83 - DNA, 21 bp, Artificial Sequence
  Homo sapiens siRNA sequence
gccaauuuaucaggaggugtt

SEQ ID NO: 84 - DNA, 21 bp, Artificial Sequence
  Homo sapiens siRNA sequence
caccuccugauaaauuggctt

SEQ ID NO: 85 - DNA, 21 bp, Artificial Sequence
  Green fluorescent protein siRNA sequence
gctgaccctgaagttcatctt

SEQ ID NO: 86 - DNA, 21 bp, Artificial Sequence
  Green fluorescent protein siRNA sequence
gaugaacuucagggucagctt

SEQ ID NO: 87 - DNA, 21 bp, Artificial Sequence
  Green fluorescent protein siRNA sequence
gcagcacgacuucuucaagtt

SEQ ID NO: 88 - DNA, 21 bp, Artificial Sequence
  Green fluorescent protein siRNA sequence
cuugaagaagucgugcugctt

SEQ ID NO: 89 - DNA, 21 bp, Artificial Sequence
  Homo sapiens siRNA sequence
ggggggacauccucaaggutt

SEQ ID NO: 90 - DNA, 21 bp, Artificial Sequence
  Homo sapiens siRNA sequence
accuugaggaugucccccctt

SEQ ID NO: 91 - DNA, 21 bp, Artificial Sequence
  Homo sapiens siRNA sequence
uccccagagccaaggcagatt

SEQ ID NO: 92 - DNA, 21 bp, Artificial Sequence
  Homo sapiens siRNA sequence
ucugccuuggcucuggggatt

SEQ ID NO: 93 - DNA, 21 bp, Artificial Sequence
  Homo sapiens siRNA sequence
gaguuaccuuccuaaugcatt

SEQ ID NO: 94 - DNA, 21 bp, Artificial Sequence
  Homo sapiens siRNA sequence
ugcauuaggaagguaacuctt

SEQ ID NO: 95 - DNA, 21 bp, Artificial Sequence
  Homo sapiens siRNA sequence
gauucccuugguauauucatt

SEQ ID NO: 96 - DNA, 21 bp, Artificial Sequence
  Homo sapiens siRNA sequence
ugaauauaccaagggaauctt

SEQ ID NO: 97 - DNA, 21 bp, Artificial Sequence
  Homo sapiens siRNA sequence
gacaggaguggagugauautt

SEQ ID NO: 98 - DNA, 21 bp, Artificial Sequence
  Homo sapiens siRNA sequence
auaucacuccacuccuguctt

SEQ ID NO: 99 - DNA, 21 bp, Artificial Sequence
  Homo sapiens siRNA sequence
cuucagcgaguucacgggutt

SEQ ID NO: 100 - DNA, 21 bp, Artificial Sequence
  Homo sapiens siRNA sequence
acccgugaacucgcugaagtt

SEQ ID NO: 101 - DNA, 21 bp, Artificial Sequence
  Homo sapiens siRNA sequence
uggguccuuucuuuggacutt

SEQ ID NO: 102 - DNA, 21 bp, Artificial Sequence
  Homo sapiens siRNA sequence
aguccaaagaaaggacccatt

SEQ ID NO: 103 - DNA, 21 bp, Artificial Sequence
  Homo sapiens siRNA sequence
cugcuugaagcagcucuggtt

SEQ ID NO: 104 - DNA, 21 bp, Artificial Sequence
  Homo sapiens siRNA sequence
ccagagcugcuucaagcagtt

SEQ ID NO: 105 - DNA, 21 bp, Artificial Sequence
  Homo sapiens siRNA sequence
gcucagaagcaguauugggtt

SEQ ID NO: 106 - DNA, 21 bp, Artificial Sequence
  Homo sapiens siRNA sequence
cccaauacugcuucugagctt

SEQ ID NO: 107 - DNA, 21 bp, Artificial Sequence
  Homo sapiens siRNA sequence
ugcaauacccaauuucaautt

SEQ ID NO: 108 - DNA, 21 bp, Artificial Sequence
  Homo sapiens siRNA sequence
auugaaauuggguauugcatt

SEQ ID NO: 109 - DNA, 21 bp, Artificial Sequence
  Homo sapiens siRNA sequence
ugauugggccuaugcggcctt

SEQ ID NO: 110 - DNA, 21 bp, Artificial Sequence
  Homo sapiens siRNA sequence
ggccgcauaggcccaaucatt

SEQ ID NO: 111 - DNA, 21 bp, Artificial Sequence
  Homo sapiens siRNA sequence
guggaaccagcaacaccugtt

SEQ ID NO: 112 - DNA, 21 bp, Artificial Sequence
  Homo sapiens siRNA sequence
cagguguugcugguuccactt

SEQ ID NO: 113 - DNA, 19 bp, Artificial Sequence
  Homo sapiens siRNA sequence
ggcagctgatgatgtagaa

SEQ ID NO: 114 - DNA, 19 bp, Artificial Sequence
  Homo sapiens siRNA sequence
gccagaattccaagaccta

SEQ ID NO: 115 - DNA, 19 bp, Artificial Sequence
  Homo sapiens siRNA sequence
ccagaattccaagacctaa

SEQ ID NO: 116 - DNA, 19 bp, Artificial Sequence
  Homo sapiens siRNA sequence
ggcacgagcacttaaacaa

SEQ ID NO: 117 - DNA, 19 bp, Artificial Sequence
  Homo sapiens siRNA sequence
gcacgagcacttaaacaaa

SEQ ID NO: 118 - DNA, 19 bp, Artificial Sequence
  Homo sapiens siRNA sequence
ggtttctgcagacaatcaa

SEQ ID NO: 119 - DNA, 19 bp, Artificial Sequence
  Homo sapiens siRNA sequence
gcagacaatcaaggatata

SEQ ID NO: 120 - DNA, 19 bp, Artificial Sequence
  Homo sapiens siRNA sequence
cctcctgaagggatctaat

SEQ ID NO: 121 - DNA, 19 bp, Artificial Sequence
  Homo sapiens siRNA sequence
ggatctaatccaggatgtt

SEQ ID NO: 122 - DNA, 19 bp, Artificial Sequence
  Homo sapiens siRNA sequence
ggatgttgaatgggattat

SEQ ID NO: 123 - DNA, 25 bp, Artificial Sequence
  Homo sapiens siRNA sequence
ccttcttcgtatggcagctgatgat

SEQ ID NO: 124 - DNA, 25 bp, Artificial Sequence
  Homo sapiens siRNA sequence
cagagccagaattccaagacctaaa

SEQ ID NO: 125 - DNA, 25 bp, Artificial Sequence
  Homo sapiens siRNA sequence
ccagaattccaagacctaaacgaaa

SEQ ID NO: 126 - DNA, 25 bp, Artificial Sequence
  Homo sapiens siRNA sequence
gacaatcaaggatatagctagtgca

SEQ ID NO: 127 - DNA, 25 bp, Artificial Sequence
  Homo sapiens siRNA sequence
ggcaaggcaataaatgtgttcgtaa

SEQ ID NO: 128 - DNA, 25 bp, Artificial Sequence
  Homo sapiens siRNA sequence
tcgtaagtgccaaccgactaattca

SEQ ID NO: 129 - DNA, 25 bp, Artificial Sequence
  Homo sapiens siRNA sequence
ccgactaattcatcaaaccaactta

SEQ ID NO: 130 - DNA, 25 bp, Artificial Sequence
  Homo sapiens siRNA sequence
tcagtccctcctgaagggatctaat

SEQ ID NO: 131 - DNA, 25 bp, Artificial Sequence
  Homo sapiens siRNA sequence
gaagggatctaatccaggatgttga

SEQ ID NO: 132 - DNA, 25 bp, Artificial Sequence
  Homo sapiens siRNA sequence
gggattattgccatcttacaccata

SEQ ID NO: 133 - DNA, 19 bp, Artificial Sequence
  Homo sapiens siRNA sequence
ccggttcatcaacttcttt

SEQ ID NO: 134 - DNA, 19 bp, Artificial Sequence
  Homo sapiens siRNA sequence
ggaccagtcctacattgat

SEQ ID NO: 135 - DNA, 19 bp, Artificial Sequence
  Homo sapiens siRNA sequence
gcacaaagcaagccagatt

SEQ ID NO: 136 - DNA, 19 bp, Artificial Sequence
  Homo sapiens siRNA sequence
gcatgtcaacatcgctcta

SEQ ID NO: 137 - DNA, 19 bp, Artificial Sequence
  Homo sapiens siRNA sequence
gctggtgttgtctcaatat

SEQ ID NO: 138 - DNA, 19 bp, Artificial Sequence
  Homo sapiens siRNA sequence
ggtgttgtctcaatatcaa

SEQ ID NO: 139 - DNA, 19 bp, Artificial Sequence
  Homo sapiens siRNA sequence
gcagtgaattagttcgcta

SEQ ID NO: 140 - DNA, 19 bp, Artificial Sequence
  Homo sapiens siRNA sequence
ccaactacagaaatggttt

SEQ ID NO: 141 - DNA, 19 bp, Artificial Sequence
  Homo sapiens siRNA sequence
ccatgtgaacgctacttat

SEQ ID NO: 142 - DNA, 19 bp, Artificial Sequence
  Homo sapiens siRNA sequence
gcatcagaaccagaggctt

SEQ ID NO: 143 - DNA, 25 bp, Artificial Sequence
  Homo sapiens siRNA sequence
caaagccaatttatcaggaggtgtt

SEQ ID NO: 144 - DNA, 25 bp, Artificial Sequence
  Homo sapiens siRNA sequence
cagtcggaggttcactgcatattct

SEQ ID NO: 145 - DNA, 25 bp, Artificial Sequence
  Homo sapiens siRNA sequence
cacacaagaataatcaggttctgtt

SEQ ID NO: 146 - DNA, 25 bp, Artificial Sequence
  Homo sapiens siRNA sequence
cgctctaattcagagataatctgtt

SEQ ID NO: 147 - DNA, 25 bp, Artificial Sequence
  Homo sapiens siRNA sequence
cagcactgttattactacttgggtt

SEQ ID NO: 148 - DNA, 25 bp, Artificial Sequence
  Homo sapiens siRNA sequence
ccagtagcctgattgtgcatttcaa

SEQ ID NO: 149 - DNA, 25 bp, Artificial Sequence
  Homo sapiens siRNA sequence
cagcctccttctgggagacatcata

SEQ ID NO: 150 - DNA, 25 bp, Artificial Sequence
  Homo sapiens siRNA sequence
tgggagacatcatagtgctagtact

SEQ ID NO: 151 - DNA, 25 bp, Artificial Sequence
  Homo sapiens siRNA sequence
gcaggaaatattgagggcttcttga

SEQ ID NO: 152 - DNA, 25 bp, Artificial Sequence
  Homo sapiens siRNA sequence
gccactcatttagaattctagtgtt

SEQ ID NO: 153 - DNA, 19 bp, Artificial Sequence
  Homo sapiens siRNA sequence
ccggttcatcaacttcttt

SEQ ID NO: 154 - DNA, 19 bp, Artificial Sequence
  Homo sapiens siRNA sequence
ggaccagtcctacattgat

SEQ ID NO: 155 - DNA, 19 bp, Artificial Sequence
  Homo sapiens siRNA sequence
gcacaaagcaagccagatt

SEQ ID NO: 156 - DNA, 19 bp, Artificial Sequence
  Homo sapiens siRNA sequence
gcatgtcaacatcgctcta

SEQ ID NO: 157 - DNA, 19 bp, Artificial Sequence
  Homo sapiens siRNA sequence
gctggtgttgtctcaatat

SEQ ID NO: 158 - DNA, 19 bp, Artificial Sequence
  Homo sapiens siRNA sequence
ggtgttgtctcaatatcaa

SEQ ID NO: 159 - DNA, 19 bp, Artificial Sequence
  Homo sapiens siRNA sequence
gcagtgaattagttcgcta

SEQ ID NO: 160 - DNA, 19 bp, Artificial Sequence
  Homo sapiens siRNA sequence
ccaactacagaaatggttt

SEQ ID NO: 161 - DNA, 19 bp, Artificial Sequence
  Homo sapiens siRNA sequence
ccatgtgaacgctacttat

SEQ ID NO: 162 - DNA, 19 bp, Artificial Sequence
  Homo sapiens siRNA sequence
gcatcagaaccagaggctt

SEQ ID NO: 163 - DNA, 25 bp, Artificial Sequence
  Homo sapiens siRNA sequence
caaagccaatttatcaggaggtgtt

SEQ ID NO: 164 - DNA, 25 bp, Artificial Sequence
  Homo sapiens siRNA sequence
cagtcggaggttcactgcatattct

SEQ ID NO: 165 - DNA, 25 bp, Artificial Sequence
  Homo sapiens siRNA sequence
cacacaagaataatcaggttctgtt

SEQ ID NO: 166 - DNA, 25 bp, Artificial Sequence
  Homo sapiens siRNA sequence
cgctctaattcagagataatctgtt

SEQ ID NO: 167 - DNA, 25 bp, Artificial Sequence
  Homo sapiens siRNA sequence
cagcactgttattactacttgggtt

SEQ ID NO: 168 - DNA, 25 bp, Artificial Sequence
  Homo sapiens siRNA sequence
ccagtagcctgattgtgcatttcaa

SEQ ID NO: 169 - DNA, 25 bp, Artificial Sequence
  Homo sapiens siRNA sequence
cagcctccttctgggagacatcata

SEQ ID NO: 170 - DNA, 25 bp, Artificial Sequence
  Homo sapiens siRNA sequence
tgggagacatcatagtgctagtact

SEQ ID NO: 171 - DNA, 25 bp, Artificial Sequence
  Homo sapiens siRNA sequence
gcaggaaatattgagggcttcttga

SEQ ID NO: 172 - DNA, 25 bp, Artificial Sequence
  Homo sapiens siRNA sequence
gccactcatttagaattctagtgtt

SEQ ID NO: 173 - DNA, 19 bp, Artificial Sequence
  Homo sapiens siRNA sequence
ggactctgtgaggaaacaa

SEQ ID NO: 174 - DNA, 19 bp, Artificial Sequence
  Homo sapiens siRNA sequence
gcttccagtcagacgtcta

SEQ ID NO: 175 - DNA, 19 bp, Artificial Sequence
  Homo sapiens siRNA sequence
ccaccagccaatcaatgtt

SEQ ID NO: 176 - DNA, 19 bp, Artificial Sequence
  Homo sapiens siRNA sequence
ccaatcaatgttcgtctct

SEQ ID NO: 177 - DNA, 19 bp, Artificial Sequence
  Homo sapiens siRNA sequence
tctccaatggctgggattt

SEQ ID NO: 178 - DNA, 19 bp, Artificial Sequence
  Homo sapiens siRNA sequence
gggatttgtggcagggatt

SEQ ID NO: 179 - DNA, 19 bp, Artificial Sequence
  Homo sapiens siRNA sequence
gcagggattccactcagaa

SEQ ID NO: 180 - DNA, 19 bp, Artificial Sequence
  Homo sapiens siRNA sequence
gccattcaaggactcctct

SEQ ID NO: 181 - DNA, 19 bp, Artificial Sequence
  Homo sapiens siRNA sequence
tcctctctttcttcaccaa

SEQ ID NO: 182 - DNA, 19 bp, Artificial Sequence
  Homo sapiens siRNA sequence
tctctttcttcaccaagaa

SEQ ID NO: 183 - DNA, 25 bp, Artificial Sequence
  Homo sapiens siRNA sequence
ggcggactctgtgaggaaacaagaa

SEQ ID NO: 184 - DNA, 25 bp, Artificial Sequence
  Homo sapiens siRNA sequence
gggttgtgctctacgagcttatgac

SEQ ID NO: 185 - DNA, 25 bp, Artificial Sequence
  Homo sapiens siRNA sequence
gctctacgagcttatgactggctca

SEQ ID NO: 186 - DNA, 25 bp, Artificial Sequence
  Homo sapiens siRNA sequence
cacaattgagctgctgcaacggtca

SEQ ID NO: 187 - DNA, 25 bp, Artificial Sequence
  Homo sapiens siRNA sequence
ccttgcccaccagccaatcaatgtt

SEQ ID NO: 188 - DNA, 25 bp, Artificial Sequence
  Homo sapiens siRNA sequence
accagccaatcaatgttcgtctctg

SEQ ID NO: 189 - DNA, 25 bp, Artificial Sequence
  Homo sapiens siRNA sequence
ccatctccaatggctgggatttgtg

SEQ ID NO: 190 - DNA, 25 bp, Artificial Sequence
  Homo sapiens siRNA sequence
ccgccattcaaggactcctctcttt

SEQ ID NO: 191 - DNA, 25 bp, Artificial Sequence
  Homo sapiens siRNA sequence
gccattcaaggactcctctctttct

SEQ ID NO: 192 - DNA, 25 bp, Artificial Sequence
  Homo sapiens siRNA sequence
ggactcctctctttcttcaccaaga

SEQ ID NO: 193 - DNA, 19 bp, Artificial Sequence
  Homo sapiens siRNA sequence
ggcctgagaggtctctcgt

SEQ ID NO: 194 - DNA, 19 bp, Artificial Sequence
  Homo sapiens siRNA sequence
gcctgagaggtctctcgtc

SEQ ID NO: 195 - DNA, 19 bp, Artificial Sequence
  Homo sapiens siRNA sequence
gagaggtctctcgtcgctg

SEQ ID NO: 196 - DNA, 19 bp, Artificial Sequence
  Homo sapiens siRNA sequence
cccatggccgcctactctt

SEQ ID NO: 197 - DNA, 19 bp, Artificial Sequence
  Homo sapiens siRNA sequence
ccatggccgcctactctta

SEQ ID NO: 198 - DNA, 19 bp, Artificial Sequence
  Homo sapiens siRNA sequence
gctctcagggaggtctgtg

SEQ ID NO: 199 - DNA, 25 bp, Artificial Sequence
  Homo sapiens siRNA sequence
ggagtcggcctgagaggtctctcgt

SEQ ID NO: 200 - DNA, 25 bp, Artificial Sequence
  Homo sapiens siRNA sequence
gagtcggcctgagaggtctctcgtc

SEQ ID NO: 201 - DNA, 25 bp, Artificial Sequence
  Homo sapiens siRNA sequence
tcggcctgagaggtctctcgtcgct

SEQ ID NO: 202 - DNA, 25 bp, Artificial Sequence
  Homo sapiens siRNA sequence
ccttggcccatggccgcctactctt

SEQ ID NO: 203 - DNA, 19 bp, Artificial Sequence
  Homo sapiens siRNA sequence
gctcgaaatcttacgcaaa

SEQ ID NO: 204 - DNA, 19 bp, Artificial Sequence
  Homo sapiens siRNA sequence
gcttatatcagtagcaatt

SEQ ID NO: 205 - DNA, 19 bp, Artificial Sequence
  Homo sapiens siRNA sequence
gcacccatctctaattata

SEQ ID NO: 206 - DNA, 19 bp, Artificial Sequence
  Homo sapiens siRNA sequence
gcactagaatttaaaccta

SEQ ID NO: 207 - DNA, 19 bp, Artificial Sequence
  Homo sapiens siRNA sequence
gccgttatcattccaagat

SEQ ID NO: 208 - DNA, 19 bp, Artificial Sequence
  Homo sapiens siRNA sequence
ccacacatcttcaagactt

SEQ ID NO: 209 - DNA, 19 bp, Artificial Sequence
  Homo sapiens siRNA sequence
gcacatcaaggtgctaata

SEQ ID NO: 210 - DNA, 19 bp, Artificial Sequence
  Homo sapiens siRNA sequence
gcaattaatggtctttctt

SEQ ID NO: 211 - DNA, 19 bp, Artificial Sequence
  Homo sapiens siRNA sequence
gcagttatgatttagctaa

SEQ ID NO: 212 - DNA, 19 bp, Artificial Sequence
  Homo sapiens siRNA sequence
gcaaccaactacctcatat

SEQ ID NO: 213 - DNA, 25 bp, Artificial Sequence
  Homo sapiens siRNA sequence
cctgaaatttgtaactcctaaagta

SEQ ID NO: 214 - DNA, 25 bp, Artificial Sequence
  Homo sapiens siRNA sequence
gcagttgtcttaaacagattgataa

SEQ ID NO: 215 - DNA, 25 bp, Artificial Sequence
  Homo sapiens siRNA sequence
caacctgcttattgcaacaagtatt

SEQ ID NO: 216 - DNA, 25 bp, Artificial Sequence
  Homo sapiens siRNA sequence
catcaatagatactgtgctagatta

SEQ ID NO: 217 - DNA, 25 bp, Artificial Sequence
  Homo sapiens siRNA sequence
tccagagtgtttgagggatagttat

SEQ ID NO: 218 - DNA, 25 bp, Artificial Sequence
  Homo sapiens siRNA sequence
cctttacctgatgaactcaacttta

SEQ ID NO: 219 - DNA, 25 bp, Artificial Sequence
  Homo sapiens siRNA sequence
cagcatactgtgttctacctcttaa

SEQ ID NO: 220 - DNA, 25 bp, Artificial Sequence
  Homo sapiens siRNA sequence
ccaaatgggaaagtctgcagaataa

SEQ ID NO: 221 - DNA, 25 bp, Artificial Sequence
  Homo sapiens siRNA sequence
cagccgcatggtggtgtcaatattt

SEQ ID NO: 222 - DNA, 25 bp, Artificial Sequence
  Homo sapiens siRNA sequence
ccgcatggtggtgtcaatatttgat

SEQ ID NO: 223 - DNA, 19 bp, Artificial Sequence
  Homo sapiens siRNA sequence
gcaatacccaatttcaatt

SEQ ID NO: 224 - DNA, 19 bp, Artificial Sequence
  Homo sapiens siRNA sequence
gaatcttccaaagcgcaaa

SEQ ID NO: 225 - DNA, 19 bp, Artificial Sequence
  Homo sapiens siRNA sequence
tcttccaaagcgcaaagaa

SEQ ID NO: 226 - DNA, 19 bp, Artificial Sequence
  Homo sapiens siRNA sequence
tccaaagcgcaaagaagtt

SEQ ID NO: 227 - DNA, 19 bp, Artificial Sequence
  Homo sapiens siRNA sequence
ccaaagcgcaaagaagtta

SEQ ID NO: 228 - DNA, 19 bp, Artificial Sequence
  Homo sapiens siRNA sequence
gcaaagaagttatttgccg

SEQ ID NO: 229 - DNA, 19 bp, Artificial Sequence
  Homo sapiens siRNA sequence
gaagttatttgccgaggat

SEQ ID NO: 230 - DNA, 19 bp, Artificial Sequence
  Homo sapiens siRNA sequence
tcattctccttcaagggaa

SEQ ID NO: 231 - DNA, 19 bp, Artificial Sequence
  Homo sapiens siRNA sequence
gcttggagtttgtcatcct

SEQ ID NO: 232 - DNA, 19 bp, Artificial Sequence
  Homo sapiens siRNA sequence
tcatcctacaccaacctaa

SEQ ID NO: 233 - DNA, 25 bp, Artificial Sequence
  Homo sapiens siRNA sequence
tctacattccaaggagagatttaaa

SEQ ID NO: 234 - DNA, 25 bp, Artificial Sequence
  Homo sapiens siRNA sequence
caccatgaatcttccaaagcgcaaa

SEQ ID NO: 235 - DNA, 25 bp, Artificial Sequence
  Homo sapiens siRNA sequence
cgcaaagaagttatttgccgaggat

SEQ ID NO: 236 - DNA, 25 bp, Artificial Sequence
  Homo sapiens siRNA sequence
agaagttatttgccgaggatctgat

SEQ ID NO: 237 - DNA, 25 bp, Artificial Sequence
  Homo sapiens siRNA sequence
acaatatcattctccttcaagggaa

SEQ ID NO: 238 - DNA, 25 bp, Artificial Sequence
  Homo sapiens siRNA sequence
caatatcattctccttcaagggaat

SEQ ID NO: 239 - DNA, 25 bp, Artificial Sequence
  Homo sapiens siRNA sequence
caaatgtgttgttgaagctatttct

SEQ ID NO: 240 - DNA, 25 bp, Artificial Sequence
  Homo sapiens siRNA sequence
gcttggagtttgtcatcctacacca

SEQ ID NO: 241 - DNA, 25 bp, Artificial Sequence
  Homo sapiens siRNA sequence
agtttgtcatcctacaccaacctaa

SEQ ID NO: 242 - DNA, 25 bp, Artificial Sequence
  Homo sapiens siRNA sequence
catcctacaccaacctaattcaaat

(19) United States
(12) Patent Application Publication
(10) Pub. No.: US 2011/0038849 A1
(43) Pub. Date: Feb. 17, 2011

(54) INHIBITORY POLYNUCLEOTIDE COMPOSITIONS AND METHODS FOR TREATING CANCER

(75) Inventors: Frank Y. Xie, Germantown, MD (US); Patrick Y. Lu, Rockville, MD (US); Martin C. Woodle, Bethesda, MD (US); Yijia Liu, Gaithersburg, MD (US)

Correspondence Address: ROPES & GRAY LLP, PATENT DOCKETING 39/361, 1211 AVENUE OF THE AMERICAS, NEW YORK, NY, 10036-8704, US

(73) Assignee: Intradigm Corporation, Palo Alto, CA (US)

(21) Appl. No.: 12/519,944

(22) PCT Filed: Dec. 21, 2006
(86) PCT No.: PCT/US06/49261

§ 371 Date: Apr. 27, 2010

(51) Int. Cl. A61K 39/395; C07H 21/04; A61K 31/7088; C12N 5/02; C07K 16/00; A61P 35/00

(52) U.S. Cl. 424/130.1; 536/23.1; 514/44 R; 435/375; 530/387.1

(57) ABSTRACT

Compositions and methods for treating diseases, such as cancers. The compositions are effective to silence, down-regulate or suppress the expression of a validated target gene by stimulating the process of RNA interference of gene expression, thus inhibiting tumor growth. The invention also provides methods for treating diseases, such as cancers, by inactivation of a validated target gene product, using neutralizing antibody or small molecule drug, to inhibit tumor growth. More particularly, the compositions and methods are directed toward a cancer or a precancerous growth in a mammal, associated with pathological expression of a certain target genes identified herein. The compositions inhibit expression of the target gene when introduced into a tissue of the mammal. The methods include administering the compositions of the invention to a subject in need thereof in an amount effective to inhibit expression of a target gene in a cancerous tissue or organ.

### FIELD OF THE INVENTION

The present invention relates generally to polynucleotides useful to induce RNA interference as a modality in the treatment of cancer. More particularly, the invention relates to target oligonucleotide sequences directed toward certain genes implicated in the proliferation and/or metastasis of precancerous cells, cancer cells or tumor cells.

### BACKGROUND OF THE INVENTION

Cancer or pre-cancerous growth generally refers to malignant tumors, rather than benign tumors. Malignant tumors grow faster than benign tumors, and they penetrate and destroy local tissues. Some malignant tumors may spread by metastasis throughout the body via blood or the lymphatic system. The unpredictable and uncontrolled growth makes malignant cancers dangerous, and fatal in many cases.

Therapeutic treatment of malignant cancer is most effective at the early stage of cancer development. It is thus exceedingly important to identify and validate a therapeutic target in early tumor formation and to determine potent tumor growth or gene expression suppression elements or agents associated therewith.

RNA interference (RNAi) is a post-transcriptional process where in which double-stranded RNA (dsRNA) inhibits gene expression in a sequence specific fashion. The RNAi process occurs in at least two steps: in first step, the longer dsRNA is cleaved by an endogenous ribonuclease Dicer into shorter dsRNAs, termed “small interfering RNAs” or siRNAs that are typically less than 100-, 50-, 30-, 23-, or 21-nucleotides in length. In the second step, these siRNAs are incorporated into a multicomponent-ribonuclease called RNA-induced-silencing-complex (RISC; Hammond, S. M., et al., Nature (2000) 404:293-296). One strand of siRNA remains associated with RISC, and guides the complex towards a cognate RNA that has sequence complementary to the guider ss-siRNA in RISC. This siRNA-directed endonuclease digests the RNA, thereby inactivating it. This RNAi effect can be achieved by introducing either longer dsRNA or shorter siRNA to the target sequence within cells. It is also demonstrated that RNAi effect can be achieved by introducing plasmids that generate dsRNA complementary to target gene. See WO 99/32619 (Fire et al.); WO 99/53050 (Waterhouse et al.); WO 99/61631 (Heifetz et al.); Yang, D., et al., Curr. Biol. (2000)10:1191-1200), WO 00/44895 (Limmer); and DE 101 00 586.5 (Kreutzer et al.) for disclosures concerning RNAi in a wide range of organisms.

RNAi has been successfully used in gene function determination in Drosophila (Kennerdell et al. (2000) Nature Biotech 18: 896-898; Worby et al. (2001) Sci STKE Aug. 14, 2001(95):PL1; Schmid et al. (2002) Trends Neurosci 25(2):71-74; Hammond et al. (2000). Nature, 404: 293-298), C. elegans (Tabara et al (1998) Science 282: 430-431; Kamath et al. (2000) Genome Biology 2: 2.1-2.10; Grishok et al. (2000) Science 287: 2494-2497), and Zebrafish (Kennerdell et al. (2000) Nature Biotech 18: 896-898). There are numerous reports on RNAi effects in non-human mammalian and human cell cultures (Manche et al. (1992). Mol. Cell. Biol. 12:5238-5248; Minks et al. (1979). J. Biol. Chem. 254:10180-10183; Yang et al. (2001) Mol. Cell. Biol. 21(22):7807-7816; Paddison et al. (2002). Proc. Natl. Acad. Sci. USA 99(3):1443-1448; Elbashir et al. (2001) Genes Dev 15(2):188-200; Elbashir et al. (2001) Nature 411: 494-498; Caplen et al. (2001) Proc. Natl. Acad. Sci. USA 98: 9746-9747; Holen et al. (2002) Nucleic Acids Research 30(8):1757-1766; Elbashir et al. (2001) EMBO J 20: 6877-6888; Jarvis et al. (2001) TechNotes 8(5): 3-5; Brown et al. (2002) TechNotes 9(1): 3-5; Brummelkamp et al. (2002) Science 296:550-553; Lee et al. (2002) Nature Biotechnol. 20:500-505; Miyagishi et al. (2002) Nature Biotechnol. 20:497-500; Paddison et al. (2002) Genes & Dev. 16:948-958; Paul et al. (2002) Nature Biotechnol, 20:505-508; Sui et al. (2002) Proc. Natl. Acad. Sci. USA 99(6):5515-5520; Yu et al. (2002) Proc. Natl. Acad. Sci. USA 99(9):6047-6052).

### SUMMARY OF THE INVENTION

The present invention provides compositions and methods for treating diseases, such as cancers. The compositions are effective to silence, down-regulate or suppress the expression of a validated target gene by stimulating the process of RNA interference of gene expression. The compositions and methods thereby inhibit tumor growth. The invention also provides methods for treating diseases, such as cancers, by inactivation of a validated target gene product, using neutralizing antibody or small molecule drug, to inhibit tumor growth.

More particularly, the compositions and methods are directed toward a cancer or a precancerous growth in a mammal, associated with pathological expression of a target gene chosen from among an ICT-1053 gene, or an ICT-1052 gene, or an ICT-1027 gene, or an ICT-1051 gene, or an ICT-1054 gene, or an ICT-1020 gene, or an ICT-1021 gene, or an ICT-1022 gene (a “Target Gene” or “Target Genes” herein). The compositions inhibit expression of the target gene when introduced into a tissue of the mammal. The methods include administering the compositions of the invention to a subject in need thereof in an amount effective to inhibit expression of a target gene in a cancerous tissue or organ.

In a first aspect the invention provides an isolated targeting polynucleotide whose length is 200 or fewer nucleotides. This polynucleotide includes a first nucleotide sequence that targets a Target Gene or a complement thereto. The first nucleotide sequence or its complement is any number of nucleotides from 15 to 30 in length, and in several embodiments the length is 21 to 25 nucleotides.

In another aspect the polynucleotide of the invention described in the preceding paragraph further includes a second nucleotide sequence separated from the first nucleotide sequence by a loop sequence; the second nucleotide sequence
a) has substantially the same length as the first nucleotide sequence, and
b) is substantially complementary to the first nucleotide sequence, such that the polynucleotide forms a hairpin structure under conditions suitable for hybridization of the first and second nucleotide sequences.
   In many embodiments of the linear polynucleotide hairpin polynucleotide described in the preceding paragraphs, the first nucleotide sequence consists of
a) a sequence that targets a sequence chosen from SEQ ID NOS:7-76, 81-84, and 89-242 (a “Target Sequence” herein);
b) an extended sequence longer than, and containing, the targeting sequence given in item a), wherein the extended sequence targets a Target Gene, and the targeting sequence targets a Target Sequence;
c) a fragment of a sequence that targets a Target Sequence at least 15 nucleotides long, and shorter than the chosen Target Sequence;
d) a targeting sequence wherein up to 5 nucleotides differ from a chosen Target Sequence; or
e) a complement of a sequence given in a)-d).

In common embodiments the linear polynucleotide described herein consists of a Target Sequence, and optionally includes a dinucleotide overhang bound to the 3′ of the chosen sequence. In related common embodiments the hairpin polynucleotide described herein consists of a first chosen Target nucleotide Sequence, a loop sequence and the second nucleotide sequence substantially complementary to the Target Sequence.

In further embodiments the polynucleotide is a DNA, or an RNA, or the polynucleotide includes both deoxyribonucleotides and ribonucleotides.

In an additional aspect the invention provides a double stranded polynucleotide containing a first targeting linear polynucleotide strand described herein and a second polynucleotide strand including a second, nucleotide sequence that is substantially complementary to at least the first nucleotide sequence of the first polynucleotide strand and is hybridized thereto.

In still a further aspect the invention provides a combination or mixture of polynucleotides that includes a plurality of targeting linear polynucleotides, double stranded polynucleotides and/or hairpin polynucleotides described herein wherein each polynucleotide targets a different chosen Target Sequence in one or more chosen Target Genes.

In yet an additional aspect the invention provides a vector containing the targeting linear polynucleotide or the targeting hairpin polynucleotide described herein. In common embodiments the vector is a plasmid, a cosmid, a recombinant virus, a retroviral vector, an adenoviral vector, a transposon, or a minichromosome.

In further common embodiments of the vector a control element is operatively linked with the targeting polynucleotide effective to promote expression thereof. Additional aspects provide a cell transfected with one or more linear polynucleotides described herein or a cell transfected with one or more hairpin polynucleotides described herein, or a cell transfected with a combination of the said polynucleotides.

In still a further aspect the invention provides a pharmaceutical composition containing one or more linear polynucleotides or hairpin polynucleotides described herein, or a mixture thereof, wherein each polynucleotide targets a different Target Sequence in a Target Gene, or any two or more thereof, and a pharmaceutically acceptable carrier.

In yet an additional aspect the invention provides a method of synthesizing a polynucleotide having a sequence that targets a Target Gene described herein. The methods includes the steps of
a) providing a nucleotide reagent including a live reactive end and corresponding to the nucleotide at a first end of the sequence,
b) adding a further nucleotide reagent including a live reactive end and corresponding to a successive position of the sequence to react with the live reactive end from the preceding step and increase the length of the growing polynucleotide sequence by one nucleotide, and removing undesired products and excess reagent, and
c) repeating step b) until the nucleotide reagent corresponding to the nucleotide at a second end of the sequence has been added;
thereby providing the completed polynucleotide.

In still a further aspect the invention provides a method of inhibiting the growth of a cancer cell that includes contacting the cell with a composition containing one or more targeting linear polynucleotides or targeting hairpin polynucleotides described herein or a mixture thereof tinder conditions promoting incorporation of the one or more polynucleotides within the cell.

In an additional aspect the invention provides a method of promoting apoptosis in a cancer cell that includes contacting the cell with a composition containing one or more targeting linear polynucleotides or targeting hairpin polynucleotides described herein or a mixture thereof under conditions promoting incorporation of the one or more polynucleotides within the cell.

In yet another aspect, the invention provides methods for inhibiting cancer or precancerous growth in a mammalian tissue, wherein the method includes contacting the tissue with an inhibitory targeting polynucleotide of the invention that interacts with DNA or RNA that contains one or more Target Genes. The targeting polynucleotide inhibits expression of the one or more Target Genes in cells of the tissue. In several embodiments of this method the tissue is a breast tissue, colon tissue, a prostate tissue, a skin tissue, a bone tissue, a parotid gland tissue, a pancreatic tissue, a kidney tissue, a uterine cervix tissue, a lymph node tissue, or an ovarian tissue. Furthermore the inhibitory targeting polynucleotide is a nucleic acid molecule, a decoy molecule, a decoy DNA, a double stranded DNA, a single-stranded DNA, a complexed DNA, an encapsulated DNA, a viral DNA, a plasmid DNA, a naked RNA, an encapsulated RNA, a viral RNA, a double stranded RNA, a molecule, or combinations thereof.

In yet a further aspect the invention provides a use of a targeting linear polynucleotide or a targeting hairpin polynucleotide described herein, or of a mixture of two or more of them, wherein each polynucleotide targets a Target Gene, in the manufacture of a pharmaceutical composition effective to treat a cancer or a precancerous growth in a subject. In several embodiments of the use the cancer or the growth is found in a tissue chosen from breast tissue, colon tissue, prostate tissue, skin tissue, bone tissue, parotid gland tissue, pancreatic tissue, thyroid tissue, kidney tissue, uterine cervix tissue, lung tissue, lymph node tissue, hematopoietic tissue of bone marrow, or ovarian tissue. In additional common embodiments of the use the first nucleotide sequence in each polynucleotide consists of
a) a sequence that targets a sequence chosen from SEQ ID NOS:7-76, 81-84, and 89-242 (a “Target Sequence” herein);
b) an extended sequence longer than, and containing, the targeting sequence given in item a), wherein the extended sequence targets a Target Gene, and the targeting sequence targets a Target Sequence;
c) a fragment of a sequence that targets a Target Sequence at least 15 nucleotides long, and shorter than the chosen Target Sequence;
d) a targeting sequence wherein up to 5 nucleotides differ from a chosen Target Sequence; or
e) a complement of a sequence given in a)-d).
   In still further embodiments of the use the subject is a human.

In still a further aspect, the invention provides the use one or more antibodies directed against a product polypeptide of a Target Gene in the manufacture of a pharmaceutical composition effective to treat a cancer, a tumor or a precancerous growth in a subject.

### BRIEF DESCRIPTION OF THE DRAWINGS

FIG. 1. Schematic representation of various embodiments of the polynucleotides of the invention. Panel A, embodiments of a linear polynucleotide. The length is 200 nucleotides or less, and 15 nucleotides or greater. In b), a specified targeting sequence is contained within a larger targeting sequence. In d) the darker vertical bars diagrammatically represent substituted nucleotides. Panel B, an embodiment of a hairpin polynucleotide of overall length 200 nucleotides or less.

FIG. 2. Representation of the change in tumor size of MDA-MB-435 xenografts with time in response to transfection with ICT-1053 (PCDP10) siRNA, or with control siRNAs. Data are presented as Mean+/−SE.

FIG. 3. Representation of the change in tumor size of MDA-MB-435 xenografts with time in response to transfection with ICT-1052 (cMet) siRNA, or with control siRNAs. Data are presented as Mean+/−SE.

FIG. 4. Representation of the change in tumor size of A549 xenografts with time in response to transfection with ICT-1052 (cMet) siRNA, or with a control siRNA. Data are presented as Mean+/−SE.

FIG. 5. Representation of the inhibition of proliferation of MDA-MB-435 cells in culture when treated with ICT-1052 siRNA or ICT-1053 siRNA, or a control siRNA. Data are presented as mean values.

FIG. 6. Representation of the inhibition of proliferation of HCT116 human colon carcinoma cells in culture when treated with ICT-1052 siRNA or ICT-1053 siRNA, or a control siRNA. Data are presented as mean+/−SE.

FIG. 7. Representation of the inhibition of proliferation of A549 human lung carcinoma cells in culture when treated with ICT-1052 siRNA or a control siRNA. Data are presented as mean+/−SE.

FIG. 8. Representation of the change in tumor size of MDA-MB-435 xenografts with time in response to transfection with ICT-1027 (GRB2 BP) siRNA or a control siRNA. Data were presented as mean+/−SE.

FIG. 9. Representation of the induction of apoptosis in MDA-MB-435 cells in response to treatment with ICT-1027 siRNA, or control siRNA. Data are presented as mean+/−SE.

FIG. 10. Representation of the change in tumor size of MDA-MB-435 xenografts with time in response to transfection with ICT-1051 (A-Raf) siRNA or a control siRNA. Data were presented as mean+/−SE.

FIG. 11. Representation of the change in tumor size of MDA-MB-435 xenografts with time in response to transfection with ICT-1054 (PCDP6) siRNA or a control siRNA. Data were presented as mean+/−SE.

FIG. 12. Representation of the change in tumor size of MDA-MB-435 xenografts with time in response to transfection with ICT-1020 (Dicer) siRNA or a control siRNA. Data were presented as mean+/−SE.

FIG. 13. Representation of the change in tumor size of MDA-MB-435 xenografts with time in response to transfection with ICT-1021 (MD2 protein) siRNA or a control siRNA. Data were presented as mean+/−SE.

FIG. 14. Representation of the change in tumor size of MDA-MB-435 xenografts with time in response to transfection with ICT-1022 (GAGE-2) siRNA or a control siRNA. Data were presented as mean+/−SE.

### DETAILED DESCRIPTION OF THE INVENTION

All patents, patent application publications, and patent applications identified herein are incorporated by reference in their entireties, as if appearing herein verbatim. All technical publications identified herein are also incorporated by reference.

In the present description, the articles “a”, “an”, and “the” relate equivalently to a meaning as singular or as plural. The particular sense for these articles is apparent from the context in which they are used.

As used herein the term “tumor” refers to all neoplastic cell growth and proliferation, whether malignant or benign, and all precancerous and cancerous cells and tissues.

As used herein the term “precancerous” refers to cells or tissues having characteristics relating to changes that may lead to malignancy or cancer.

As used herein the term “cancer” refers to cells or tissues possessing characteristics such as uncontrolled proliferation, loss of specialized functions, immortality, significant metastatic potential, significant increase in anti-apoptotic activity, rapid growth and proliferation rate, and certain characteristic morphological and cellular markers. In some circumstances, cancer cells will be in the form of a tumor; such cells may exist locally within an animal, and in other circumstances they may circulate in the blood stream as independent cells, for example, leukemic cells.

As used herein the term “target” sequence and similar terms and phrases relate to a nucleotide sequence that occurs in a nucleic acid of a cancer cell against which a polynucleotide of the invention is directed. A “target gene” refers to an expressed gene wherein modulation of the level of gene expression or of gene product activity prevents and/or ameliorates disease progression. In particular, target genes in the present invention include endogenous genes and their variants, as described herein.

A targeting polynucleotide targets a cancer cell nucleic acid sequence either a) by including a sequence whose complement is homologous or identical to a particular subsequence (termed a target sequence) contained within the genome of the pathogen, or b) by including a sequence that is itself homologous or identical to the target sequence. A targeting polynucleotide that is effective within a cell is a double stranded molecule comprised of one of each the strands specified in a) and b). It is believed that any double stranded targeting polynucleotide so targeting a cancer cell nucleic acid sequence has the ability to hybridize with the target sequence according to the RNA interference phenomenon, thereby initiating RNA interference.

A target gene in a subject may have a sequence that is identical to a wild type sequence identified, for example, in various GenBank accession entries, and in entries in similar databases; typically such databases are accessible to the public. An interfering RNA to be used to suppress expression of a target gene may, however, not, be perfectly complementary to its target, or the target may differ from a sequence considered to be a wild type sequence given by an existing GenBank accession number. For example, a target gene may include one or more single polynucleotide polymorphisms, and thus differ slightly from the sequence in a GenBank accession number. In addition a target gene may produce an mRNA that is the product of alternative splicing of exons, resulting in a mature mRNA that has fewer exons than the chromosomal gene. Such an alternatively spliced mRNA can also be a target of an RNAi species directed against the wild type gene. In the present disclosure all such eventualities are encompassed within the notion of a target gene, and any RNAi species developed to target the wild type sequence potentially targets such altered or modified transcripts and is included within the notion of a targeting sequence.

In general, a “gene” is a region in the genome that is capable of being transcribed to an RNA that either has a regulatory function, a catalytic function, and/or encodes a protein. A eukaryotic gene typically has introns and exons, which may organize to produce different RNA splice variants that encode alternative versions of a mature protein. The skilled artisan will appreciate that the present invention encompasses all endogenous genes that may be found, including splice variants, allelic variants and transcripts that occur because of alternative promoter sites or alternative polyadenylation sites. The endogenous gene, as described herein, also can be a mutated endogenous gene, wherein the mutation can be in the coding or regulatory regions.

“Antisense RNA”: In eukaryotes, RNA polymerase catalyzes the transcription of a structural gene to produce mRNA. A DNA molecule can be designed to contain an RNA polymerase template in which the RNA transcript has a sequence that is complementary to that of a preferred mRNA. The RNA transcript is termed an “antisense RNA.” Antisense RNA molecules can inhibit mRNA expression (for example, Rylova et al., Cancer Res, 62(3):801-8, 2002; Shim et al., Int. J. Cancer, 94(1):6-15, 2001).

“Antisense DNA” or “DNA decoy” or “decoy molecule”: With respect to a first nucleic acid molecule, a second DNA molecule or a second chimeric nucleic acid molecule that is created with a sequence, which is a complementary sequence or homologous to the complementary sequence of the first molecule or portions thereof, is referred to as the antisense DNA or DNA decoy or decoy molecule of the first molecule. The term “decoy molecule” also includes a nucleic molecule, which may be single or double stranded, that comprises DNA or PNA (peptide nucleic acid) (Mischiati et al., Int. J. Mol. Med., 9(6):633-9, 2002), and that contains a sequence of a protein binding site, preferably a binding site for a regulatory protein and more preferably a binding site for a transcription factor. Applications of antisense nucleic acid molecules, including antisense DNA and decoy DNA molecules are known in the art, for example, Morishita et al., Ann. N Y Acad. Sci., 947:294-301, 2001; Andratschke et al., Anticancer Res, 21:(5)3541-3550, 2001.

“Stabilized RNA”: A stabilized RNAi, siRNA or a shRNA as described herein, is protected against degradation by exonucleases, including RNase, for example, using a nucleotide analogue that is modified at the 3′ position of the ribose sugar (for example, by including a substituted or unsubstituted alkyl, alkoxy, alkenyl, alkenyloxy, alkynyl or alkynyloxy group as defined above), or modified elsewhere in its structure to achieve protection. The RNAi, siRNA or a shRNA also can be stabilized against degradation at the 3′ end by exonucleases by including a 3′-3′-linked dinucleotide structure (Ortigao et al., Antisense Research and Development 2:129-146 (1992)) and/or two modified phospho bonds, such as two phosphorothioate bonds.

“Encapsulated nucleic acids”, including encapsulated DNA or encapsulated RNA, refer to nucleic acid molecules in microsphere or microparticle and coated with materials that are relatively non-immunogenic and subject to selective enzymatic degradation, for example, synthesized microspheres or microparticles by the complex coacervation of materials, for example, gelatin and chondroitin sulfate (see, for example, U.S. Pat. No. 6,410,517). Encapsulated nucleic acids in a microsphere or a microparticle are encapsulated in such a way that it retains its ability to induce expression of its coding sequence (see, for example, U.S. Pat. No. 6,406,719).

“Inhibitors” refers to molecules that inhibit and/or block an identified function. Any molecule having potential to inhibit and/or block an identified function can be a “test molecule,” as described herein. For example, referring to oncogenic function or anti-apoptotic activity of a Target Gene, such molecules may be identified using in vitro and in vivo assays of the particular Target Gene. Inhibitors are compounds that partially or totally block Target Gene activity, decrease, prevent, or delay their activation, or desensitize its cellular response. This may be accomplished by binding to Target Gene products, i.e. proteins, directly or via other intermediate molecules. An antagonist or an antibody, e.g. monoclonal or polyclonal antibody, that blocks gene product activity of a Target Gene, including inhibition of oncogenic function or anti-apoptotic activity of a Target Gene, is considered to be such an inhibitor. Inhibitors according to the instant invention is: a siRNA, an RNAi, a shRNA, an antisense RNA, an antisense DNA, a decoy molecule, a decoy DNA, a double stranded DNA, a single-stranded DNA, a complexed DNA, an encapsulated DNA, a viral DNA, a plasmid DNA, a naked RNA, an encapsulated RNA, a viral RNA, a double stranded RNA, a molecule capable of generating RNA interference, or combinations thereof. The group of inhibitors of this invention also includes genetically modified versions of Target Genes, for example, versions with altered activity. The group thus is inclusive of the naturally occurring protein as well as synthetic ligands, antagonists, agonists, antibodies, small chemical molecules and the like.

“Assays for inhibitors” refer to experimental procedures including, for example, expressing Target Genes in vitro, in cells, applying putative inhibitor compounds, and then determining the functional effects on Target Gene activity or transcription. Samples that contain or are suspected of containing a Target Gene are treated with a potential inhibitor. These inhibitors include nucleic acid based molecules, such as siRNA, antisense, double-stranded RNA and DNA, or double-stranded RNA/DNA, ribozyme and triplex, etc.; and protein based molecules, such as peptides, synthetic ligands, truncated partial proteins, soluble receptors, monoclonal antibody, polyclonal antibody, intrabody and single chain antibody, etc.; as well as small chemical molecules at various forms. The extent of inhibition or change is examined by comparing the activity measurement from the samples of interest to control samples. A threshold level is established to assess inhibition. For example, inhibition of a Target Gene product polypeptide is considered achieved when the Target Gene activity value relative to a suitable control is 80% or lower.

As used herein, a first sequence or subsequence is “identical”, or has “100% identity”, or is described by a term or phrase conveying the notion of 100% identity, to a second sequence or subsequence when the first sequence or subsequence has the same base as the second sequence or subsequence at every position of the sequence or subsequence. In determining identity, any T (thymidine) or any derivative thereof, or a U (uridine) or any derivative thereof, are equivalent to each other, and thus identical. No gaps are permitted for a first and second sequence to be identical.

A sequence of a targeting polynucleotide, or its complement, may be completely identical to the target sequence, or it may include mismatched bases at particular positions in the sequence. Incorporation of mismatches is described fully herein. Without wishing to be bound by theory, it is believed that incorporation of mismatches provides an intended degree of stability of hybridization under physiological conditions to optimize the RNA interference phenomenon for the particular target sequence in question. The extent of identity determines the percent of the positions in the two sequences whose bases are identical to each other. The “percentage of sequence identity” is calculated as shown

$$\% Identity = \frac{Number of identical bases}{Total number of bases} \times 100$$

Sequences that are less than 100% identical to each other are “similar” or “homologous” to each other; the degree of homology or the percent similarity are synonymous terms relating to the percent of identity between two sequences or subsequences. For example, two sequences displaying at least 60% identity, or preferably at least 65% identity, or preferably at least 70% identity, or preferably at least 75% identity, or preferably at least 80% identity, or more preferably at least 85% identity, or more preferably at least 90% identity, or still more preferably at least 95% identity, to each other are “similar” or “homologous” to each other. Alternatively; with reference to the oligonucleotide sequence of an siRNA molecule, two sequences that differ by 5 or fewer bases, or by 4 or fewer bases, or by 3 or fewer bases, or by two or fewer bases, or by one base, are termed “similar” or “homologous” to each other.

“Identity” and “similarity” can additionally be readily calculated by known methods, including but not limited to those described in Computational Molecular Biology, Lesk. A. M., ed., Oxford University Press, New York, 1988; Biocomputing: Informatics and Genome Projects, Smith, D. W., ed., Academic Press, New York, 1993; Computer Analysis of Sequence Data, Part I. Griffin, A. M., and Griffin, H. G., eds. Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press. New York, 1991; and Carillo, H., and Lipman, D., SIAM J. Applied Math. (1988) 48: 1073. Methods of alignment of sequences for comparison are well-known in the art. Optimal alignment of sequences for comparison may be conducted by the local homology algorithm of Smith and Waterman, Adv. Appl. Math., 2: 482, 1981; by the homology alignment algorithm of Needleman and Wunsch, J. Mol. Biol., 48: 443, 1970; by the search for similarity method of Pearson and Lipman, Proc. Natl. Acad. Sci. USA, 8: 2444, 1988; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp, Gene, 73: 237-244, 1988; Corpet, et al., Nucleic Acids Research, 16:881-90, 1988; Huang, et al., Computer Applications in the Biosciences, 8:1-6, 1992; and Pearson, et al., Methods in Molecular Biology, 24:7-331, 1994. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995.

Unless otherwise stated, sequence identity/similarity values provided herein refer to the value obtained using, the BLAST 2.0 suite of programs, or their successors, using default parameters. Altschul et al., Nucleic Acids Res, 2:3389-3402, 1997. It is to be understood that default settings of these parameters can be readily changed as needed in the future.

The term “substantial identity” or “homologous” in their various grammatical forms means that a polynucleotide comprises a sequence that has a desired identity, for example, at least 60% identity, preferably at least 70% sequence identity, more preferably at least 80%, still more preferably at least 90% and even more preferably at least 95%, compared to a reference sequence using one of the alignment programs described.

As used herein, the term “isolated”, and similar words, when used to describe a nucleic acid, a polynucleotide, or an oligonucleotide relate to the composition being removed from its natural or original state. Thus, if it occurs in nature, it has been removed from its original environment. If it has been prepared synthetically, it has been removed from an original product mixture resulting from the synthesis. For example, a naturally occurring polynucleotide naturally present in a living organism in its natural state is not “isolated,” but the same polynucleotide separated from at least one material with which it coexists in its natural state is “isolated”, as the term is employed herein. Generally, removal of at least one coexisting material constitutes “isolating” a nucleic acid, a polynucleotide, an oligonucleotide. In many cases several, many, or most coexisting materials may be removed to isolate the nucleic acid, polynucleotide, or oligonucleotide. A nucleic acid, a polynucleotide, or an oligonucleotide that is the product of an in vitro synthetic process or a chemical synthetic process is essentially isolated as the result of the synthetic process. In important embodiments such synthetic products are treated to remove reagents and precursors used, and side products produced, by the process.

Polynucleotides incorporated into a composition, such as a formulation, a transfecting composition, a pharmaceutical composition, or compositions or solutions for chemical or enzymatic reactions, which are not naturally occurring compositions, remain isolated polynucleotides or polypeptides within the meaning of that term as it is employed herein.

As used herein, a “nucleic acid” or “polynucleotide”, and similar terms and phrases, relate to polymers composed of naturally occurring nucleotides as well as to polymers composed of synthetic or modified nucleotides. Thus, as used herein, a polynucleotide that is a RNA, or a polynucleotide that is a DNA, or a polynucleotide that contains both deoxyribonucleotides and ribonucleotides, may include naturally occurring moieties such as the naturally occurring bases and ribose or deoxyribose rings, or they may be composed of synthetic or modified moieties such as those described below. A polynucleotide employed in the invention may be single stranded or it may be a base paired double stranded structure, or even a triple stranded base paired structure.

Nucleic acids and polynucleotides may be 20 or more nucleotides in length, or 30 or more nucleotides in length, or 50 or more nucleotides in length, or 100 or more, or 1000 or more, or tens of thousands or more, or, hundreds of thousands or more, in length. An siRNA may be a polynucleotide as defined herein. As used herein, “oligonucleotides” and similar terms based on this relate to short polymers composed of naturally occurring nucleotides as well as to polymers composed of synthetic or modified nucleotides, as described in the immediately preceding paragraph. Oligonucleotides may be 10 or more nucleotides in length, or 15, or 16, or 17, or 18, or 19, or 20 or more nucleotides in length, or 21, or 22, or 23, or 24 or more nucleotides in length, or 25, or 26, or 27, or 28 or 29, or 30 or more nucleotides in length, 35 or more, 40 or more, 45 or more, up to about 50, nucleotides in length. An oligonucleotide sequence employed as a targeting sequence in an siRNA may have any number of nucleotides between 15 and 30 nucleotides. In many embodiments an siRNA may have any number of nucleotides between 21 and 25 nucleotides. Oligonucleotides may be chemically synthesized and may be used as siRNAs, PCR primers, or probes.

It is understood that, because of the overlap in size ranges provided in the preceding paragraph, the terms “polynucleotide” and “oligonucleotide” may be used synonymously herein to refer to an siRNA of the invention.

As used herein “nucleotide sequence”, “oligonucleotide sequence” or “polynucleotide sequence”, and similar terms, relate interchangeably both to the sequence of bases that an oligonucleotide or polynucleotide has, as well as to the oligonucleotide or polynucleotide structure possessing the sequence. A nucleotide sequence or a polynucleotide sequence furthermore relates to any natural or synthetic polynucleotide or oligonucleotide in which the sequence of bases is defined by description or recitation of a particular sequence of letters designating bases as conventionally employed in the field.

A “nucleoside” is conventionally understood by workers of skill in fields such as biochemistry, molecular biology, genomics, and similar fields related to the field of the invention as comprising a monosaccharide linked in glycosidic linkage to a purine or pyrimidine base; and a “nucleotide” comprises a nucleoside with at least one phosphate group appended, typically at a 3′ or a 5′ position (for pentoses) of the saccharide, but may be at other positions of the saccharide. Nucleotide residues occupy sequential positions in an oligonucleotide or a polynucleotide. A modification or derivative of a nucleotide may occur at any sequential position in an oligonucleotide or a polynucleotide. All modified or derivatized oligonucleotides and polynucleotides are encompassed within the invention and fall within the scope of the claims. Modifications or derivatives can occur in the phosphate group, the monosaccharide or the base.

By way of nonlimiting examples, the following descriptions provide certain modified or derivatized nucleotides, all of which are within the scope of the polynucleotides of the invention. The monosaccharide may be modified by being, for example, a pentose or a hexose other than a ribose or a deoxyribose. The monosaccharide may also be modified by substituting hydroxyl groups with hydro or amino groups, by alkylating or esterifying additional hydroxyl groups, and so on. Substituents at the 2′ position, such as 2′-O-methyl, 2′-O-ethyl, 2′-O-propyl, 2′-O-allyl, 2′-O-aminoalkyl or 2′-deoxy-2′-fluoro group provide enhanced hybridization properties to an oligonucleotide.

The bases in oligonucleotides and polynucleotides may be “unmodified” or “natural” bases include the purine bases adenine (A) and guanine (G), and the pyrimidine bases thymine (T), cytosine (C) and uracil (U). In addition they may be bases with modifications or substitutions. Nonlimiting examples of modified bases include other synthetic and natural bases such as hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil, 5-halo-cytosine, 5-propy-uracil, 5-propynyl-cytosine and other alkynyl derivatives of pyrimidine bases, 6-azo-uracil, 6-azo-cytosine, 6-azo-thymine, 5-uracil (pseudouracil), 4-thiouracil, 8-halo, 8-amino-, 8-thiol-, 8-thioalkyl-, 8-hydroxyl- and other 8-substituted adenines and guanines, 5-halo particularly 5-bromo, 5-trifluoromethyl and other 5-substituted uracils and cytosines, 7-methylguanine and 7-methyladenine, 2-fluoro-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′, 2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition (1991) 30, 613, and those disclosed by Sanghvi, Y. S., Chapter 15, Antisense Research and Applications, pages 289-302, Crooke, S. T. and Lebleu, B., ed., CRC Press, 1993. Certain of these bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications. See U.S. Pat. Nos. 6,503,754 and 6,506,735 and references cited therein, incorporated herein by reference. Modifications further include those disclosed in U.S. Pat. Nos. 5,138,045 and 5,218,105, drawn to polyamine conjugated oligonucleotides; U.S. Pat. Nos. 5,212,295, 5,521,302, 5,587,361 and 5,599,797, drawn to oligonucleotides incorporating chiral phosphorus linkages including phosphorothioates; U.S. Pat. Nos. 5,378,825, 5,541,307, and 5,386,023, drawn to oligonucleotides having modified backbones; U.S. Pat. Nos. 5,457,191 and 5,459,255, drawn to modified nucleobases; U.S. Pat. No. 5,539,082, drawn to peptide nucleic acids; U.S. Pat. No. 5,554,746, drawn to oligonucleotides having beta-lactam backbones; U.S. Pat. No. 5,571,902, disclosing the synthesis of oligonucleotides; U.S. Pat. No. 5,578,718, disclosing alkylthio nucleosides; U.S. Pat. No. 5,506,351, drawn to 2′-O-alkyl guanosine, 2,6-diaminopurine, and related compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,223,168, and U.S. Pat. No. 5,608,046, drawn to conjugated 4′-desmethyl nucleoside analogs; U.S. Pat. Nos. 5,602,240, and 5,610,289, drawn to backbone-modified oligonucleotide analogs; U.S. Pat. Nos. 6,262,241, and 5,459,255, drawn to, inter alia, methods of synthesizing 2′-fluoro-oligonucleotides.

The linkages between nucleotides is commonly the 3′-5′ phosphate linkage, which may be a natural phosphodiester linkage, a phosphothioester linkage, and still other synthetic linkages. Oligonucleotides containing phosphorothioate backbones have enhanced nuclease stability. Examples of modified backbones include, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates. Additional linkages include phosphotriester, siloxane, carbonate, carboxymethylester, acetamidate, carbamate, thioether, bridged phosphoramidate, bridged methylene phosphonate, bridged phosphorothioate and sulfone internucleotide linkages. Other polymeric linkages include 2′-5′ linked analogs of these. See U.S. Pat. Nos. 6,503,754 and 6,506,735 and references cited therein, incorporated herein by reference.

Any modifications including those exemplified in the above description can readily be incorporated into, and are comprised within the scope of, the targeting polynucleotides of the invention. Use of any modified nucleotide is equivalent to use of a naturally occurring nucleotide having the same base-pairing properties, as understood by a worker of skill in the art. All equivalent modified nucleotides fall within the scope of the present invention as disclosed and claimed herein.

As used herein and in the claims, the term “complement”, “complementary”, “complementarity”, and similar words and phrases, relate to two sequences whose bases form complementary base pairs, base by base, as conventionally understood by workers of skill in fields such as biochemistry, molecular biology, genomics, and similar fields related to the field of the invention. Two single stranded (ss) polynucleotides having complementary sequences can hybridize with each other under suitable buffer and temperature conditions to form a double stranded (ds) polynucleotide. By way of nonlimiting example, if the naturally occurring bases are considered, A and (T or U) interact with each other, and G and C interact with each other. Unless otherwise indicated, “complementary” is intended to signify “fully complementary”, namely, that when two polynucleotide strands are aligned with each other, there will be at least a portion of the strands in which each base in a sequence of contiguous bases in one strand is complementary to an interacting base in a sequence of contiguous bases of the same length on the opposing strand.

As used herein, “hybridize”, “hybridization” and similar words and phrases relate to a process of forming a nucleic acid, polynucleotide, or oligonucleotide duplex by causing strands with complementary sequences to interact with each other. The interaction occurs by virtue of complementary bases on each of the strands specifically interacting to form a pair. The ability of strands to hybridize to each other depends on a variety of conditions, as set forth below. Nucleic acid strands hybridize with each other when a sufficient number of corresponding positions in each strand are occupied by nucleotides that can interact with each other. Polynucleotide strands that hybridize to each other may be fully complementary. Alternatively, two hybridized polynucleotides may be “substantially complementary” to each other, indicating that they have a small number of mismatched bases. Both naturally occurring bases, and modified bases such as those described herein, participate in forming complementary base pairs. It is understood by workers of skill in the field of the present invention, including by way of nonlimiting example biochemists and molecular biologists, that the sequences of strands forming a duplex need not be 100% complementary to each other to be specifically hybridizable.

As used herein, a “nucleotide overhang” and similar terms and phrases relate to an unpaired nucleotide, or nucleotides that extend beyond the duplex structure of a double stranded polynucleotide when a 3′-end of one strand of the duplex extends beyond the 5′-end of the other strand, or mutatis mutandi. Conversely “blunt” or “blunt end” and similar terms and phrases relate to a duplex having no unpaired nucleotides at an end of the duplex, i.e., no nucleotide overhang.

As used herein, “antisense strand” and similar terms and phrases relate to a strand of a polynucleotide duplex which includes a region that is substantially complementary to a target sequence. As used herein, the term “region of complementarity” refers to the region on the antisense strand that is substantially complementary to a sequence, for example a target sequence, as defined herein. If a region of complementarity is not fully complementary to a target sequence, mismatches are commonly tolerated in the terminal regions and, if present, are commonly within 6, 5, 4, 3, or 2 nucleotides of the 5′ and/or 3′ terminus.

The term “sense strand” and similar terms and phrases as used herein, relate to a strand of a polynucleotide duplex that includes a region that is complementary to a region of the antisense strand of a target sequence. Thus a sense strand has a region that is identical or substantially similar to the target sequence.

As used herein “fragment” and similar words and phrases relate to portions of a nucleic acid, polynucleotide or oligonucleotide shorter than the full sequence of a reference. The sequence of bases in a fragment is unaltered from the sequence of the corresponding portion of the reference; there are no insertions or deletions in a fragment in comparison with the corresponding portion of the reference. As contemplated herein, a fragment of a nucleic acid or polynucleotide, such as an oligonucleotide, is 15 or more bases in length, or 16 or more, 17 or more, 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 50 or more, or 75 or more, or 100 or more bases in length, up to a length that is one base shorter than the full length sequence.

As used herein the terms “pathological expression” and “pathogenic expression”, and similar phrases, will together be referred to as “pathological expression”, and relate to differential expression of a gene which is associated with a pathogenic state or a pathological condition. Pathological expression thus relates to expression of a gene that differs from the expression level found in a non-diseased condition, or a non-pathological condition. In the present disclosure, pathological expression relates especially to gene identified as a target gene, i.e., a gene that is a target for RNAi therapy. Thus, although pathological expression may generally relate to both overexpression of a gene and underexpression of a gene, the pathological expression of a gene to be targeted by RNAi therapy is generally overexpression, and the RNAi therapy is intended to inhibit or reduce the overexpression.

A full-length gene or RNA further encompasses any naturally occurring splice variants, allelic variants, other alternative transcripts, splice variants that exhibit the same or a similar function as the naturally occurring full length gene, and the resulting RNA molecules. A fragment of a gene can be any portion from the gene, which may or may not represent a functional domain, for example, a catalytic domain, a DNA binding domain, etc.

“Complementary DNA” (cDNA), is a single-stranded DNA molecule that is copied from an mRNA template by the enzyme reverse transcriptase, resulting in a sequence complementary to that of the mRNA. Those skilled in the an also use the term “cDNA” to refer to a double-stranded DNA molecule that comprises such a single-stranded DNA molecule and its complementary DNA strand.

The term “operably linked” and similar terms and phrases are used to describe the connection between regulatory elements and a gene or its coding region. That is, gene expression is typically governed by certain transcriptional regulatory elements, including constitutive or inducible promoters, tissue-specific regulatory elements, and enhancers. Such a gene or coding region is then said to be “operably linked to” or “operatively linked to” or “operably associated with” the regulatory elements, meaning that the gene or coding region is controlled or influenced by the regulatory element.

As used herein the terms “interfere”, “silence” and “inhibit the expression of”, and similar terms and phrases, in as far as they refer to a target gene, relate to suppression or inhibition of expression of a target either partially or essentially completely. Frequently such interference is manifested as a suppressed phenotype. In various cases expression of the target gene is suppressed by at least about 10%, or about 20%, or about 30%, or about 40%, or about 50%, or about 60%, or about 70%, or about 80% by, administration of a targeting polynucleotide of the invention. In favorable embodiments, the target gene is suppressed by at least about 85%, or about 90%, or about 95%, or substantially completely, by administering a targeting polynucleotide. Such interference may be manifested in cells in a cell culture, or in a tissue explant, or in vivo in a subject.

As used herein, the term “treatment” and similar terms and phrases relate to the application or administration of a therapeutic agent to a subject having a disease or condition, a symptom of disease, or a predisposition toward a disease, or application or administration of a therapeutic agent to an isolated tissue or cell line from the subject. Treatment is intended to promote curing or healing thereof, or to alleviate, relieve, alter, remedy, ameliorate, improve, or affect the disease, the symptoms of disease, or the predisposition toward disease.

As used herein, the phrases “therapeutically effective amount” and “prophylactically effective amount” refer to an amount that provides a therapeutic benefit in the treatment of a disease, or an effect providing prevention or diminishing the severity of the disease, respectively. The specific amount that is therapeutically effective can be readily determined by an ordinary medical practitioner employing assessment of response in a treated subject, and may vary depending on factors known in the art, such as the nature of the disease, the subject's history and age, the stage of disease, and the administration of other therapeutic agents.

As used herein, a “pharmaceutical composition” relates to a composition that includes a pharmacologically effective amount of a targeting polynucleotide and a pharmaceutically acceptable carrier. As used herein, “pharmacologically effective amount,” “therapeutically effective amount” or simply “effective amount” refers to that amount of an inhibitory polynucleotide effective to produce the intended pharmacological, therapeutic or preventive result. For example, if a given clinical treatment is considered effective when there is at least a minimal measurable change in a clinical parameter associated with a disease or disorder, a therapeutically effective amount of a drug for the treatment of that disease or disorder is the amount necessary to effect at least extent of change in the parameter.

The term “pharmaceutically acceptable carrier” refers to a composition for administration of a therapeutic agent that is at least both physiologically acceptable and approvable by a regulatory agency.

Nucleotides may also be modified to harbor a label. Nucleotides bearing a fluorescent label or a biotin label, for example, are available from Sigma (St. Louis, Mo.).

RNA Interference

According to the invention, gene expression of targets in cancer cells that promote proliferation and/or metastasis is attenuated by RNA interference. In particular, genes targeted in the present invention include those designated ICT-1052, ICT-1053, ICT-1027, ICT-1051, ICT-1054, ICT-1020, ICT-1021 and ICT-1022. Transcription products of a Target Gene are targeted within a cell by specific double stranded siRNA nucleotide sequences that are complementary to at least a segment of the target that contains any number of nucleotides between 15 and 30, or in many cases, contains anywhere between 21 and 25 nucleotides. The target may occur in the 5′ untranslated (UT) region, in a coding sequence, or in the 3′ UT region. See, e.g., PCT applications WO00/44895, WO99/32619, WO01/75164, WO01/92513, WO01/29058, WO01/89304, WO02/16620, and WO02/29858, each incorporated by reference herein in their entirety.

According to the methods of the present invention, cancer cell gene expression, and thereby cancer cell replication, is suppressed using siRNA. A targeting polynucleotide according to the invention includes an siRNA oligonucleotide. An siRNA can be prepared by chemical synthesis of nucleotide sequences identical or similar to a cancer cell target sequence. See, e.g., Tuschl, Zamore, Lehmann, Bartel and Sharp (1999), Genes & Dev. 13: 3191-3197, incorporated herein by reference in its entirety. Alternatively, a targeting siRNA can be obtained using a targeting polynucleotide sequence, for example, by digesting a cancer cell ribopolynucleotide sequence in a cell-free system, such as but not limited to a Drosophila extract, or by transcription of recombinant double stranded cancer cell cRNA.

Efficient silencing is generally observed with siRNA duplexes composed of a 15-30 nt strand complementary (i.e. antisense) to the chosen target sequence and a 15-30 nt sense strand of the same length. In many embodiments each strand of an siRNA paired duplex has in addition an overhang of 1, 2, 3, or 4 unpaired nucleotides at the 3′ end. In common embodiments the size of the overhang is 2 nt. The sequence of the 3′ overhang makes an additional small contribution to the specificity of siRNA target recognition. In one embodiment, the nucleotides in the 3′ overhang are ribonucleotides. In an alternative embodiment, the nucleotides in the 3′ overhang are deoxyribonucleotides. Use of 3′ deoxynucleotides in a 3′ overhang provides enhanced intracellular stability.

A recombinant expression vector of the invention that includes a targeting sequence, when introduced within a cell, is processed to provide an RNA that includes an siRNA sequence targeting a gene in a cancer cell implicated in cell proliferation and/or metastasis. Such a vector is a DNA molecule cloned into an expression vector comprising operatively-linked regulatory sequences flanking the cancer cell targeting sequence in a manner that allows for expression of the targeting sequence. From the vector, an RNA molecule that is antisense to cancer cell RNA is transcribed by a first promoter (e.g., a promoter sequence 3′ of the cloned DNA) and an RNA molecule that is the sense strand for the cancer cell RNA target is transcribed by a second promoter (e.g., a promoter sequence 5′ of the cloned DNA). The sense and antisense strands then hybridize in vivo to generate siRNA constructs targeting the cancer cell RNA molecule for silencing of the gene. Alternatively, two separate constructs can be utilized to create the sense and anti-sense strands of a siRNA construct. Further, cloned DNA can encode a transcript having a hairpin secondary structure, wherein a single transcript has both the sense and complementary antisense sequences from the target gene or genes. In an example of this embodiment, a hairpin RNAi transcription product includes a first sequence that is similar to all or a portion of the target gene and a second sequence complementary to the first sequence, so disposed as to form a hairpin duplex. In another example, a hairpin RNAi product is a siRNA. The regulatory sequences flanking the cancer cell sequence in the vector may be identical or may be different, such that their expression may be modulated independently, or in a temporal or spatial manner.

In certain embodiments, siRNAs are transcribed intracellularly by cloning the cancer cell Target Gene templates into a vector containing, e.g., a RNA poi III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoters, therefore they are ideally suited for the expression of around 21-nucleotide siRNAs in, e.g., an approximately 50-nucleotide RNA hairpin-loop transcript. An initial BLAST homology search for the selected siRNA sequence is done against an available nucleotide sequence library to ensure that only an intended target preferentially expressed in a cancer cell, but no nontargeted host gene, is identified. See, Elbashir et al. 2001 EMBO J. 20(23):6877-88.

Synthesis of Polynucleotides

Oligonucleotides corresponding to targeting nucleotide sequences, and polynucleotides that include targeting sequences, can be prepared by standard synthetic techniques, e.g., using an automated DNA synthesizer. Methods for synthesizing oligonucleotides include well-known chemical processes, including, but not limited to, sequential addition of nucleotide phosphoramidites onto surface-derivatized particles, as described by T. Brown and Dorcas J. S. Brown in Oligonucleotides and Analogues A Practical Approach, F. Eckstein, editor, Oxford University Press, Oxford, pp. 1-24 (1991), and incorporated herein by reference.

An example of a synthetic procedure uses Expedite RNA phosphoramidites and thymidine phosphoramidite (Proligo, Germany). Synthetic oligonucleotides are deprotected and gel-purified (Elbashir et al. (2001) Genes & Dev. 15, 188-200), followed by Sep-Pak C18 cartridge (Waters, Milford, Mass., USA) purification (Tuschl et al. (1993) Biochemistry, 32:11658-11668). Complementary ssRNAs are incubated in an annealing buffer (100 mM potassium acetate, 30 mM HEPES-KOH at pH 7.4, 2 mM magnesium acetate) for 1 min at 90° C. followed by 1 h at 37° C. to hybridize to the corresponding ds-siRNAs.

Other methods of oligonucleotide synthesis include, but are not limited to solid-phase oligonucleotide synthesis according to the phosphotriester and phosphodiester methods (Narang, et al., (1979) Meth. Enzymol. 68:90), and to the H-phosphonate method (Garegg, P. J., et al., (1985) “Formation of internucleotidic bonds via phosphonate intermediates”, Chem. Scripta 25, 280-282; and Froehler, B. C., et al., (1986a) “Synthesis of DNA via deoxynucleoside H-phosphonate intermediates”, Nucleic Acid Res., 14, 5399-5407, among others) and synthesis on a support (Beaucage, et al. (1981) Tetrahedron Letters 22:1859-1862) as well as phosphoramidate techniques (Caruthers, M. H., et al., “Methods in Enzymology,” Vol. 154, pp. 287-314 (1988), U.S. Pat. Nos. 5,153,319, 5,132,418, 4,500,707, 4,458,066, 4,973,679, 4,668,777, and 4,415,732, and others described in “Synthesis and Applications of DNA and RNA,” S. A. Narang, editor, Academic Press, New York, 1987, and the references contained therein, and nonphosphoramidite techniques. Solid phase synthesis helps isolate the oligonucleotide from impurities and excess reagents. Once cleaved from the solid support the oligonucleotide may be further isolated by known techniques.

Inhibitory Polynucleotides of the Invention

A targeting polynucleotide of the invention may be a DNA, an RNA, a mixed DNA-RNA polynucleotide strand, or a DNA-RNA hybrid. An example of the latter is an RNA sequence terminated at the 3′ end with a deoxydinucleotide sequence, such as d(TT), d(UU), d(TU), d(UT), as well as other possible dinucleotides. In additional embodiments the 3′ overhang may be constituted of ribonucleotides having the bases specified above, or others. Furthermore, the oligonucleotide pharmaceutical agent may be single stranded or double stranded. Several embodiments of the therapeutic oligonucleotides of the invention are envisioned to be oligoribonucleotides, or oligoribonucleotides with 3′ d(TT) terminals. A single stranded targeting polynucleotide, if administered into a mammalian cell, is readily converted upon entry to a double stranded molecule by endogenous enzyme activity resident in the cell. The resulting double stranded oligonucleotide triggers RNA interference.

The targeting polynucleotide may be a single stranded polynucleotide or a double stranded polynucleotide. A targeting nucleotide sequence contained within the polynucleotide may be comprised entirely of naturally occurring nucleotides, or at least one nucleotide of the polynucleotide may be a modified nucleotide or a derivatized nucleotide. Modification or derivatization may accomplish objectives such as stabilization of the polynucleotide, optimizing the hybridization of a strand with a complement, or enhancing the induction of the RNAi process. All equivalent polynucleotides that are understood by workers of skill in molecular biology, cell biology, oncology and related fields of medicine, and other fields related to the present invention, to comprise a targeting sequence are within the scope of the present invention.

A polynucleotide of the invention includes a targeting sequence, and is effective to inhibit the growth or replication of cells characteristic of the disease or pathology. The first nucleotide sequence, or targeting sequence, in important embodiments of the invention, may be at least 15 nucleotides (nt) in length, and at most 100 nt. In certain important embodiments, the length may be at most 70 nt. In still more important embodiments, the first nucleotide sequence may be 15 nt, or 16 nt, or 17 nt, or 18 nt, or 19 nt, or 20 nt, or 21 nt, or 22 nt, or 23 nt, or 24 nt, or 25 nt, or 26 nt, or 27 nt, or 28 nt, or 29 nt, or 30 nt in length.

The first targeting nucleotide sequence or its complement is generally at least 80% complementary to the sequence that it is targeting in the target gene. Thus in those embodiments identified in the preceding paragraph in which the target sequence ranges between 15 and 30 nt in length, no more than 3, or 4, or 5 nucleotides may differ from complementarity with the target sequence. In significant embodiments the first nucleotide sequence or its complement is at least 85% complementary, or at least 90% complementary, or at least 95% complementary, or at least 97% complementary, to the target sequence.

The first nucleotide sequence or its complement is sufficiently complementary to its target sequence that it induces the RNA interference phenomenon, thereby promoting cleavage of the target nucleic acid by RNase activity. Any equivalent first nucleotide sequence promoting cleavage of the pathogenic nucleic acid falls within the scope of the present invention.

A short hairpin RNA (shRNA) is contemplated as being comprised in the first polynucleotide of the invention. A shRNA includes a targeting first nucleotide sequence, an intervening loop-forming nucleotide sequence, and a second targeting nucleotide sequence complementary to the first targeting sequence. Without wishing to be bound by theory, it is believed that a polynucleotide comprising a first target sequence, a loop, and a second target sequence complementary to the first loops around to form an intramolecular double stranded “hairpin” structure in which the second complementary sequence hybridizes with the first target sequence. Again, not wishing to be bound by theory, it is believed that the RNAi phenomenon is induced by a double stranded RNA sequence forming a complex with its target sequence. Use of a shRNA affords an optimal means to provide the double stranded targeting polynucleotide effective to silence the targeted gene.

In important embodiments the targeting polynucleotide additionally includes a promoter and/or an enhancer sequence in operable relationship with the first nucleotide sequence, or, in the case of an shRNA, in operable relationship with the entire shRNA construct including the first nucleotide sequence, the loop, and the complementary nucleotide sequence.

Vectors. The present invention provides various vectors that contain one or more first polynucleotides of the invention. By including more than one first polynucleotide the vector carries targeting sequences directed at more than one pathogenic target sequence. The pathogenic target sequences may be directed to the same gene, or to different genes in the cells of a subject suffering from the pathology. Advantageously any vector of the invention includes a promoter, an enhancer, or both, operably linked to the first nucleotide sequence or to the shRNA sequence, respectively.

Methods for preparing the vectors of the invention are widely known in the fields of molecular biology, cell biology, oncology and related fields of medicine, and other fields related to the present invention. Methods useful for preparing the vectors are described, by way on nonlimiting example, in Molecular Cloning: A Laboratory Manual (3^{rd} Edition) (Sambrook, J et al. (2001) Cold Spring Harbor Laboratory Press, Cold Spring Harbor, N.Y.), and Short protocols in molecular biology (5^{th} Ed.) (Ausubel F M et al. (2002) John Wiley & Sons, New York City).

Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′}, and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope of a Target Gene polypeptide encompassed by the antigenic peptide is a region of a polypeptide that is located on the surface of the protein, e.g., a hydrophilic region. A hydrophobicity analysis of the human protein sequence will indicate which regions of a polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice. Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al. In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol. 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-base.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et, al., editors) Mack Pub. Co., Easton, Pa.: 1995; Drug Absorption Enhancement: Concepts, Possibilities, imitations, And Trends, Harwood Academic Publishers, Langhorne, Pa., 1994; and Peptide And Protein Drug Delivery (Advances In Parenteral Sciences, Vol. 4), 1991, M. Dekker, New York.

The present invention includes antibodies binding to Target Gene protein products that can be produced from mouse, rabbit, goat, horse and other mammals. Therapeutic antibodies directed product polypeptides of an ICT-1053 gene, or an ICT-1052 gene, or an ICT-1027 gene, or an ICT-1051 gene, or an ICT-1054 gene, or an ICT-1020 gene, or an ICT-1021 gene, or an ICT-1022 gene include the mouse antibodies, chimeric antibodies, humanized forms and human monoclonal antibodies. Specific monoclonal antibodies directed against a Target Gene product polypeptide are able to increase the apoptosis activity of cancer cells when they were treat with the antibody. Such specific monoclonal antibodies are further able to decrease cancer cell proliferation when they are treated with the antibody. Target Gene specific monoclonal antibodies have potential to inhibit tumor growth in vivo, with both xenograft and Syngenic tumor models.

Cancer Cell Target Polynucleotides of the Invention

Several target genes were identified as lead target genes in the present invention. These target genes are considered validated targets for inhibition of tumor growth, disease progression and methods and compositions for the inhibition and treatment of tumors and cancers in mammals, and in particular, in humans. The validation is based in part on the showings presented in the Examples below of that the Target. Genes are targets for inhibition of tumor growth or promotion of apoptosis, and can thus be used as targets for therapy; and, they also can be used to identify compounds useful in the diagnosis, prevention, and therapy of tumors and cancers. The Target Genes are summarized in Table 1.

TABLE 1
Target Genes

| | | | SEQ ID | SEQ ID |
|---|---|---|---|---|
| | | | NO: for | NO: for |
| Target | | | Polynu- | Encoded |
| Gene | GenBank Acc. Nos. | Characterization | cleotide | Polypeptide |
| ICT-1052 | J02958, NM_000245, | H. sap. c-Met, met proto- | 1 | 2 |
| | NM_008591, AF075090, | oncogene (hepatocyte | | |
| | and X54559 | growth factor (HGF) | | |
| | | receptor) | | |
| ICT-1053 | BC002506, NM_007217, | H. sap. programmed cell | 3 | 4 |
| | BC019650, NM_019745, | death 10 (PDCD10) | | |
| | BC016353, NM_145860, | | | |
| | NM_145859, and | | | |
| | AF022385 | | | |
| ICT-1027 | BC043007, AF171699, | H. sap. growth factor | 5 | 6 |
| | NM_002086, | receptor-bound protein 2, | | |
| | NM_203506, CR450363, | GRB2 | | |
| | CR541942, and M96995 | | | |
| ICT-1051 | L24038, U01337, | H. sap. murine sarcoma | | |
| | Z84466, AB208831, | 3611 viral (v-raf) oncogene | | |
| | AK130043, BC002466, | homolog 1 (ARAF1)), | | |
| | BC007514, BT019864, | (Ser/Thr protein kinase) | | |
| | M13829, X04790 | | | |
| ICT-1054 | BC053586, BC050597, | H. sap. programmed cell | | |
| | BC045555, BC044220, | death 6 | | |
| | BC028242, BC020552, | | | |
| | BC019918, BC014604, | | | |
| | BC012384, | | | |
| | AK223366, AF035606, | | | |
| | AK001917 | | | |
| ICT-1020 | AJ132261, BC046934, | H. sap. hypothetical | | |
| | NM_177438, AB028449 | helicase K12H4.8-like | | |
| | | protein; H. sap. Ortholog | | |
| | | of Drosophila Dicer | | |
| ICT-1021 | BC020690, AB018549, | H. sap. lymphocyte antigen | | |
| | AF168121 | 96, MD-2, ESOP1 | | |
| ICT-1022 | BC069309, BC069397, | H. sap. G antigen 2 | | |
| | BC069558, U19143 | (GAGE-2) | | |

ICT-1052: The target ICT-1052 has been identified as C-MET proto-oncogene (hepatocyte growth factor (HGF) receptor; see Bottaro et al., Science, 251:802-804; Naldini et al, Oncogene, 6: 501-504; Park et al., 1987, Proc. Natl. Acad. USA, 84: 6379-83; WO 92/13097; WO 93/15754; WO 92/20792; Prat et al., 1991, Mol. Cell. Biol., 11:5954-62). The expression of c-Met is detected in various human solid tumors (Prat et al., 1991, Int. J. cancer, 49:323-328) and is implicated in thyroid tumors derived from follicular epithelium (DiRenzo et al., 1992, Oncogene, 7:2549-53). c-Met and its splice variants behaved like a tumor enhancing target since the siRNA-mediated ICT-1052 knockdown resulted in tumor growth inhibition. It is believed that the target ICT-1052 is a tumor stimulator and so is a target for treating tumors, cancers, and precancerous states in mammalian tissues using antibodies, small molecules, antisense, siRNA and other antagonist agents.

Several antibodies to c-Met, including monoclonal antibodies (mAbs), referred to as DL-21, DN-30, DN-31, and DO-24, are specific for the extracellular domain of the 145-kDa β-chain of the c-Met (WO 92/20792; Prat et al., 1991, Mol. Cell. Biol., 11:5954-62) or the intracellular domain (Bottaro et al, Science, 251:802-804). Such antibodies have been used in diagnostic and therapeutic applications (Prat et al., 1991, Int. J. Cancer, 49:323-328; Yamada, et al., 1994, Brain Research, 637:308-312; Crepaldi et al., 1994, J. Cell Biol., 125; 313-20; U.S. Pat. No. 5,686,292; U.S. Pat. No. 6,207,152; patent application of Mark Kay; US Patent Application Publication 20030118587; WO2004/07877 and WO 2004/072117).

The target ICT-1052 includes polymorphic variants, alleles, mutants, and interspecies orthologs that have (i) substantial nucleotide sequence homology (for example, at least 60% identity, preferably at least 70% sequence identity, more preferably at least 80%, still more preferably at least 90% and even more preferably at least 95%) with the nucleotide sequence of the sequence disclosed in the GenBank accession nos. referenced in Table 1, or to its encoded polypeptide. ICT-1052 polynucleotides or polypeptides are typically from a mammal including, but not limited to, human, rat, mouse, hamster, cow, pig, horse, and sheep.

A nucleotide sequence for ICT-1052 contains 6641 base pairs (see SEQ ID NO:1 in the Sequence Listing appended hereto; disclosed in priority document U.S. 60/642,067), encoding a protein of 1390 amino acids (see SEQ ID NO:2 in the Sequence Listing appended hereto; disclosed in priority document U.S. 60/642,067).

ICT-1053: The target designated ICT-1053 is PDCD10, programmed cell death 10 (PDCD10. This gene encodes a protein, originally identified in a premyeloid cell line, with similarity to proteins that participate in apoptosis. PDCD10 protein was able to inhibit the apoptosis of 293 cells in culture (Ma et al. 1998). ICT-1053 is up regulated in fast growing tumors. Inhibition of this target can play an important role in the therapy of various cancer types, tumors and precancerous states. Thus siRNA, monoclonal antibody, and small molecule inhibitors of this target may be useful for cancer treatment using antibodies, small molecules, antisense, siRNA and other antagonist agents.

The target ICT-1053 includes polymorphic variants, alleles, mutants, and interspecies orthologs that have (i) substantial nucleotide sequence homology (for example, at least 60% identity, preferably at least 70% sequence identity, more preferably at least 80%, still more preferably at least 90% and even more preferably at least 95%) with the nucleotide sequence of the sequence disclosed in the GenBank accession nos. referenced in Table 1, or to its encoded polypeptide. ICT-1053 polynucleotides or polypeptides are typically from a mammal including, but not limited to, human, rat, mouse, hamster, cow, pig, horse, and sheep.

A nucleotide sequence for ICT-1053 contains 1466 base pairs (see SEQ ID NO:3 in the Sequence Listing appended hereto; disclosed in priority document U.S. 60/642,067), encoding a protein of 212 amino acids (see SEQ ID NO:4 in the Sequence Listing appended hereto; disclosed in priority document U.S. 60/642,067).

ICT-1027: The target ICT-1027 is Homo sapiens growth factor receptor-bound protein 2, GRB2, having the ability to bind the epidermal growth factor receptor (EGFR) (Lowenstein et al. (1992)). GRB2 gene encodes a protein that has homology to noncatalytic regions of the SRC oncogene product, and is a homolog of the Sem5 gene of C. elegans, which is involved in the signal transduction pathway leading to induction of vulva formation. Drk, the Drosophila homolog of GRB2, plays an essential role in fly photoreceptor development. Various studies have provided evidence for a mammalian GRB2-Ras signaling pathway, mediated by SH2/SH3 domain interactions, that has multiple functions in embryogenesis and cancer. ICT-1027 is up regulated in fast growing tumors. It is believed that the target ICT-1027 is a novel target for treating tumors, cancers, and precancerous states in mammalian tissues using antibodies, small molecules, antisense, siRNA and other antagonist agents.

The target ICT-1027 includes polymorphic variants, alleles, mutants, and interspecies orthologs that have (i) substantial nucleotide sequence homology (for example, at least 60% identity, preferably at least 70% sequence identity, more preferably at least 80%, still more preferably at least 90% and even more preferably at least 95%) with the nucleotide sequence of the sequence disclosed in the GenBank accession nos. referenced in Table 1, or to its encoded polypeptide. ICT-1052 polynucleotides or polypeptides are typically from a mammal including, but not limited to, human, rat, mouse, hamster, cow, pig, horse, and sheep.

A nucleotide sequence for ICT-1027 contains 3317 base pairs (see SEQ ID NO:5 in the Sequence Listing appended hereto; disclosed in priority document U.S. 60/642,067), encoding a protein of 217 amino acids (see SEQ ID NO:6 in the Sequence Listing appended hereto; disclosed in priority document U.S. 60/642,067).

Target ICT-1051. Limiting Apaf-1 activity may alleviate both pathological protein aggregation and neuronal cell death in HD. A-Raf residues are identified that bind to specific phosphoinositides, possibly as a mechanism to localize the enzyme to particular membrane microdomains rich in these phospholipids. The mutation analysis of the conserved regions in the ARAF gene in human colorectal adenocarcinoma has reviewed its role in tumorigenesis. In a two-hybrid screen of human fetal liver cDNA library, TH1 was detected as a new interaction partner of A-Raf; this specific interaction may have played a critical role in the activation of A-Raf. A-Raf kinase is negatively regulated by trihydrophobin 1 and A-Raf interacts with MEK1 and activates MEK1 by phosphorylation.

Target ICT-1054. Raf-1 may mediate its anti-apoptotic function by interrupting ASK1-dependent phosphorylation of ALG-2 (PCDP6). The down-regulation of ALG-2 in human uveal melanoma cells compared to their progenitor cells, normal melanocytes, may provide melanoma cells with a selective advantage by interfering with Ca+-mediated apoptotic signals, thereby enhancing cell survival. Data show that ALG-2 is overexpressed in liver and lung neoplasms, and is mainly found in epithelial cells in the lung. ALG-2 has roles in both cell proliferation and cell death. The penta-EF-hand domain of ALG-2 interacts with amino-terminal domains of both annexin VII and annexin XI in a Ca2+-dependent manner. Pro/Gly/Tyr/Ala-rich hydrophobic region in Anexin XI masked the Ca(2+)-dependently exposed hydrophobic surface of ALG-2. ALG-2 is stabilized by dimerization through its fifth EF-hand region. Apoptosis-linked gene 2 binds to the death domain of Fas and dissociates from Fas during Fas-mediated apoptosis in Jurkat cells.

Target ICT-1020. Various attributes of the 3′ end structure, including overhang length and sequence composition, play a primary role in determining the position of Dicer cleavage in both dsRNA and unimolecular, short hairpin RNA. Dicer is essential for formation of the heterochromatin structure in vertebrate cells. Dicer has a single RNA post-transcriptional processing center. The fragile X syndrome CGG repeats readily form RNA hairpins and is digested by the human Dicer enzyme, a step central to the RNA interference effect on gene expression.

Target ICT-1021. There is evidence to illustrate the function of MD-2 as the primary molecular site of lipopolysaccharide (LPS)-dependent antagonism of Escherichia coli LPS at the Toll-like receptor 4 signaling complex. These results clearly demonstrate that the amino-terminal TLR4 region of Glu(24)-Pro(34) is critical for MD-2 binding and LPS signaling. MD-2 is an important mediator of organ inflammation during sepsis. A rare A to G substitution at position 103, encoding a mutation of Thr 35 to Ala, results in a reduced lipopolysaccharide-induced signaling. Results show that the N-terminal region of toll-like receptor 4 is essential for association with MD-2, which is required for the cell surface expression and hence the responsiveness to lipopolysaccharide. The extracellular toll-like receptor 4 (TLR4)domain-MD-2 complex is capable of binding lipopolysaccharide (LPS) and attenuating LPS-induced NF-kappa B activation and IL-8 secretion in wild-type TLR4-expressing cells. The regulation of MD-2 expression in airway epithelia and pulmonary macrophages may serve as a means to modify endotoxin responsiveness in the airway. MD-2 basic amino acid clusters are involved in cellular lipopolysaccharide recognition TLR4 is able to undergo multiple glycosylations without MD-2 but that the specific glycosylation essential for cell surface expression requires the presence of MD-2. Two functional domains exist in MD-2, one responsible for Toll-like receptor 4-binding and another that mediates the interaction with the agonist (lipopolysaccharide). MD-2 binds to lipopolysaccharide, leading to Toll-like receptor-4 aggregation and signal transduction. Some data support the hypothesis that lipopolysaccharide binding protein can inhibit cell responses to lipopolysaccharide (LPS) by inhibiting LPS transfer from membrane CD14 to the Toll-like receptor 4-MD-2 signaling receptor. Innate immune recognition of LTA via LBP, CD14, and TLR-2 represents an important mechanism in the pathogenesis of systemic complications in the course of infectious diseases brought about by Gram-positive pathogens while TLR-4 and MD-2 are not involved. Disulfide bonds are involved in the assembly and function of this protein. Lipopolysaccharide rapidly traffics to and from the Golgi apparatus with the toll-like receptor 4-MD-2-CD14 complex. Expression regulated by immune-mediated signals in intestinal epithelial cells. MD-2 can confer on mouse Toll-like receptor 4 (TLR4) responsiveness to lipid A but not to lipid IVa, thus influencing the fine specificity of TLR4. Expression of accessory molecule MD-2 is downregulated in intestinal epithelial cells by a mechanism which limits dysregulated immune signaling and activation of proinflammatory genes in response to bacterial lipopolysaccharide. There is no previous report to show that MD-2 is involved in the tumorigenesis.

Target ICT-1022. This gene belongs to a family of genes that are expressed in a variety of tumors but not in normal tissues, except for the testis. The sequences of the family members are highly related but differ by scattered nucleotide substitutions. The antigenic peptide YRPRPRRY, which is also encoded by several other family members, is recognized by autologous cytolytic T lymphocytes. Nothing is presently known about the function of this protein.

It is reported in the Examples below that when the targets ICT-1052, ICT-1053, ICT-1027, ICT-1051, ICT-1054, ICT-1020, ICT-1021 and ICT-1022 were down regulated by two specific siRNA molecules tumor growth rates decreased.

The invention provides broadly for oligonucleotides intended to provoke an RNA interference phenomenon upon entry into a precancerous or cancerous cell. The present invention, while not restricted in the nature of the cancer cell target gene, emphasizes oligonucleotides targeting a Target Gene of the invention. RNA interference is engendered within the cell by appropriate double stranded RNAs one of whose strands has a complement that is identical to or highly similar to a sequence in a target polynucleotide of the cancer cell. In general, an oligonucleotide that targets a Target Gene may be a DNA or an RNA, or it may contain a mixture of ribonucleotides and deoxyribonucleotides. Most generally the invention provides oligonucleotides or polynucleotides that may range in length anywhere from 15 nucleotides to as long as 200 nucleotides. The polynucleotides include a first nucleotide sequence that targets an ICT-1053 gene, or an ICT-1052 gene, or an ICT-1027 gene, or an ICT-1051 gene, or an ICT-1054 gene, or an ICT-1020 gene, or an ICT-1021 gene, or an ICT-1022 gene. The first nucleotide sequence consists of either a) a targeting sequence whose length is any number of nucleotides from 15 to 30, or b) a complement thereof. Such a polynucleotide is termed a linear polynucleotide herein.

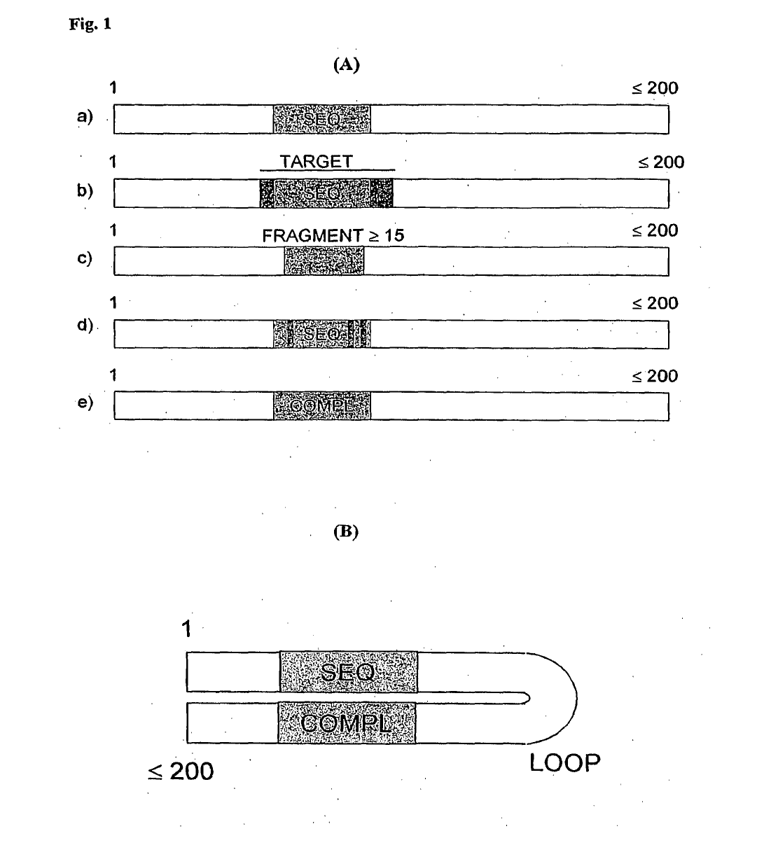

FIG. 1 provides schematic representations of certain embodiments of the polynucleotides of the invention. The invention discloses sequences that target an ICT-1053 gene, or an ICT-1052 gene, or an ICT-1027 gene, or an ICT-1051 gene, or an ICT-1054 gene, or an ICT-1020, gene, or an ICT-1021 gene, or an ICT-1022 gene, or in certain cases siRNA sequences that are slightly mismatched from such a target sequence, all of which are provided in SEQ ID NOS:7-76, 81-84, and 89-242, which are disclosed in Example 1. The sequences disclosed therein range in length from 19 nucleotides to 25 nucleotides. The targeting sequences are represented schematically by the lightly shaded blocks in FIG. 1. FIG. 1, Panel A, a) illustrates an embodiment in which the disclosed sequence shown as “SEQ” may optionally be included in a larger polynucleotide whose overall length may range up to 200 nucleotides.

The invention additionally provides that, in the targeting polynucleotide, a sequence chosen from SEQ ID NOS:7-76, 81-84, and 89-242 may be part of a longer targeting sequence such that the targeting polynucleotide targets a sequence in a target gene that is longer than the first nucleotide sequence represented by SEQ. This is illustrated in FIG. 1, Panel A, b), in which the complete targeting sequence is shown by the horizontal line above the polynucleotide, and by the darker shading surrounding the SEQ block. As in all embodiments of the polynucleotides, this longer sequence may optionally be included in a still larger polynucleotide of length 200 or fewer bases (FIG. 1, Panel A, b)).

The invention further provides a targeting sequence that is a fragment of any of the above targeting sequences such that the fragment targets a sequence given in SEQ ID NOS:7-76, 81-84, and 89-242 that is at least 15 nucleotides in length (and at most 1 base shorter than the reference SEQ ID NO: illustrated in FIG. 1, Panel A, c)), as well as a targeting sequence wherein up to 5 nucleotides may differ from being complementary to the target sequence given in SEQ ID NOS:7-76, 81-84, and 89-242 (illustrated in FIG. 1, Panel A, d), showing, in this example, three variant bases represented by the three darker vertical bars).

Still further the invention provides a sequence that is a complement to any of the above-described sequences (shown in FIG. 1, Panel A, e), and designated as “COMPL”). Any of these sequences are included in the oligonucleotides or polynucleotides of the invention. Any linear polynucleotide of the invention may be constituted of only the sequences described in a)-e) above, or optionally may include additional bases up to the limit of 200 nucleotides. Since RNA interference requires double stranded. RNAs, the targeting polynucleotide itself may be double stranded, including a second strand complementary to at least the sequence given by SEQ ID NOS:7-76, 81-84, and 89-242 and hybridized thereto, or intracellular processes may be relied upon to generate a complementary strand.

Thus a polynucleotide of the invention most generally may be single stranded, or it may be double stranded. In still further embodiments, the polynucleotide contains only deoxyribonucleotides, or it contains only ribonucleotides, or it contains both deoxyribonucleotides and ribonucleotides. In important embodiments of the polynucleotides described herein the target sequence consists of a sequence that may be either 15 nucleotides (nt), or 16 nt, or 17 nt, or 18 nt, or 19 nt, or 20 nt, or 21 nt, or 22 nt, or 23 nt, or 24 nt, or 25 nt, or 26 nt, or 27 nt, or 28 nt, or 29, or 30 nt in length. In still additional advantageous embodiments the targeting sequence may differ by up to 5 bases from complementarity to a target sequence in the viral pathogen genome.

In several embodiments of the invention, the polynucleotide is an siRNA consisting of the targeting sequence with optional inclusion of a 3′ overhang as described herein that may be 1 nt, or 2 nt, or 3 nt, or 4 nt in length; in many embodiments a 3′ overhang is a dinucleotide.

Alternatively, in recognition of the need for a double stranded RNA in RNA interference, the oligonucleotide or polynucleotide may be prepared to form an intramolecular hairpin looped double stranded molecule. Such a molecule is formed of a first sequence described in any of the embodiments of the preceding paragraphs followed by a short loop sequence, which is then followed in turn by a second sequence that is complementary to the first sequence. Such a structure forms the desired intramolecular hairpin. Furthermore, this polynucleotide is disclosed as also having a maximum length of 200 nucleotides, such that the three required structures enumerated may be constituted in any oligonucleotide or polynucleotide having any overall length of up to 200 nucleotides. A hairpin loop polynucleotide is illustrated in FIG. 1, Panel B.

The term “complexed DNA” include a DNA molecule complexed or combined with another molecule, for example, a carbohydrate, for example, a sugar, that a sugar-DNA complex is formed. Such complex, for example, a sugar complexed DNA can enhance or support efficient gene delivery via receptor, for example, glucose can be complexed with DNA and delivered to a cell via receptor, such as mannose receptor.

“Encapsulated nucleic acids”, including encapsulated DNA or encapsulated RNA, refer to nucleic acid molecules in microsphere or microparticle and coated with materials that are relatively non-immunogenic and subject to selective enzymatic degradation, for example, synthesized microspheres or microparticles by the complex coacervation of materials, for example, gelatin and chondroitin sulfate (see, for example, U.S. Pat. No. 6,410,517). Encapsulated nucleic acids in a microsphere or a microparticle are encapsulated in such a way that it retains its ability to induce expression of its coding sequence (see, for example, U.S. Pat. No. 6,406,719).

Pharmaceutical Compositions Comprising Targeting Polynucleotides

Pharmaceutical compositions for therapeutic applications include one or more, targeting polynucleotides and a carrier. The pharmaceutical composition comprising the one or more targeting polynucleotide is useful for treating a disease or disorder associated with the expression or activity of a Target Gene. Carriers include, but are not limited to, saline, buffered saline, dextrose, water, glycerol, ethanol, and combinations thereof. For drugs administered orally, pharmaceutically acceptable carriers include, but are not limited to, pharmaceutically acceptable excipients such as inert diluents, disintegrating agents, binding agents, lubricating agents, sweetening agents, flavoring agents, coloring agents and preservatives.

In many embodiments, the invention relates to a pharmaceutical composition comprising at least two targeting polynucleotides, designed to target different Target Genes, and a pharmaceutically acceptable carrier. Due of the targeting of mRNA of more than one Target Gene, pharmaceutical compositions comprising a plurality of targeting polynucleotides may provide improved efficiency of treatment as compared to compositions comprising a single targeting polynucleotide, at least in tumor cells expressing these multiple genes. In this embodiment, the individual targeting polynucleotides are prepared as described in the preceding section, which is incorporated by reference herein. One targeting polynucleotide can have a nucleotide sequence which is substantially complementary to at least part of one Target Gene; additional targeting polynucleotides are prepared, each of which has a nucleotide sequence that is substantially complementary to part of a different Target Gene. The multiple targeting polynucleotides may be combined in the same pharmaceutical composition, or formulated separately. If formulated individually, the compositions containing the separate targeting polynucleotides may comprise the same or different carriers, and may be administered using the same or different routes of administration. Moreover, the pharmaceutical compositions comprising the individual targeting polynucleotides may be administered substantially simultaneously, sequentially, or at preset intervals throughout the day or treatment period.

The pharmaceutical compositions of the present invention are administered in dosages sufficient to inhibit expression of the target gene. The targeting polynucleotides are highly efficient in producing an inhibitory effect, as it is understood that, as part of a RISC complex, they act in a catalytic fashion. Thus compositions comprising the one or more targeting polynucleotides of the invention can be administered at surprisingly low dosages.

A maximum dosage of 5 mg targeting polynucleotide per kilogram body weight of recipient per day is sufficient to inhibit or completely suppress expression of the target gene. In general, a suitable dose of targeting polynucleotide will be in the range of 0.01 to 5.0 milligrams per kilogram body weight of the recipient per day, preferably in the range of 0.1 to 200 micrograms per kilogram body weight (mcg/kg) per day, more preferably in the range of 0.1 to 100 mcg/kg per day, even more preferably in the range of 1.0 to 50 mcg/kg per day, and most preferably in the range of 1.0 to 25 mcg/kg per day. The pharmaceutical composition may be administered once daily, or the targeting polynucleotide may be administered as two, three, four, five, six or more sub-doses at appropriate intervals throughout the day. In that case, the targeting polynucleotide contained in each sub-dose must be correspondingly smaller in order to achieve the total daily dosage. The dosage unit can also be compounded as a sustained release formulation for delivery over several days, e.g., using a conventional formulation which provides sustained release of the targeting polynucleotide over a several day period. Sustained release formulations are well known in the art. In this embodiment, the dosage unit contains a corresponding multiple of the daily dose.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual targeting polynucleotides encompassed by the invention can be made using conventional methodologies or on the basis of in vivo testing using an appropriate animal model, and can be adjusted during treatment according to established criteria for determining appropriate dose-response characteristics.

Advances in mouse genetics have generated a number of mouse models for the study of various human diseases. For example, mouse models are available for hematopoietic malignancies such as leukemias, lymphomas and acute myelogenous leukemia. The MMHCC (Mouse models of Human Cancer Consortium) web page (emice.nci.nih.gov), sponsored by the National Cancer Institute, provides disease-site-specific compendium of known cancer models, and has links to the searchable Cancer Models Database (cancermodels.nci.nih.gov), as well as the NCI-MMHCC mouse repository. Examples of the genetic tools that are currently available for the modeling of leukemia and lymphomas in mice, and which are useful in practicing the present invention, are described in the following references: Maru, Y., Int. J. Hematol. (2001) 73:308-322; Pandolfi, P. P., Oncogene (2001) 20:5726-5735; Pollock, J. L., et al., Curr. Opin. Hematol. (2001). delta: 206-211; Rego, E. M., et al., Semin. in Hemat. (2001) 38:4-70; Shannon, K. M., et al. (2001) Modeling myeloid leukemia tumors suppressor gene inactivation in the mouse, Semin. Cancer Biol. 11, 191-200; Van Etten, R. A, (2001) Curr. Opin. Hematol. 8, 224-230; Wong, S., et al. (2001) Oncogene 20, 5644-5659; Phillips J A., Cancer Res. (2000) 52(2): 437-43; Harris, A. W., et al, J. Exp. Med. (1988) 167(2): 353-71; Zeng X X et al., Blood. (1988) 92(10): 3529-36; Eriksson, B., et al., Exp. Hematol. (1999) 27(4): 682-8; and Kovalchuk, A., et al., J. Exp. Med. (2000) 192(8): 1183-90. Mouse repositories can also be found at: The Jackson Laboratory, Charles River Laboratories, Taconic, Harlan, Mutant Mouse Regional Resource Centers (MMRRC) National Network and at the European Mouse Mutant Archive. Such models may be used for in vivo testing of targeting polynucleotide, as well as for determining a therapeutically effective dose. Furthermore various knock-out or knock-in transgenic animal models for effects of the Target Genes can be prepared and studied to evaluate dosing of targeting polynucleotides.

The pharmaceutical compositions encompassed by the invention may be administered by any means known in the art including, but not limited to oral or parenteral routes, including intravenous, intramuscular, intraperitoneal, subcutaneous, transdermal, airway (aerosol), rectal, vaginal and topical (including buccal and sublingual) administration. In certain embodiments, the pharmaceutical compositions are administered by intravenous or intraparenteral infusion or injection, and in additional common embodiments the pharmaceutical composition comprising targeting polynucleotides may be delivered directly in situ to a tumor, a cancer or a precancerous growth using laparoscopic and similar microsurgical procedures.

For intramuscular, intraperitoneal, subcutaneous and intravenous use, the pharmaceutical compositions of the invention will generally be provided in sterile aqueous solutions or suspensions, buffered to an appropriate pH and isotonicity. Suitable aqueous vehicles include Ringer's solution and isotonic sodium chloride. In a preferred embodiment, the carrier consists exclusively of an aqueous buffer. In this context, “exclusively” means no auxiliary agents or encapsulating substances are present which might affect or mediate uptake of targeting polynucleotide in the cells that express the target gene. Such substances include, for example, micellar structures, such as liposomes or capsids, as described below. Surprisingly, the present inventors have discovered that compositions containing only naked targeting polynucleotide and a physiologically, acceptable solvent are taken up by cells, where the targeting polynucleotide effectively inhibits expression of the target gene. Although microinjection, lipofection, viruses, viroids, capsids, capsoids, or other auxiliary agents are required to introduce targeting polynucleotide into cell cultures, surprisingly these methods and agents are not necessary for uptake of targeting polynucleotide in vivo. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The pharmaceutical compositions useful according to the invention also include encapsulated formulations to protect the targeting polynucleotide against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP-A-43075, which are incorporated by reference herein.

In certain embodiments, the encapsulated formulation comprises a viral coat protein. In this embodiment, the targeting polynucleotide may be bound to, associated with, or enclosed by at least one viral coat protein. The viral coat protein may be derived from or associated with a virus, such as a polyoma virus, or it may be partially or entirely artificial. For example, the coat protein may be a Virus Protein 1 and/or Virus Protein 2 of the polyoma virus, or a derivative thereof.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred.

The data obtained from cell culture assays and animal studies can be used in formulation a range of dosage for use in humans. The dosage of compositions of the invention lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays and animal models to achieve a circulating plasma concentration range of the compound that includes the IC50 (i.e., the concentration of the test compound which achieves a half-maximal inhibition of symptoms) as determined in cell culture. Such information can be used to more accurately determine useful doses in humans.

In addition to their administration individually or as a plurality, as discussed above, the targeting polynucleotides useful according to the invention can be administered in combination with other known agents effective in treatment of diseases. In any event, the administering physician can adjust the amount and timing of targeting polynucleotide administration on the basis of results observed using standard measures of efficacy known in the art or described herein.

It is further envisioned to use Intradigm Corporation's proprietary gene delivery technologies for high throughput delivery into animal models. Intradigm's PolyTran™ technology (see International Application No. WO 0147496) enables direct administration of plasmids into tumor and achieves a seven-fold increase of efficiency over the gold standard nucleotide delivery reagents. This provides strong tumor expression and activity of candidate target proteins in the tumor.

Methods for Treating Diseases Caused by Expression of a Target Gene

In certain embodiments, the invention relates to methods for treating a subject having a disease or at risk of developing a disease caused by the expression of a Target Gene. The one or more targeting polynucleotides can act as novel therapeutic agents for controlling one or more of cellular proliferative and/or differentiative disorders including a tumor, a cancer, or a precancerous growth. The method comprises administering a pharmaceutical composition of targeting polynucleotides to the patient (e.g., human), such that expression of the target gene is silenced. Because of their high specificity, the targeting polynucleotides of the present invention specifically target mRNAs of target genes of diseased cells and tissues, as described below, and at surprisingly low dosages.

In the prevention of disease, the target gene may be one which is required for initiation or maintenance of the disease, or which has been identified as being associated with a higher risk of contracting the disease. In the treatment of disease, the targeting polynucleotide can be brought into contact with the cells or tissue exhibiting the disease. For example, targeting polynucleotide comprising a sequence substantially complementary to all or part of an mRNA formed in the transcription of a mutated gene associated with cancer, or one expressed at high levels in tumor cells may be brought into contact with or introduced into a cancerous cell or tumor.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of pancreas, prostate, colon, lung, breast and liver origin. As used herein, the terms “cancer,” “hyperproliferative,” and “neoplastic” refer to cells having the capacity for autonomous growth, i.e., an abnormal state of condition characterized by rapidly proliferating cell growth. These terms are meant to include all types of cancerous growths or oncogenic processes, metastatic tissues or malignantly transformed cells, tissues, or organs, irrespective of histopathologic type or stage of invasiveness. Proliferative disorders also include hematopoietic neoplastic disorders, including diseases involving hyperplastic/neoplastic cells of hematopoietic origin, e.g., arising from myeloid, lymphoid or erythroid lineages, or precursor cells thereof.

Combinations of siRNA

Several embodiments of the invention provide pharmaceutical compositions containing two or more oligonucleotides or polynucleotides each of which includes a sequence targeting genes in the genome of a respiratory virus. Related embodiments provide methods of treating cells, and methods of treating respiratory viral infections, using the combinations, as well as uses of such combination compositions in the manufacture of pharmaceutical compositions intended to treat respiratory viral infections. The individual polynucleotide components of the combination may target different portions of the same gene, or different genes, or several portions of one gene as well as more than one gene, in the genome of the viral pathogen. An advantage of using a combination of oligonucleotides or polynucleotides is that the benefits of inhibiting expression of a given gene are multiplied in the combination. Greater efficacy is achieved in knocking down a gene or silencing a viral genome by use of multiple targeting sequences. Enhanced efficiency in inhibiting viral replication is achieved by targeting more than one gene in the viral genome.

Pharmaceutical Compositions

The targeting polynucleotides of the invention are designated “active compounds” or “therapeutics” herein. These therapeutics can be incorporated into pharmaceutical compositions suitable for administration to a subject.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in textbooks such as Remington's Pharmaceutical Sciences, Gennaro AR (Ed.) 20^{th} edition (2000) Williams & Wilkins Pa., USA, and Wilson and Gisvold's Textbook of Organic Medicinal and Pharmaceutical Chemistry, by Delgado and Remers, Lippincott-Raven, which are incorporated herein by reference. Preferred examples of components that may be used in such carriers or diluents include, but are not limited to, water, saline, phosphate salts, carboxylate salts, amino acid solutions, Ringer's solutions, dextrose (a synonym for glucose) solution, and 5% human serum albumin. By way of nonlimiting example, dextrose may used as 5% or 10% aqueous solutions. Liposomes and non-aqueous vehicles such as fixed oils may also be used. The use of such media and agents for pharmaceutically active substances is well known in the art. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral, nasal, inhalation, transdermal (topical), transmucosal, and rectal administration. Solutions or suspensions used for parenteral, intravenous, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose.

For administration by inhalation, the compounds are delivered in the form of an aerosol spray from pressured container or dispenser which contains a suitable propellant, e.g., a gas such as carbon dioxide, or a nebulizer.

In one embodiment, the active compounds are prepared with carriers that will protect the compound against rapid elimination from the body, such as a controlled release formulation, including implants and microencapsulated delivery systems. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release pharmaceutical active agents over shorter time periods. Advantageous polymers are biodegradable, or biocompatible. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811. Sustained-release preparations having advantageous forms, such as microspheres, can be prepared from materials such as those described above.

The siRNA polynucleotides of the invention can be inserted into vectors and used as gene therapy vectors. Gene therapy vectors can be delivered to a subject by any of a number of routes, e.g., as described in U.S. Pat. Nos. 5,703,055. Delivery can thus also include, e.g., intravenous injection, local administration (see U.S. Pat. No. 5,328,470) or stereotactic injection (see e.g.; Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a kit, e.g., in a container, pack, or dispenser together with instructions for administration.

Also within the invention is the use of a therapeutic in the manufacture of a pharmaceutical composition or medicament for treating a respiratory viral infection in a subject.

Delivery

In several embodiments the siRNA polynucleotides of the invention are delivered by liposome-mediated transfection, for example by using commercially available reagents or techniques, e.g., Oligofectamine™, LipofectAmine™ reagent, LipofectAmine 2000™ (Invitrogen), as well as by electroporation, and similar techniques. Additionally siRNA polynucleotides are, is delivered to animal models, such as rodents or non-human primates, through inhalation and instillation into the respiratory tract. Additional routes for use with animal models include intravenous (IV), subcutaneous (SC), and related routes of administration. The pharmaceutical compositions containing the siRNAs include additional components that protect the stability of siRNA, prolong siRNA lifetime, potentiate siRNA function, or target siRNA to specific tissues/cells. These include a variety of biodegradable polymers, cationic polymers (such as polyethyleneimine), cationic copolypeptides such as histidine-lysine (HK) polypeptides see, for example, PCT publications WO 01/47496 to Mixson et al., WO 02/096941 to Biomerieux, and WO 99/42091 to Massachusetts Institute of Technology), PEGylated cationic polypeptides, and ligand-incorporated polymers, etc. positively charged polypeptides, PolyTran polymers (natural polysaccharides, also known as scleroglucan), a nano-particle consists of conjugated polymers with targeting ligand (TargeTran variants), surfactants (Infasurf; Forest Laboratories, Inc.; ONY Inc.), and cationic polymers (such as polyethyleneimine). Infasurf® (calfactant) is a natural lung surfactant isolated from calf lung for use in intratracheal instillation; it contains phospholipids, neutral lipids, and hydrophobic surfactant-associated proteins B and C. The polymers can either be uni-dimensional or multi-dimensional, and also could be microparticles or nanoparticles with diameters less than 20 microns, between 20 and 100 microns, or above 100 micron. The said polymers could carry ligand molecules specific for receptors or molecules of special tissues or cells, thus be used for targeted delivery of siRNAs. The siRNA polynucleotides are also delivered by cationic liposome based carriers, such as DOTAP, DOTAP/Cholesterol (Qbiogene, Inc.) and other types of lipid aqueous solutions. In addition, low percentage (5-10%) glucose aqueous solution, and Infasurf are effective carriers for airway delivery of siRNA³⁰.

Using fluorescence-labeled siRNA suspended in an oral-tracheal delivery solution of 5% glucose and Infasurf examined by fluorescence microscopy, it has been shown that after siRNA is delivered to mice via the nostril or via the oral-tracheal route, and washing the lung tissues the siRNA is widely distributed in the lung (see co-owned WO 2005/01940, incorporated by reference herein in its entirety). The delivery of siRNA into the nasal passage and lung (upper and deeper respiratory, tract) of mice was shown to successfully silence the indicator genes (GFP or luciferase) delivered simultaneously with the siRNA in a plasmid harboring a fusion of the indicator gene and the siRNA target (see co-owned WO 2005/01940). In addition, experiments reported by the inventors, working with others, have demonstrated that siRNA species inhibit the replication of SARS coronavirus, thus relieving the lung pathology, in the SARS-infected rhesus monkeys³⁰.

siRNA Recombinant Vectors

Another aspect of the invention pertains to vectors, preferably expression vectors, containing an siRNA polynucleotide of the invention. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention, in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel (1990) GENE EXPRESSION TECHNOLOGY: METHODS IN ENZYMOLOGY 185, Academic Press, San Diego, Calif. Regulatory sequences include those that direct constitutive expression of a nucleotide sequence in many types of host cell and those that direct expression of the nucleotide sequence only in certain host cells (e.g., tissue-specific regulatory sequences). In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J6: 187-195). When used in mammalian cells, the expression vector's control functions are often provided by viral regulatory elements. For example, commonly used promoters are derived from polyoma, Adenovirus 2, cytomegalovirus and Simian Virus 40. Additional vectors include minichromosomes such as bacterial artificial chromosomes, yeast artificial chromosomes, or mammalian artificial chromosomes. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type such as a cell of the respiratory tract. Tissue-specific regulatory elements are known in the art. The invention further provides a recombinant expression vector comprising a DNA molecule of the invention cloned into the expression vector. The DNA molecule is operatively linked to a regulatory sequence in a manner that allows for expression (by transcription of the DNA molecule) of an RNA molecule that includes an siRNA targeting a viral RNA. Regulatory sequences operatively linked to a nucleic acid can be chosen that direct the continuous expression of the RNA molecule in a variety of cell types, for instance viral promoters and/or enhancers, or regulatory sequences can be chosen that direct constitutive, tissue specific or cell type specific expression of antisense RNA.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (2001), Ausubel et al. (2002), and other laboratory manuals.

Method of Treatment

The present invention relates to a method for treating a disease in a mammal associated with pathological expression of an ICT-1053 gene, or an ICT-1052 gene, or an ICT-1027 gene, or an ICT-1051 gene, or an ICT-1054 gene, or an ICT-1020 gene, or an ICT-1021 gene, or an ICT-1022 gene. The method includes administering to the mammal inhibitory nucleic acid compositions that interact with at least one of the targets an ICT-1053 gene, or an ICT-1052 gene, or an ICT-1027 gene, or an ICT-1051 gene, or an ICT-1054 gene, or an ICT-1020 gene, or an ICT-1021 gene, or an ICT-1022 gene at the DNA or RNA level. The nucleic acid composition is capable of suppressing the expression of the one or more targets an ICT-1053 gene, or an ICT-1052 gene, or an ICT-1027 gene, or an ICT-1051 gene, or an ICT-1054 gene, or an ICT-1020 gene, or an ICT-1021 gene, or an ICT-1022 gene when introduced into a tissue of the mammal. The method of treatment is directed in particular to a disease such as a cancer or a precancerous growth in the tissue of the mammal. Frequently the tissue is a breast tissue, a colon tissue, a prostate tissue, a skin tissue, a bone tissue, a parotid gland tissue, a pancreatic tissue, a kidney tissue, a uterine cervix tissue, a lymph node tissue, or an ovarian tissue. In common cases the inhibitor is a siRNA, an RNAi, a shRNA, an antisense RNA, an antisense DNA, a decoy molecule, a decoy DNA, a double stranded DNA, a single-stranded DNA, a complexed DNA, an encapsulated DNA, a viral DNA, a plasmid DNA, a naked RNA, an encapsulated RNA, a viral RNA, a double stranded RNA, a molecule capable of generating RNA interference, or combinations thereof.

The following Examples illustrate certain embodiments of the present invention. They should not be viewed as limiting the scope of the invention, which is represented in the specification as a whole, and, in the claims.

### EXAMPLES

Novel target genes for application of RNA interference to the treatment of cancer were identified, and experiments to assess the tumor inhibition properties of siRNAs directed at the targets were carried out. Specifically, experiments were done by targeting ICT-1053, ICT-1052, ICT-1027, ICT-1051, ICT-1054, ICT-1020, ICT-1021, ICT-1022, and ICT-1022.

Two siRNA target sequences were selected within each gene and verified by BLAST, and the sequences synthesized by Qiagen Inc (Germantown, Md.). In the experiments reported in these Examples, a mixture of two specific siRNA sequences for each gene was repeatedly delivered to xenograft models or to cells in culture. Human VEGF siRNA was used as a positive control against which to assess the effects of the chosen siRNAs.

### Example 1

### Small Interfering RNA (siRNA)

siRNAs duplexes were made based upon selected targeted regions of the DNA sequences for targets ICT-1052, ICT-1053 or ICT-1027 (SEQ ID NOS:1, 3, and 5), or ICT-1051, ICT-1054, ICT-1020, ICT-1021, ICT-1022, or ICT-1022. In certain embodiments a designed sequence includes AA-(N)ₘ-TT (where 15≦m≦21) and has a G-C content of about 30% to 70%. If no suitable sequences are found, the fragment size is extended to sequences of up to 29 nucleotides. In certain embodiments the 3′ end of a polynucleotide has an overhang (i.e. having unpaired bases) given by TT or UU. Without wishing to be bound by theory, it is believed that symmetric 3′ overhangs on an siRNA duplex help to ensure that the small interfering ribonucleoprotein particles (siRNPs) are formed with approximately equal ratios of sense and antisense target RNA-cleaving siRNPs (Elbashir et al. Genes & Dev. 15:188-200, 2001).

ICT-1052 siRNA: Sense or antisense siRNAs of 21 bp were identified based upon targeted regions of SEQ ID NO:1). These are shown in Table 2.

TABLE 2
siRNA targeted sequences identified in ICT-1052)

| TARGET SEQUENCE | SEQ ID NO: |
|---|---|
| AACACCCATCCAGAATGTCAT | 7 |
| AAGCCAATTTATCAGGAGGTG | 8 |
| TCAAGAGCATGAACGCATCAA | 9 |
| TGTGTGTTGTATGGTCAATAA | 10 |
| ACTGAATGGTACTTCGTATGT | 11 |
| CCTCGCAAGCAATTGGAAACA | 12 |
| CTCTGATAGTGCAGAGACTTA | 13 |
| GAATGATGCTACTCTGATCTA | 14 |
| GCAATACAGTCAAAGTTTCAA | 15 |
| TTGTGTGTTGTATGGTCAATA | 16 |
| TTTGTGTGTTGTATGGTCAAT | 17 |
| AGGACTACACACTTGTATATA | 18 |
| AGAGTATTGTAAATGGTGGAT | 19 |
| GAATGGTACTTCGTATGTTAA | 20 |
| CTGTAAATTGCGATAAGGAAA | 21 |
| CCAAATATTGCCGTTTCATAA | 22 |
| CAAGAGCATGAACGCATCAAT | 23 |
| GCATGAACGCATCAATAGAAA | 24 |
| GAAGAGCTATTACAATCCAAA | 25 |
| CATTACATCATCAGGACTTGA | 26 |
| ACAGGACTACACACTTGTATA | 27 |
| CAGGACTACACACTTGTATAT | 28 |

ICT-1053 siRNA: Sense or antisense siRNAs of 21 bp were identified based upon targeted regions of SEQ ID NO:3). These are shown in Table 3.

TABLE 3
siRNA targeted sequences identified in ICT-1053.

| TARGET SEQUENCE | SEQ ID NO: |
|---|---|
| AATCTGTCTGCAGCCCAGAAC | 29 |
| AAGCGTGGAAGTTAACTTCAC | 30 |
| AGTCATGTATCCTGTGTTTAA | 31 |
| GGATATAGCTAGTGCAATAAA | 32 |
| ACGCCTTAATGTGTCATTATA | 33 |
| TTAAAGATGGCAAGGCAATAA | 34 |
| CCGCTTTCATCAAGGCTGAAA | 35 |
| TCGTAAGTGCCAACCGACTAA | 36 |
| AGCGATATGCTGCAAGATAAT | 37 |
| ACTTAATACTTCAGACCTTCA | 38 |
| CAGTCATGTATCCTGTGTTTA | 39 |
| CTTAATACTTCAGACCTTCAA | 40 |
| CTGATGATGTAGAAGAGTATA | 41 |
| TGAACTGCCTTTATCTGTAAA | 42 |
| ACGGATTCAGTTCCAGTTTAA | 43 |
| TTTAAAGATGGCAAGGCAATA | 44 |
| TTAATGAGCTAGAACGAGTAA | 45 |
| TGAAAGCGATATGCTGCAAGA | 46 |
| GAAAGCGATATGCTGCAAGAT | 47 |
| CTTGAAAGCGATATGCTGCAA | 48 |
| TCATAATCTCACACTGAAGAT | 49 |
| TATTGCCATCTTACACCATAT | 50 |

ICT-1027 siRNA: Sense or antisense siRNAs of 21 bp were identified based upon targeted regions of SEQ ID NO:5). These are shown in Table 4.

TABLE 4
siRNA targeted sequences identified in ICT-1027.

| TARGET SEQUENCE | SEQ ID NO: |
|---|---|
| AATCCCCAGAGCCAAGGCAGA | 51 |
| AAGGGGGGACATCCTCAAGGT | 52 |
| AGTCCTAGCTGACGCCAATAA | 53 |
| GGTAGTGATTAACTGTGAATA | 54 |
| CTCCAGTTGTAGCAGGTTTCA | 55 |
| TTCCTGTGTTCTTCGTATATA | 56 |
| TCCATCAGTGCATGACGTTTA | 57 |
| CCTGTGGTGATGTGCCTGTAA | 58 |
| GGAACGTCTAAGAGTCAAGAA | 59 |
| AGAAGAAATGCTTAGCAAACA | 60 |
| TAGTCCTAGCTGACGCCAATA | 61 |
| TGACGTTTAAGGCCACGTATA | 62 |
| CATGAAGCCTTGCTGAACTAA | 63 |
| GTCTCCAGAAACCAGCAGATA | 64 |
| GTTCCTGTGTTCTTCGTATAT | 65 |
| CATTTGGTAGGTAGTGATTAA | 66 |
| GCTCGATGCCTTTGCTGTTTA | 67 |
| CTGTGGTGATGTGCCTGTAAT | 68 |
| GCATTTGGTAGGTAGTGATTA | 69 |
| TCAGCCAATTTGTCTCCTACT | 70 |
| ATATCATGAAGCCTTGCTGAA | 71 |
| TACTAAGCCAGGAGGCTTTAA | 72 |

Additional targeted sequences are identified in Tables 5-18.

TABLE 5
19-nt siRNA targeted sequences identified in
ICT-1053.

| TARGET SEQUENCE | SEQ ID NO: |
|---|---|
| GGCAGCTGATGATGTAGAA | 113 |
| GCCAGAATTCCAAGACCTA | 114 |
| CCAGAATTCCAAGACCTAA | 115 |
| GGCACGAGCACTTAAACAA | 116 |
| GCACGAGCACTTAAACAAA | 117 |
| GGTTTCTGCAGACAATCAA | 118 |
| GCAGACAATCAAGGATATA | 119 |
| CCTCCTGAAGGGATCTAAT | 120 |
| GGATCTAATCCAGGATGTT | 121 |
| GGATGTTGAATGGGATTAT | 122 |

TABLE 6
25-nt siRNA targeted sequences identified in
ICT-1053.

| TARGET SEQUENCE | SEQ ID NO: |
|---|---|
| CCTTCTTCGTATGGCAGCTGATGAT | 123 |
| CAGAGCCAGAATTCCAAGACCTAAA | 124 |
| CCAGAATTCCAAGACCTAAACGAAA | 125 |
| GACAATCAAGGATATAGCTAGTGCA | 126 |
| GGCAAGGCAATAAATGTGTTCGTAA | 127 |
| TCGTAAGTGCCAACCGACTAATTCA | 128 |
| CCGACTAATTCATCAAACCAACTTA | 129 |
| TCAGTCCCTCCTGAAGGGATCTAAT | 130 |
| GAAGGGATCTAATCCAGGATGTTGA | 131 |
| GGGATTATTGCCATCTTACACCATA | 132 |

TABLE 7
19-nt siRNA targeted sequences identified in
ICT-1052.

| TARGET SEQUENCE | SEQ ID NO: |
|---|---|
| CCGGTTCATCAACTTCTTT | 133 |
| GGACCAGTCCTACATTGAT | 134 |
| GCACAAAGCAAGCCAGATT | 135 |
| GCATGTCAACATCGCTCTA | 136 |
| GCTGGTGTTGTCTCAATAT | 137 |
| GGTGTTGTCTCAATATCAA | 138 |
| GCAGTGAATTAGTTCGCTA | 139 |
| CCAACTACAGAAATGGTTT | 140 |
| CCATGTGAACGCTACTTAT | 141 |
| GCATCAGAACCAGAGGCTT | 142 |

TABLE 8
25-nt siRNA targeted sequences identified in
ICT-1052.

| TARGET SEQUENCE | SEQ ID NO: |
|---|---|
| CAAAGCCAATTTATCAGGAGGTGTT | 143 |
| CAGTCGGAGGTTCACTGCATATTCT | 144 |
| CACACAAGAATAATCAGGTTCTGTT | 145 |
| CGCTCTAATTCAGAGATAATCTGTT | 146 |
| CAGCACTGTTATTACTACTTGGGTT | 147 |
| CCAGTAGCCTGATTGTGCATTTCAA | 148 |
| CAGCCTCCTTCTGGGAGACATCATA | 149 |
| TGGGAGACATCATAGTGCTAGTACT | 150 |
| GCAGGAAATATTGAGGGCTTCTTGA | 151 |
| GCCACTCATTTAGAATTCTAGTGTT | 152 |

TABLE 9
19-nt siRNA targeted sequences identified
in ICT-1027.

| TARGET SEQUENCE | SEQ ID NO: |
|---|---|
| CCGGTTCATCAACTTCTTT | 153 |
| GGACCAGTCCTACATTGAT | 154 |
| GCACAAAGCAAGCCAGATT | 155 |
| GCATGTCAACATCGCTCTA | 156 |
| GCTGGTGTTGTCTCAATAT | 157 |
| GGTGTTGTCTCAATATCAA | 158 |
| GCAGTGAATTAGTTCGCTA | 159 |
| CCAACTACAGAAATGGTTT | 160 |
| CCATGTGAACGCTACTTAT | 161 |
| GCATCAGAACCAGAGGCTT | 162 |

TABLE 10
25-nt siRNA targeted sequences identified
in ICT-1027.

| TARGET SEQUENCE | SEQ ID NO: |
|---|---|
| CAAAGCCAATTTATCAGGAGGTGTT | 163 |
| CAGTCGGAGGTTCACTGCATATTCT | 164 |
| CACACAAGAATAATCAGGTTCTGTT | 165 |
| CGCTCTAATTCAGAGATAATCTGTT | 166 |
| CAGCACTGTTATTACTACTTGGGTT | 167 |
| CCAGTAGCCTGATTGTGCATTTCAA | 168 |
| CAGCCTCCTTCTGGGAGACATCATA | 169 |
| TGGGAGACATCATAGTGCTAGTACT | 170 |
| GCAGGAAATATTGAGGGCTTCTTGA | 171 |
| GCCACTCATTTAGAATTCTAGTGTT | 172 |

TABLE 11
19-nt siRNA targeted sequences identified
in ICT-1051.

| TARGET SEQUENCE | SEQ ID NO: |
|---|---|
| GGACTCTGTGAGGAAACAA | 173 |
| GCTTCCAGTCAGACGTCTA | 174 |
| CCACCAGCCAATCAATGTT | 175 |
| CCAATCAATGTTCGTCTCT | 176 |
| TCTCCAATGGCTGGGATTT | 177 |
| GGGATTTGTGGCAGGGATT | 178 |
| GCAGGGATTCCACTCAGAA | 179 |
| GCCATTCAAGGACTCCTCT | 180 |
| TCCTCTCTTTCTTCACCAA | 181 |
| TCTCTTTCTTCACCAAGAA | 182 |

TABLE 12
25-nt siRNA targeted sequences identified
in ICT-1051.

| TARGET SEQUENCE | SEQ ID NO: |
|---|---|
| GGCGGACTCTGTGAGGAAACAAGAA | 183 |
| GGGTTGTGCTCTACGAGCTTATGAC | 184 |
| GCTCTACGAGCTTATGACTGGCTCA | 185 |
| CACAATTGAGCTGCTGCAACGGTCA | 186 |
| CCTTGCCCACCAGCCAATCAATGTT | 187 |
| ACCAGCCAATCAATGTTCGTCTCTG | 188 |
| CCATCTCCAATGGCTGGGATTTGTG | 189 |
| CCGCCATTCAAGGACTCCTCTCTTT | 190 |
| GCCATTCAAGGACTCCTCTCTTTCT | 191 |
| GGACTCCTCTCTTTCTTCACCAAGA | 192 |

TABLE 13
19-nt siRNA targeted sequences identified
in ICT-1054.

| TARGET SEQUENCE | SEQ ID NO: |
|---|---|
| GGCCTGAGAGGTCTCTCGT | 193 |
| GCCTGAGAGGTCTCTCGTC | 194 |
| GAGAGGTCTCTCGTCGCTG | 195 |
| CCCATGGCCGCCTACTCTT | 196 |
| CCATGGCCGCCTACTCTTA | 197 |
| GCTCTCAGGGAGGTCTGTG | 198 |

TABLE 14
19-nt siRNA targeted sequences identified
in ICT-1054.

| TARGET SEQUENCE | SEQ ID NO: |
|---|---|
| GGAGTCGGCCTGAGAGGTCTCTCGT | 199 |
| GAGTCGGCCTGAGAGGTCTCTCGTC | 200 |
| TCGGCCTGAGAGGTCTCTCGTCGCT | 201 |
| CCTTGGCCCATGGCCGCCTACTCTT | 202 |

TABLE 15
19-nt siRNA targeted sequences identified
in ICT-1020.

| TARGET SEQUENCE | SEQ ID NO: |
|---|---|
| GCTCGAAATCTTACGCAAA | 203 |
| GCTTATATCAGTAGCAATT | 204 |
| GCACCCATCTCTAATTATA | 205 |
| GCACTAGAATTTAAACCTA | 206 |
| GCCGTTATCATTCCAAGAT | 207 |
| CCACACATCTTCAAGACTT | 208 |
| GCACATCAAGGTGCTAATA | 209 |
| GCAATTAATGGTCTTTCTT | 210 |
| GCAGTTATGATTTAGCTAA | 211 |
| GCAACCAACTACCTCATAT | 212 |

TABLE 16
25-nt siRNA targeted sequences identified
in ICT-1020.

| TARGET SEQUENCE | SEQ ID NO: |
|---|---|
| CCTGAAATTTGTAACTCCTAAAGTA | 213 |
| GCAGTTGTCTTAAACAGATTGATAA | 214 |
| CAACCTGCTTATTGCAACAAGTATT | 215 |
| CATCAATAGATACTGTGCTAGATTA | 216 |
| TCCAGAGTGTTTGAGGGATAGTTAT | 217 |
| CCTTTACCTGATGAACTCAACTTTA | 218 |
| CAGCATACTGTGTTCTACCTCTTAA | 219 |
| CCAAATGGGAAAGTCTGCAGAATAA | 220 |
| CAGCCGCATGGTGGTGTCAATATTT | 221 |
| CCGCATGGTGGTGTCAATATTTGAT | 222 |

TABLE 17
19-nt siRNA targeted sequences identified
in ICT-1021.

| TARGET SEQUENCE | SEQ ID NO: |
|---|---|
| GCAATACCCAATTTCAATT | 223 |
| GAATCTTCCAAAGCGCAAA | 224 |
| TCTTCCAAAGCGCAAAGAA | 225 |
| TCCAAAGCGCAAAGAAGTT | 226 |
| CCAAAGCGCAAAGAAGTTA | 227 |
| GCAAAGAAGTTATTTGCCG | 228 |
| GAAGTTATTTGCCGAGGAT | 229 |
| TCATTCTCCTTCAAGGGAA | 230 |
| GCTTGGAGTTTGTCATCCT | 231 |
| TCATCCTACACCAACCTAA | 232 |

TABLE 18
25-nt siRNA targeted sequences identified
in ICT-1021.

| TARGET SEQUENCE | SEQ ID NO: |
|---|---|
| TCTACATTCCAAGGAGAGATTTAAA | 233 |
| CACCATGAATCTTCCAAAGCGCAAA | 234 |
| CGCAAAGAAGTTATTTGCCGAGGAT | 235 |
| AGAAGTTATTTGCCGAGGATCTGAT | 236 |
| ACAATATCATTCTCCTTCAAGGGAA | 237 |
| CAATATCATTCTCCTTCAAGGGAAT | 238 |
| CAAATGTGTTGTTGAAGCTATTTCT | 239 |
| GCTTGGAGTTTGTCATCCTACACCA | 240 |
| AGTTTGTCATCCTACACCAACCTAA | 241 |
| CATCCTACACCAACCTAATTCAAAT | 242 |

### Example 2

### Inhibition of Tumor Growth by ICT-1053 siRNA

MDA-MB-435 human breast carcinoma cells (ATCC, Manassas, Va.) were maintained in RPMI 1640 media (Sigma-Aldrich, St. Louis, Mo.) with 10% fetal bovine serum (FBS) (20 ml for one T-75 flask) at 37° C. and 5% CO₂. 4×10⁵ MDA-MB-435 cells in 50 μl OPTI-MEM (Invitrogen, Carlsbad, Calif.) were injected into fat pads under the nipples of mice on day 0 to induce tumors.

On Day 11 and Day 18, the mice were treated with either 10 ug ICT-1053 siRNA (5 ug of ICT-1053-siRNA-a mixed with 5 ug of ICT-1053-siRNA-b) or a negative control of 10 ug non-specific siRNA (NC) in 20 ul of PBS.

The ICT-1053 siRNA-a duplex consists of two complementary polynucleotide strands having the following sequences:

| | |
|---|---|
| r(UCUGUCUGCAGCCCAGACA)d(TT) | (SEQ ID NO: 73) |
| and | |
| r(UGUCUGGGCUGCAGACAGA)d(TT). | (SEQ ID NO: 74) |

The ICT-1053 siRNA-b duplex consists of two complementary polynucleotide strands having the following the sequences:

| | |
|---|---|
| r(GCGUGGAAGUUAACUUCAC)d(TT) | (SEQ ID NO: 75) |
| and | |
| r(GUGAAGUUAACUUCCACGC)d(TT). | (SEQ ID NO: 76) |

As a positive control, the tumors were treated with two VEGF siRNA inhibitors. The VEGF-siRNA-a duplex consists of two complementary polynucleotides having the following sequences:

| | |
|---|---|
| r(UCGAGACCCUGGUGGACAU)d(TT) | (SEQ ID NO: 77) |
| and | |
| r(AUGUCCACCAGGGUCUCGA)d(TT). | (SEQ ID NO: 78) |

The VEGF-siRNA-b duplex consists of two complementary polynucleotide having the following sequences:

| | |
|---|---|
| r(GGCCAGCACAUAGGAGAGA)d(TT) | (SEQ ID NO: 79) |
| and | |
| r(UCUCUCCUAUGUGCUGGCC)d(TT). | (SEQ ID NO: 80) |

As a negative control (NC-siRNA), the tumors were injected with two green fluorescent protein (GFP)-siRNA duplexes that have no homology with any human or mouse gene sequences. The GFP-siRNA-a duplex and the GFP-siRNA-b duplex sequences are given below in Example 5 (SEQ ID NOS:85-88).

The siRNA duplexes were transfected directly into the tumor xenografts using electroporation. Tumor size was monitored by measuring the length and width using an external caliper before every siRNA delivery and twice a week after the last siRNA delivery until the end point of experiment. Tumor volume is calculated as

Volume=width²×length×0.52.

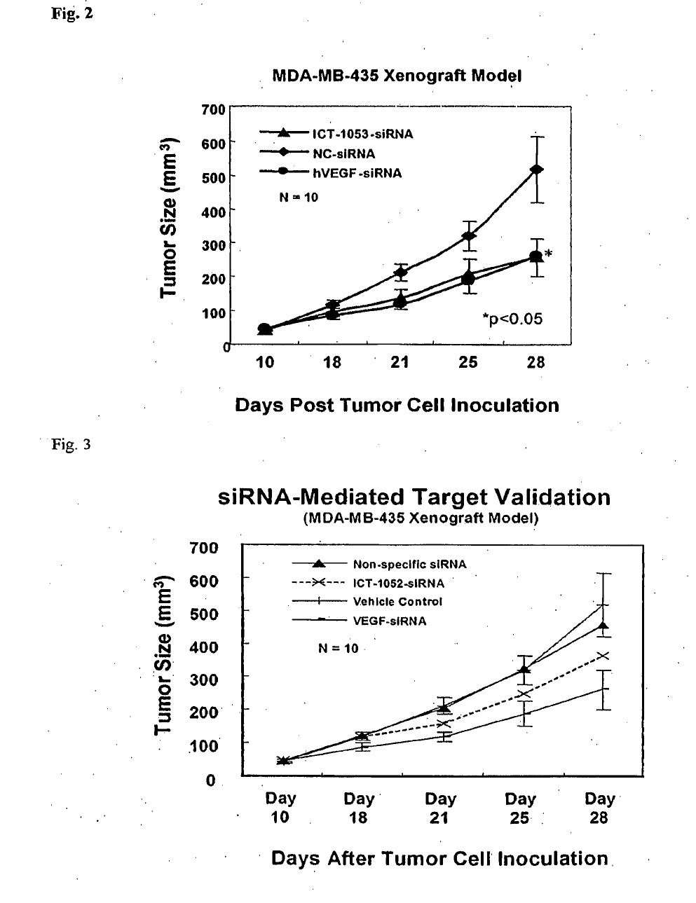

The results are shown in FIG. 2. The tumor size obtained upon treatment with the ICT-1053 siRNA is much smaller than that found with the nonspecific siRNA, and is essentially indistinguishable from the tumor size obtained with the VEGF siRNA positive control. This shows that siRNA targeting ICT-1053, which knocks down PDCD10 expression, powerfully limits the growth of tumors produced by MDA-MB-435 xenografts.

### Example 3

### Inhibition of Tumor Growth by ICT-1052 siRNA

In general, similar experimental procedures were used as described for Example 2. In the present Example control animals were treated with 1×PBS only (Vehicle Control in FIG. 3). On Day 11 and Day 18, the mice were treated with either 10 ug ICT-1052 siRNA (5 ug of ICT-1052-siRNA-a mixed with 5 ug of ICT-1052-siRNA-b) or 10 ug non-specific siRNA (NC) in 20 ul of PBS.

The ICT-1052 siRNA-a duplex consists of two complementary polynucleotide strands having the following sequences:

| | |
|---|---|
| r(CACCCAUCCAGAAUGUCAU)d(TT) | (SEQ ID NO: 81) |
| and | |
| r(AUGACAUUCUGGAUGGGUG)d(TT). | (SEQ ID NO: 82) |

The ICT-1052 siRNA-b duplex consists of two complementary polynucleotide strands having the following sequences:

| | |
|---|---|
| r(GCCAAUUUAUCAGGAGGUG)d(TT) | (SEQ ID NO: 83) |
| and | |
| r(CACCUCCUGAUAAAUUGGC)d(TT). | (SEQ ID NO: 84) |

The VEGF-siRNA-a and the VEGF-siRNA-b duplexes used as the positive control are the same as employed in Example 2 (SEQ ID NOS:77-80).

The results are shown in FIG. 3. It is seen that the ICT-1052 siRNA used in this Example reduces the tumor size in comparison to the negative controls, the PBS vehicle and the nonspecific siRNA (NC-siRNA). The beneficial effect of ICT-1052 siRNA was not as effective as VEGF si RNA. These results show that the ICT-1052 siRNA used in this Example, which knocks down c-Met expression, is effective to inhibit tumor growth of MDA-MB-435 xenografts.

### Example 4

### Inhibition of Tumor Growth by ICT-1052 siRNA

A549 human lung carcinoma cells (ATCC, Manassas, Va.) were maintained in DMEM media with 10% fetal bovine serum (FBS) at 37° C. and 5% CO₂. At Day 0, 1×10⁷ A549 cells in 100 ul DMEM medium without serum were inoculated s.c. into the back flank of anesthetized nude mice. At Day 6, the size of tumor was measured and animals were randomly assigned to treatment groups.

At Day 7, each tumor was transfected intratumorally with either 10 ug ICT-1052 siRNA (5 ug of ICT-1052-siRNA-a mixed with 5 ug of ICT-1052-siRNA-b (SEQ ID NOS:81-84); see Example 3) or 10 ug non-specific siRNA (NC) in 20 ul of PBS using an electroporation enhanced transfection procedure. Four more siRNA deliveries were carried out at Day 12, Day 16, Day 20, and Day 27. The tumor sizes were measured before each siRNA delivery, and twice a week after the last siRNA delivery.

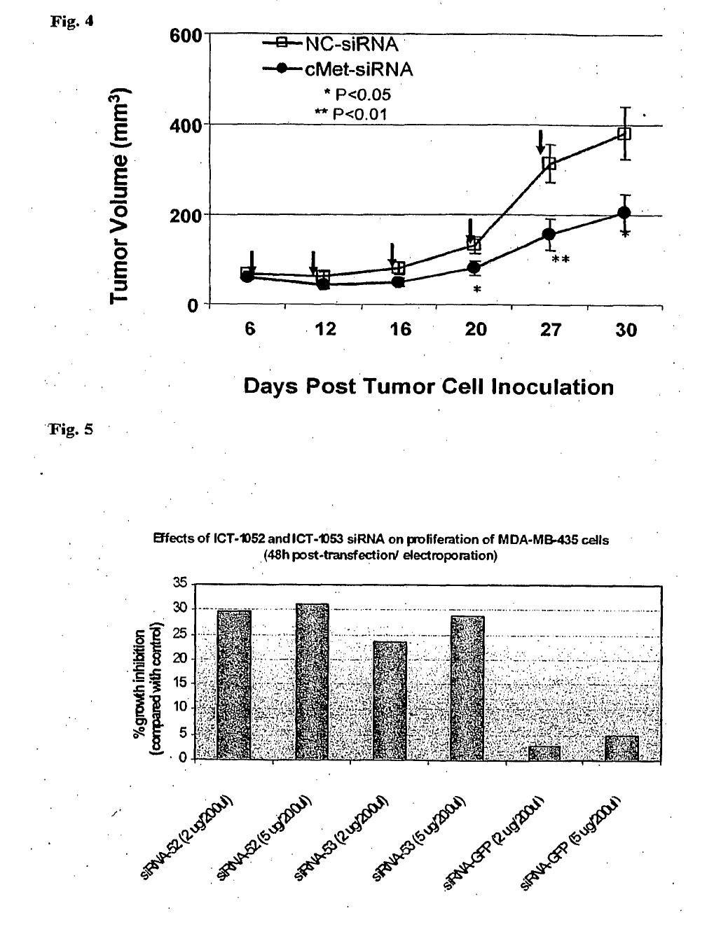

The results are shown in FIG. 4. It was observed that the treatment of A549 tumor with ICT-1052 siRNA in this Example significantly inhibits the growth rate of A549 xenografts compared to tumors treated with non-specific siRNA. These results show that the ICT-1052 siRNA used in this Example, which knocks down c-Met expression in the tumor, effectively inhibit the growth of A549 lung tumor.

### Example 5

### Inhibition of Tumor Growth by ICT-1052 siRNA and ICT-1053 siRNA

MDA-MB-435 human breast carcinoma cells were maintained in RPMI 1640 media with 10% FBS at 37° C. and 5% CO₂. The cells were transfected with the same ICT-1053 siRNAs used in Example 2 (SEQ ID NOS:73-76), or with the ICT-1052 siRNA used in Example 3 (SEQ ID NOS:81-84), at concentrations of 2 ug siRNA/2×10⁶ cells/200 ul DMEM medium or 5 ug siRNA/2×10⁶ cells/200 ul DMEM medium, using an electroporation mediated transfection method. In the control group, the cells were transfected with siRNA targeting green fluorescent protein reporter gene (GFP) at the same concentrations using an electroporation mediated transfection method. The GFP siRNA is a mixture of equal amount of GFP-siRNA-a duplex and GFP-siRNA-b duplex.

The GFP-siRNA-a duplex consists of two complementary polynucleotides with the following sequences:

| | |
|---|---|
| r(GCTGACCCTGAAGTTCATC)d(TT) | (SEQ ID NO: 85) |
| and | |
| r(GAUGAACUUCAGGGUCAGC)d(TT). | (SEQ ID NO: 86) |

The GFP-siRNA-b duplex consists of two complementary polynucleotides with following sequences:

| | |
|---|---|
| r(GCAGCACGACUUCUUCAAG)d(TT) | (SEQ ID NO: 87) |
| and | |
| r(CUUGAAGAAGUCGUGCUGC)d(TT). | (SEQ ID NO: 88) |

At 48 hours post transfection, the cell proliferation activity is measured using a Cell Proliferation Kit I (MTT-based, where MTT is 3-[4,5-Dimethylthiazol-2-yl]-2,5-diphenyltetrazolium bromide, or thiazolyl blue) (Roche Diagnostics, Indianapolis, Ind.). The medium in each well is aspirated, then 2 ml of serum-free DMEM is added into each well. 200 uL of MTT stock solution (MTT stock solution: 50 mg MTT in 10 mL PBS) is added to each well. The plate is incubated at 37° C. in CO₂ incubator for 3 hr. During this incubation period, viable cells convert MTT to a water-insoluble formazan dye. The medium in each well is removed, but not any of the formazan crystals. 2 mL of acidic isopropyl alcohol (500 mL isopropyl alcohol+3.5 mL of 6 N HCl) is added to each well, and the crystals are completely dissolved for about 10 min. 100 ul from each well are transferred into a 96-well plate, and the absorbance at 570 nm was read with background subtraction at 650 nm using a Microplate Reader (Model 680, Bio-Rad, Hercules, Calif.).

The results are shown in FIG. 5. It is seen that ICT-1052 siRNA and ICT-1053 siRNA provide 25-30% inhibition of growth of the MDA-MB-435 cells at both doses applied, whereas the control samples produce only 5% or less inhibition of growth. These data show that the targeting siRNAs employed in this study are effective to inhibit the growth of human breast carcinoma cells in culture.

### Example 6

### Inhibition of Proliferation of Cancer Cells by ICT-1052 siRNA and ICT-1053 siRNA

HCT116 human colorectal carcinoma cells were maintained in DMEM media with 2.5% FBS at 37° C. and 5% CO₂. The HCT116 cells were transfected with the same ICT-1053 siRNA as used in Example 2 (SEQ ID NOS:73-76), or with the ICT-1052 siRNA used in Example 3 (SEQ ID NOS:81-84), at a concentration of 5 ug siRNA/2×10⁶ cells/200 ul DMEM medium using an electroporation mediated transfection method. In the control group, the cells were transfected with NC-siRNA at the same concentration.

At 72 hours post transfection, the cell proliferation activity in the transfected HCT116 cells was measured using a Cell Proliferation Kit I (Roche Diagnostics, Indianapolis, Ind.) as described in Example 5.

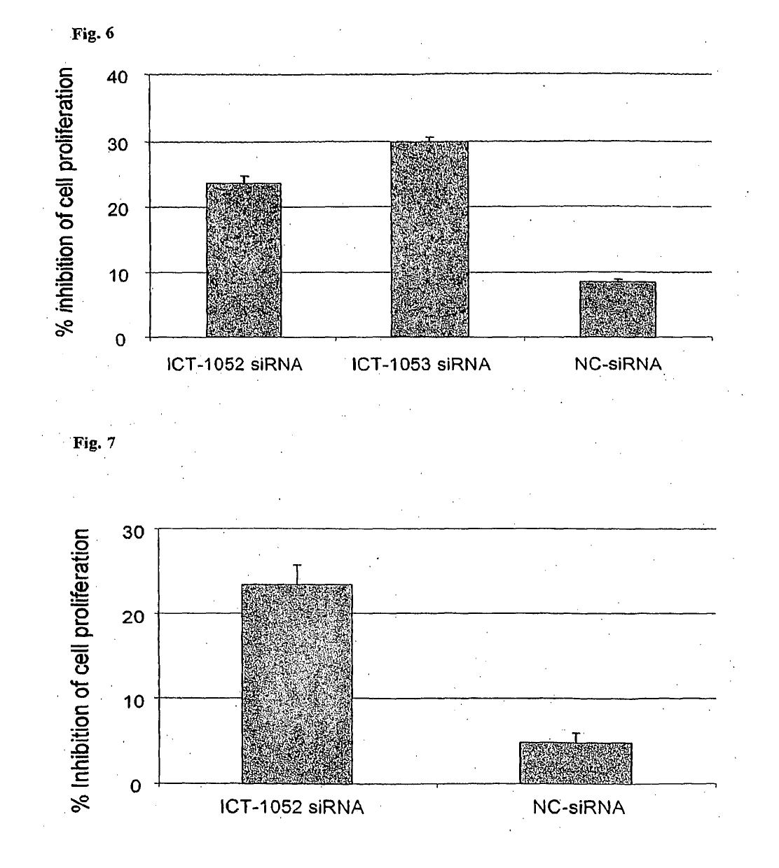

The results are shown in FIG. 6. It was observed that treatment of ICT-1052 siRNA or ICT-1053 siRNA resulted in 25-30% inhibition of proliferation of HCT116 cells, whereas the NC-siRNA treatment resulted in only 8% cell proliferation inhibition, compared to control cells that received mock treatment. These data demonstrate that the ICT-1052 and ICT-1053 siRNAs are effective inhibitors of the cell proliferation of human colon carcinoma cells in culture.

### Example 7

### Inhibition of Proliferation of Lung Carcinoma Cells by ICT-1052

A549 human lung carcinoma cells (ATCC, Manassas, Va.) were maintained in DMEM media with 10% fetal bovine serum (FBS) at 37° C. and 5% CO₂. The A549 cells were transfected with the same ICT-1052 siRNA as used in Example 3 (SEQ ID NOS:81-84) at a concentration of 5 ug siRNA/2×10⁶ cells/200 ul DMEM medium, using an electroporation mediated transfection method. In the control group, the A549 cells were transfected with NC-siRNA as described in Example 2 (SEQ ID NOS:85-88). At 72 hours post transfection, the cell proliferation activity in the transfected cells was measured, using a Cell Proliferation Kit I (Roche Diagnostics, Indianapolis, Ind.) as described in Example 5.

The results are shown in FIG. 7. It was observed that treatment of ICT-1052 siRNA resulted in an about 25% cell proliferation inhibition of A549 cells, whereas the NC-siRNA treatment resulted in only a 5% or less cell proliferation inhibition, compared to control cells that received mock treatment. These data demonstrate that the ICT-1052 siRNA can effectively inhibit the proliferation of human lung carcinoma cells in culture.

### Example 8

### Inhibition of Tumor Growth by ICT-1027 siRNA

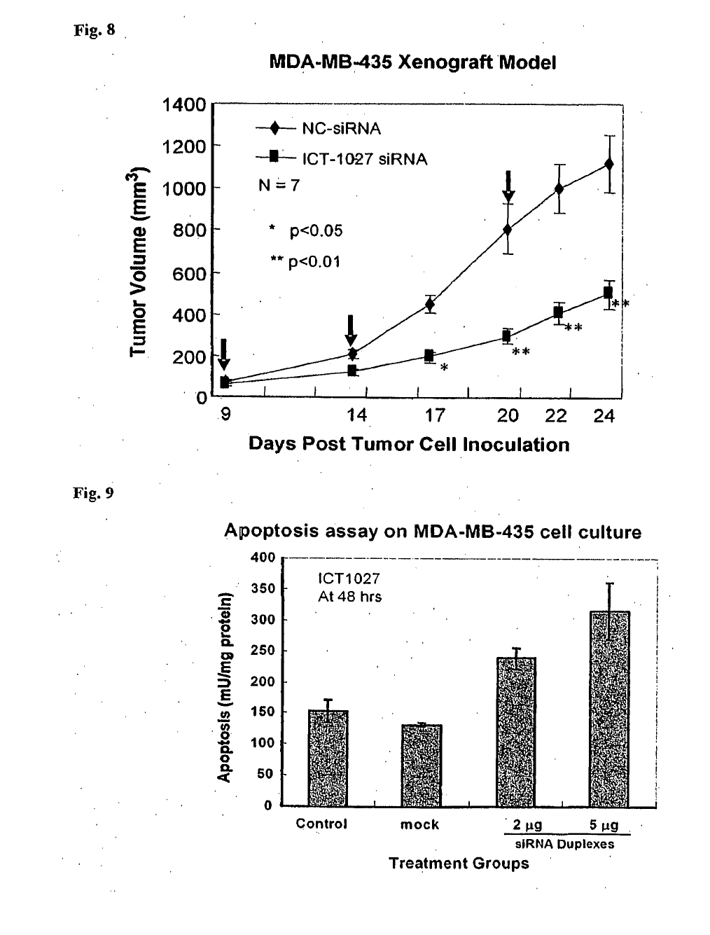

A similar experimental procedure was used as in Examples 2 and 3, with the modification that the siRNA was administrated at Day 9, Day 14, and Day 20 (indicated with arrows in FIG. 8). The mice were treated with either 10 ug ICT-1027 siRNA (5 ug of ICT-1027-siRNA-a mixed with 5 ug of ICT-1027-siRNA-b) or 10 ug GFP-siRNA in 20 ul of PBS.

The ICT-1027 siRNA-a duplex consists of two complementary polynucleotide strands having the following sequences:

| | |
|---|---|
| r(GGGGGGACAUCCUCAAGGU)d(TT) | (SEQ ID NO: 89) |
| and | |
| r(ACCUUGAGGAUGUCCCCCC)d(TT). | (SEQ ID NO: 90) |

The ICT-1027 siRNA-b duplex consists of two complementary polynucleotide strands having the following sequences:

| | |
|---|---|
| r(UCCCCAGAGCCAAGGCAGA)d(TT) | (SEQ ID NO: 91) |
| and | |
| r(UCUGCCUUGGCUCUGGGGA)d(TT). | (SEQ ID NO: 92) |

GFP siRNA serves as a negative control, and is a mixture of equal amount of GFP-siRNA-a duplex and GFP-siRNA-b duplex (SEQ ID NOS:85-88), as described in Example 2 4. The siRNA duplexes were intratumorally injected into the tumor xenograft.

The results are presented in FIG. 8. It is seen that the ICT-1027 siRNA mixture, which knocks down Grb2 expression, significantly inhibits solid tumor growth of the MDA-MB-435 xenograft, compared to the GFP siRNA control.

### Example 9

### Promotion of Apoptosis of Tumor Cells by ICT-1027 siRNA

MDA-MB-435 human breast carcinoma cells were maintained in RPMI 1640 media with 10% FBS at 37° C. and 5% CO₂. The cells were transfected with ICT-1027 siRNA using the sequences described in Example 8 (SEQ ID NOS:89-92) at concentrations of 2 ug siRNA/2×10⁶ cells/200 ul DMEM medium, or 5 ug siRNA/2×10⁶ cells/200 ul DMEM medium, using an electroporation mediated transfection method. In the control group, the cells did not received any treatment. In the mock group, the cells were treated with the same electroporation procedure but without siRNA in the medium. At 48 hours post transfection, the apoptosis activity in the cells was measured by quantitative determination of cytoplasmic histone-DNA fragments, which are indicative of apoptosis, using a Cell Death Detection ELISA kit (Roche Diagnostics). The assay is based on a quantitative sandwich-enzyme-immunoassay principle using mouse monoclonal antibodies directed against DNA and histones, respectively, which allows the specific determination of mono- and oligonucleosomes in the cytoplasmic fraction of cell lysates. Cells in each well are lysed with lysis buffer provided with the kit. 20 ul cell lysate from each well is transferred into streptavidin-coated microtiter plates and incubated with mouse monoclonal antihistone-biotin antibody and mouse monoclonal anti-DNA-peroxidase. Unbound antibodies are washed out. The amount of nucleosome is determined quantitatively by evaluating peroxidase activity photometrically with 2,2′-azino-bis-(3-ethylbenzthiazoline-6-sulfonic acid; ABTS) as substrate. The plate is then placed on a plate reader and the absorbance is measured at 590 nm using a Microplate Reader (Model 680, Bio-Rad, Hercules, Calif.).

The results are shown in FIG. 9. It is seen that, compared to the control and mock samples, ICT-1027 siRNA, which knocks down Grb2 gene expression; induces significant apoptosis in a dose-dependent fashion. The results suggest that inhibitory RNA directed against ICT-1027 inhibits tumor growth of MDA-MB-435 xenografts (Example 8) by inducing apoptosis of tumor cells.

### Example 10

### Inhibition of Growth of a Breast Cancer Xenografts by ICT-1051 siRNA

A similar experimental procedure as described in Example 2 was used in this Example to validate ICT-1051 (A-Raf) as a target.

On Day 11, and Day 18, the MDA-MB-435 tumor were treated with either 10 ug ICT-1051 siRNA (5 ug of ICT-1051-siRNA-a mixed with 5 ug of ICT-1051-siRNA-b) or 10 ug NC-siRNA.

The ICT-1051 siRNA-a duplex consists of two complementary polynucleotide with following sequences:

| | |
|---|---|
| r(GAGUUACCUUCCUAAUGCA)d(TT) | (SEQ ID NO: 93) |
| and | |
| r(UGCAUUAGGAAGGUAACUC)d(TT). | (SEQ ID NO: 94) |

The ICT-1051 siRNA-b duplex consists of two complementary polynucleotide with following sequences:

| | |
|---|---|
| r(GAUUCCCUUGGUAUAUUCA)d(TT) | (SEQ ID NO: 95) |
| and | |
| r(UGAAUAUACCAAGGGAAUC)d(TT). | (SEQ ID NO: 96) |

NC-siRNA serves as a negative control, and is a mixture of equal amount of GFP-siRNA-a duplex and GFP-siRNA-b duplex, as described in Example 2.

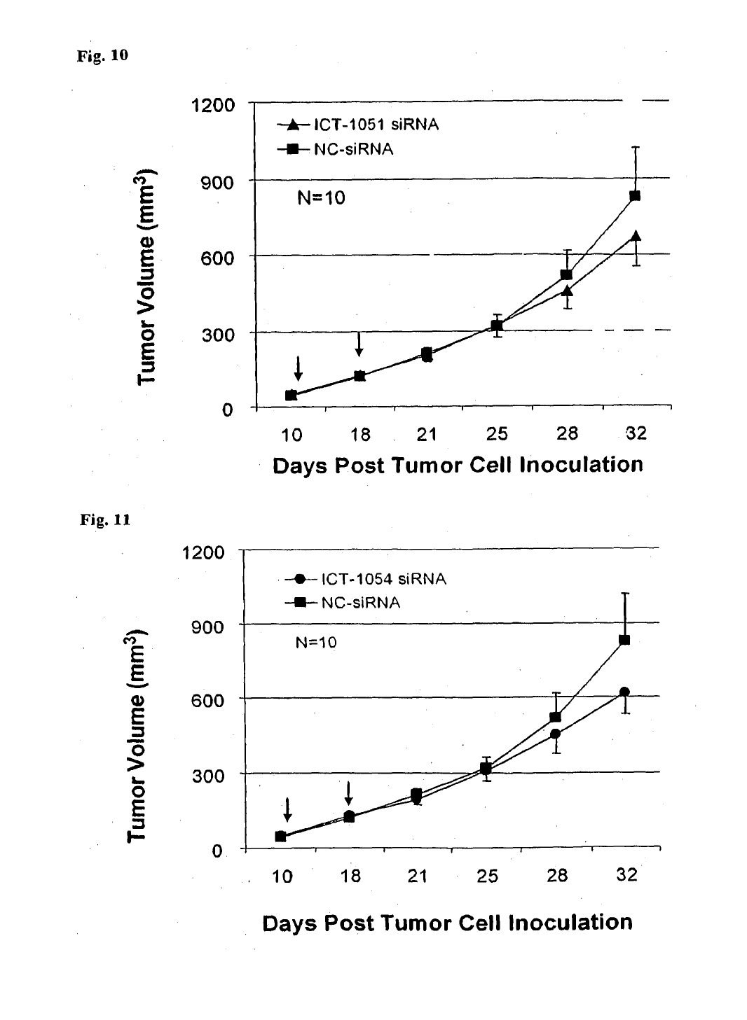

The results are presented in FIG. 10. The ICT-1051 siRNA mixture, which knocks down A-Raf expression, slowed down the growth of the MDA-MB-435 xenograft, compared to the NC-siRNA treated xenografts.

### Example 11

### Inhibition of Growth Breast Cancer Xenografts by ICT-1054 siRNA

A similar experimental procedure as described in Example 2 was used in this Example to validate ICT-1054 (PCDP6) as a target for cancer therapy.

On Day 11, and Day 18, the MDA-MB-435 tumor were treated with either 10 ug ICT-1054 siRNA (5 ug of ICT-1054-siRNA-a mixed with 5 ug of ICT-1054-siRNA-b) or 10 ug NC-siRNA.

The ICT-1054 siRNA-a duplex consists of two complementary polynucleotide with following sequences:

| | |
|---|---|
| r(GACAGGAGUGGAGUGAUAU)d(TT) | (SEQ ID NO: 97) |
| and | |
| r(AUAUCACUCCACUCCUGUC)d(TT). | (SEQ ID NO: 98) |

The ICT-1054 siRNA-b duplex consists of two complementary polynucleotide with following sequences:

| | |
|---|---|
| r(CUUCAGCGAGUUCACGGGU)d(TT) | (SEQ ID NO: 99) |
| and | |
| r(ACCCGUGAACUCGCUGAAG)d(TT). | (SEQ ID NO: 100) |

NC-siRNA serves as a negative control, and is a mixture of equal amount of GFP-siRNA-a duplex and GFP-siRNA-b duplex, as described in Example 2.

The results are presented in FIG. 11. The ICT-1054 siRNA mixture, which knocks down PCDP6 expression, slowed down the growth of the MDA-MB-435 xenograft, compared to the NC-siRNA treated xenografts.

### Example 12

### Inhibition of Growth of Breast Cancer Xenografts by ICT-1020

A similar experimental procedure as described in Example 2 was used in this Example to validate ICT-1020 (Dicer) as a target for cancer therapy, with modified schedule for siRNA administration.

In this Example, the MDA-MB-435 tumor xenografts were treated on Day 9 and Day 14 with either 10 ug ICT-1020 siRNA (5 ug of ICT-1020-siRNA-a mixed with 5 ug of ICT-1020-siRNA-b) or 10 ug NC-siRNA.

The ICT-1020 siRNA-a duplex consists of two complementary polynucleotide with following sequences:

| | |
|---|---|
| r(UGGGUCCUUUCUUUGGACU)d(TT) | (SEQ ID NO: 101) |
| and | |
| r(AGUCCAAAGAAAGGACCCA)d(TT). | (SEQ ID NO: 102) |

The ICT-1020 siRNA-b duplex consists of two complementary polynucleotide with following sequences:

| | |
|---|---|
| r(CUGCUUGAAGCAGCUCUGG)d(TT) | (SEQ ID NO: 103) |
| and | |
| r(CCAGAGCUGCUUCAAGCAG)d(TT). | (SEQ ID NO: 104) |

NC-siRNA serves as a negative control, and is a mixture of equal amount of GFP-siRNA-a duplex and GFP-siRNA-b duplex, as described in Example 2.

The results are presented in FIG. 12. The ICT-1020 siRNA treatment, which specifically knocks down Dicer expression within tumor cells, significantly reduced the growth rate of the MDA-MB-435 xenograft, compared to the xenografts treated with the negative control NC-siRNA.

### Example 13

### Inhibition of Growth of Breast Cancer Xenografts by ICT-1021 siRNA

A similar experimental procedure as described in Example 2 was used in this Example to validate ICT-1021 (MD2 protein) as a target for cancer therapy. On Day 11 and Day 18, the MDA-MB-435 tumors were treated with either 10 ug ICT-1021 siRNA (5 ug of ICT-1021-siRNA-a mixed with 5 ug of ICT-1021-siRNA-b) or 10 ug NC-siRNA.

The ICT-1021 siRNA-a duplex consists of two complementary polynucleotide with following sequences:

| | |
|---|---|
| r(GCUCAGAAGCAGUAUUGGG)d(TT) | (SEQ ID NO: 105) |
| and | |
| r(CCCAAUACUGCUUCUGAGC)d(TT). | (SEQ ID NO: 106) |

The ICT-1021 siRNA-b duplex consists of two complementary polynucleotide with following sequences:

| | |
|---|---|
| r(UGCAAUACCCAAUUUCAAU)d(TT) | (SEQ ID NO: 107) |
| and | |
| r(AUUGAAAUUGGGUAUUGCA)d(TT). | (SEQ ID NO: 108) |

NC-siRNA serves as a negative control, and is a mixture of equal amount of GFP-siRNA-a duplex and GFP-siRNA-b duplex, as described in Example 2.

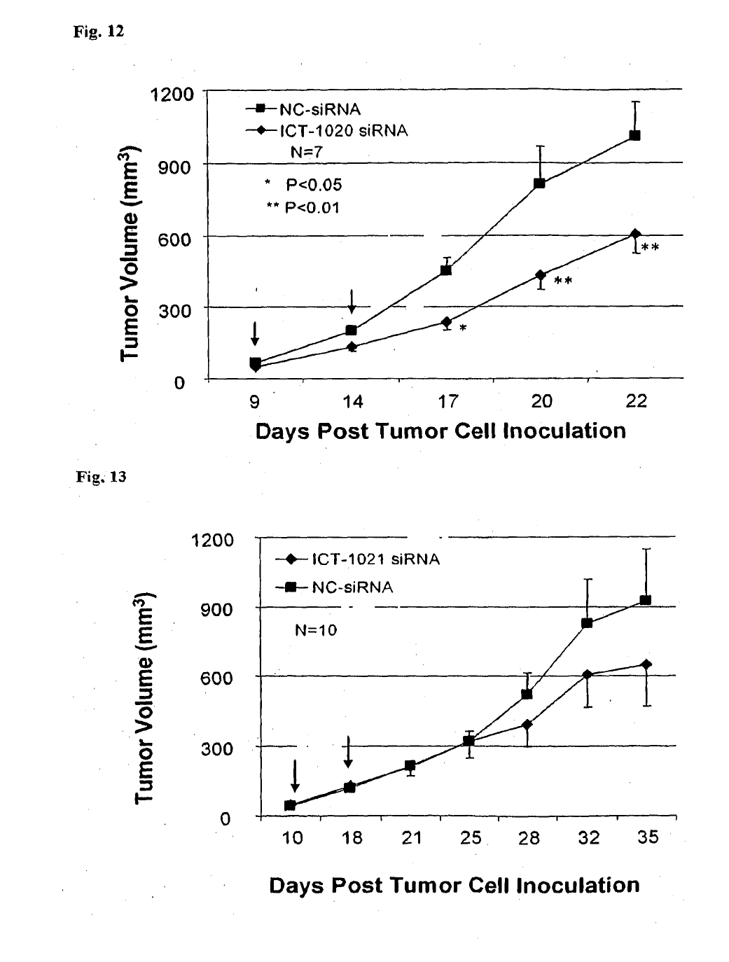

The results are presented in FIG. 13. The ICT-1021 siRNA treatment, which specifically knocks down MD2 protein expression within tumor cells, reduced the growth rate of the MDA-MB-435 xenograft, compared to the NC-siRNA treated xenografts.

### Example 14

### Inhibition of Growth of Breast Cancer Xenografts by ICT-1022 siRNA

A similar experimental procedure as described in Example 2 was used in this Example to validate ICT-1022 (GAGE-2) as a target for cancer therapy. In this Example the MDA-MB-435 xenografts were treated on Day 10 and Day 15 with either 10 ug ICT-1022 siRNA (5 ug of ICT-1022-siRNA-a mixed with 5 ug of ICT-1022-siRNA-b) or 10 ug NC-siRNA.

The ICT-1022 siRNA-a duplex consists of two complementary polynucleotide with following sequences:

| | |
|---|---|
| r(UGAUUGGGCCUAUGCGGCC)d(TT) | (SEQ ID NO: 109) |
| and | |
| r(GGCCGCAUAGGCCCAAUCA)d(TT). | (SEQ ID NO: 110) |

The ICT-1022 siRNA-b duplex consists of two complementary polynucleotide with following sequences:

| | |
|---|---|
| r(GUGGAACCAGCAACACCUG)d(TT) | (SEQ ID NO: 111) |
| and | |
| r(CAGGUGUUGCUGGUUCCAC)d(TT). | (SEQ ID NO: 112) |

NC-siRNA serves as a negative control, and is a mixture of equal amount of GFP siRNA-a duplex and GFP-siRNA-b duplex, as described in Example 2.

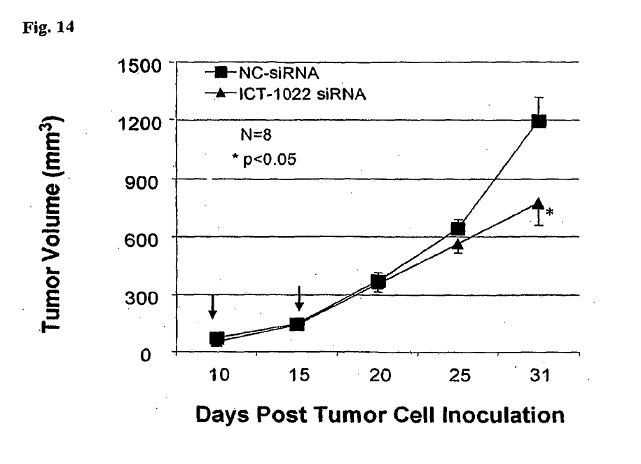

The growth curves of the siRNA treated MDA-MB-435 xenografts are presented in FIG. 14. The ICT-1022 siRNA treatment, which specifically knocks down GAGE-2 expression within tumor cells, significantly reduced the growth rate of the MDA-MB-435 xenograft, compared to the NC-siRNA treated xenografts.

Other embodiments and uses of the invention will be apparent to those skilled in the art from consideration of the specification and practice of the invention disclosed herein. All references and materials cited herein, including all U.S. and foreign patents and patent applications, are specifically and entirely hereby incorporated herein by reference. It is intended that the specification and examples be considered exemplary only, with the true scope and spirit of the invention indicated by the following claims.

### REFERENCES

1. Lu, Y. P., Xie, Y. F. and Woodle, M. C. (2003). siRNA-mediated antitumorgenesis for drug target validation and therapeutics. Current Opinion in Molecular Therapeutics. 5, 225-234.
2. Lu P., F. Xie, P. Scaria, and M. Woodle (2003). From Correlation to Causation to Control: Utilizing Preclinical Disease Models To Improve Cancer Target Discovery. Preclinica. 1:31-42.
3. Lu, P. Y. and M. Woodle (2005) Delivering siRNA in vivo For functional genomics can novel therapeutics. In RNA Interference Technology. Cambridge University Press P 303-317.
4. Lu, P. Y., et al., (2005). Modulation of angiogenesis with siRNA inhibitors for novel therapeutics. Trend in Molecular Medicine, In press.
5. Me, F. Y., et al., (2004). Delivering sirna to Animal Disease Models for Validation of Novel Drug Targets In Vivo. PharmaGenomics, July/August, 28-38.
6. Bottaro, D. P.; Rubin, J. S.; Faletto, D. L.; Chan, A. M.-L.; Kmiecik, T. E.; Vande Woude, G. F.; Aaronson, S. A. (1991): Identification of the hepatocyte growth factor receptor as the c-met proto-oncogene product. Science 251: 802-804.
7. Carrolo, M.; Giordano, S.; Cabrita-Santos, L.; Corso, S.; Vigario, A. M.; Silva, S.; Leiriao, P.; Carapau, D.; Armas-Portela, R.; Comoglio, P. M.; Rodriguez, A.; Mota, M. M. (2003): Hepatocyte growth factor and its receptor are required for malaria infection. Nature Med. 9: 1363-1369.
8. Cooper, C. S. (1992): The met oncogene: from detection by transfection to transmembrane receptor for hepatocyte growth factor. Oncogene 7: 3-7.
9. Cooper, C. S.; Park, M.; Blair, D. G.; Tainsky, M. A.; Huebner, K.; Croce, C. M.; Vande Woude, G. F. (1984): Molecular cloning of a new transforming gene from a chemically transformed human cell line. Nature 311: 29-33.
10. Dean, M.; Kozak, C.; Robbins, J.; Callahan, R.; O'Brien, S.; Vande Woude, G. F. (1987): Chromosomal localization of the met proto-oncogene in the mouse and cat genome. Genomics 1: 167-173.
11. Dean, M.; Park, M.; Le Beau, M. M.; Robins, T. S.; Diaz, M. O.; Rowley, J. D.; Blair, D. G.; Vande Woude, G. F. (1985): The human met oncogene is related to the tyrosine kinase oncogenes. Nature 318: 385-388.
12. Giordano, S.; Bardelli, k; Zhen, Z.; Menard, S.; Ponzetto, C.; Comoglio, P. M. (1997): A point mutation in the MET oncogene abrogates metastasis without affecting transformation. Proc. Nat. Acad. Sci. 94: 13868-13872.
13. Giordano, S.; Corso, S.; Conrotto, P.; Artigiani, S.; Gilestro, G.; Barberis, D.; Tamagnone, L.; Comoglio, P. M. (2002): The Semaphorin 4D receptor controls invasive growth by coupling with Met. Nature Cell Biol. 4: 720-724.
14. Jeffers, M.; Fiscella, M.; Webb, C. P.; Anver, M.; Koochekpour, S.; Vande Woude, G. F. (1998): The mutationally activated Met receptor mediates motility and metastasis. Proc. Nat. Acad. Sci. 95: 14417-14422.
15. Maina, F.; Casagranda, F.; Audero, E,; Simeone, A.; Comoglio, P. M.; Klein, R.; Ponzetto, C. (1996): Uncoupling of Grb2 from the Met receptor in vivo reveals complex roles in muscle development. Cell 87: 531-542.
16. Park, W. S.; Doug, S. M,; Kim, S. Y.; Na, E. Y.; Shin, M. S.; Pi, J. H.; Kim, B. J.; Bae, J. H.; Hong, Y. K.; Lee, K. S.; Lee, S. H.; Yoo, N. J.; Jang, J. J.; Pack, S.; Zhuang, Z.; Schmidt, L.; Zbar, B.; Lee, J. Y. (1999): Somatic mutations in the kinase domain of the Met/hepatocyte growth factor receptor gene in childhood hepatocellular carcinomas. Cancer Res. 59: 307-310.
17. Bergametti, F.; Denier, C.; Labauge, P.; Arnoult, M.; Boetto, S.; Clanet, M.; Coubes, P.; Echenne, B.; Ibrahim, R.; Irthum, B.; Jacquet, G.; Lonjon, M.; Moreau, J. J.; Neau, J. P.; Parker, F.; Tremoulet, M.; Tournier-Lasserve, E.; Societe Francaise de Neurochirurgie (2005): Mutations within the programmed cell death 10 gene cause cerebral cavernous malformations. Am. J. Hunt. Genet. 76: 42-51,
18. Bochmann, H.; Gehrisch, S.; Jaross, W. (1999): The gene structure of the human growth factor bound protein GRB2. Genomics 56: 203-207.
19. Cheng, A. M.; Saxton, T. M.; Sakai, R.; Kulkarni, S.; Mbamalu, G.; Vogel, W.; Tortorice, C. G.; Cardif R. D.; Cross, J. C.; Muller, W. J.; Pawson, T. (1998): Mammalian Grb2 regulates multiple steps in embryonic development and malignant transformation. Cell 95: 793-803.
20. He, Y.; Nakao, H.; Tan, S.-L.; Polyak, S. J.; Neddermann, P.; Vijaysri, S.; Jacobs, B. L.; Katze, M. G. (2002): Subversion of cell signaling pathways by hepatitis C virus nonstructural 5A protein via interaction with Grb2 and P85 phosphatidylinositol 3-kinase. J. Virol. 76: 9207-9217.
21. Hill, R. J.; Zozulya, S.; Lu, Y.-L.; Ward, K.; Gishizky, M.; Jallal, B. (2002): The lymphoid protein tyrosine phosphatase Lyp interacts with the adaptor molecule Grb2 and functions as a negative regulator of T-cell activation. Exp. Hemat. 30: 237-244.
22. Huebner, K.; Kastury, K.; Druck, T.; Salcini, A. E.; Lanfrancone, L.; Pelicci, G.; Lowenstein, E.; Li, W.; Park, S.-H.; Cannizzaro, L.; Pelicci, P. G.; Schlessinger, J. (1994) Chromosome locations of genes encoding human signal transduction adapter proteins, Nck (NCK), Shc (SHC1), and Grb2 (GRB2). Genomics 22: 281-287.
23. Matuoka, K.; Shibasaki, F.; Shibata, M.; Takenawa, T. (1993): Ash/Grb2, a SH2/SH3-containing protein, couples to signaling for mitogenesis and cytoskeletal reorganization by EGF and PDGF. EMBO J. 12: 3467-3473,
24. Yulug, I. G.; Egan, S. E.; See, C. G.; Fisher, E. M. C. (1994): Mapping GRB2, a signal transduction gene in the human and the mouse. Genomics 22: 313-318.
25. Lowenstein, E. J.; Daly, R. J.; Batzer, A. G.; Li, W.; Margolis, B.; Lammers, R.; Ullrich, A.; Skolnik, E. Y.; Bar-Sagi, D.; Schlessinger, J. (1992): The SH2 and SH3 domain-containing protein GRB2 links receptor tyrosine kinases to ras signaling. Cell 70: 431-442.
26. Cheng, A. M.; Saxton, T. M.; Sakai, R.; Kulkarni, S.; Mbamalu, G.; Vogel, W.; Tortorice, C. G.; Cardiff R. D.; Cross, J. C.; Muller, W. J.; Pawson, T. (1998): Mammalian Grb2 regulates multiple steps in embryonic development and malignant transformation. Cell 95: 793-803.

1. An isolated targeting polynucleotide whose length is 200 or fewer nucleotides, the polynucleotide comprising a first nucleotide sequence wherein the first nucleotide sequence targets an ICT-1053 gene, or an ICT-1052 gene, or an ICT-1027 gene, or an ICT-1051 gene, or an ICT-1054 gene, or an ICT-1020 gene, or an ICT-1021 gene, or an ICT-1022 gene, wherein any T (thymidine) or any U (uridine) may optionally be substituted by the other and wherein the first nucleotide sequence consists of
a) a sequence whose length is any number of nucleotides from 15 to 30, or
b) a complement of a sequence given in a).

2. (canceled)

3. The polynucleotide according to claim 1 wherein the first nucleotide sequence consists of
a) a sequence that targets a sequence chosen from SEQ ID NOS: 7-76, 81-84, and 89-242;
b) an extended sequence longer than, and comprising, the targeting sequence given in item a), wherein the extended sequence targets an ICT-1053 gene, or an ICT-1052 gene, or an ICT-1027 gene, or an ICT-1051 gene, or an ICT-1054 gene, or an ICT-1020 gene, or an ICT-1021 gene, or an ICT-1022 gene, and the targeting sequence targets a sequence chosen from SEQ ID NOS: 7-76, 81-84, and 89-242;
c) a fragment of a sequence that targets a sequence chosen from SEQ ID NOS:7-76, 81-84, and 89-242 wherein the fragment consists of a sequence of contiguous bases at least 15 nucleotides in length and at most one base shorter than the chosen sequence;
d) a targeting sequence wherein up to 5 nucleotides differ from a sequence that targets a sequence chosen from SEQ ID NOS:7-76, 81-84, and 89-242; or
e) a complement of a sequence given in a)-d).

4. The polynucleotide according to claim 1 wherein the length of the first nucleotide sequence is any number of nucleotides from 21 to 25.

5. The polynucleotide according to claim 1 consisting of a sequence chosen from SEQ ID NOS:7-76, 81-84, and 89-242, optionally including a dinucleotide overhang bound to the 3′ of the chosen sequence.

5a-9. (canceled)

10. A double stranded polynucleotide comprising a first targeting polynucleotide strand according to claim 1 and a second polynucleotide strand comprising a second nucleotide sequence that is substantially complementary to at least the first nucleotide sequence of the first polynucleotide strand and is hybridized thereto.

11-16. (canceled)

17. A pharmaceutical composition comprising the polynucleotide according to claim 1 and a pharmaceutically acceptable carrier.

18-20. (canceled)

21. A method of inhibiting the growth of a cancer cell in a subject, comprising the step of administering to the subject the pharmaceutical composition according to claim 17.

22. A method of promoting apoptosis in a cancer cell in a subject, comprising the step of administering to the subject the pharmaceutical composition according to claim 17.

23-31. (canceled)

32. A method for decreasing the expression of an ICT-1053 gene, an ICT-1052 gene, an ICT-1027 gene, an ICT-1051 gene, an ICT-1054 gene, an ICT-1020 gene, an ICT-1021 gene or an ICT-1022 gene in a cell, comprising introducing into the cell the nucleic acid molecule according to claim 1.

33. The polynucleotide according to claim 1, comprising at least one nucleotide that is modified.

34. The polynucleotide according to claim 33, wherein the at least one modified nucleotide comprises a modification in the phosphate group, the monosaccharide or the base.

35. The polynucleotide according to claim 33, wherein the at least one modified nucleotide is a nucleotide comprising a 2′-O-methyl ribose.

36. The polynucleotide according to claim 10, wherein the polynucleotide is blunt-ended and wherein the first and the second nucleotide strands are each 25 nucleotides in length.

37. The composition according to claim 17, further comprising one or more additional targeting polynucleotides that induce RNA interference and decrease the expression of a gene of interest.

38. The composition according to claim 17, further comprising a cationic copolypeptide.

39. The composition according to claim 38, wherein the cationic copolypeptide is a histidine-lysine copolypeptide.

40. The composition according to claim 17, further comprising polyethylene glycol.

41. The composition according to claim 17, further comprising a targeting ligand.

42. An antibody directed against an ICT-1053 gene, an ICT-1052 gene, an ICT-1027 gene, an ICT-1051 gene, an ICT-1054 gene, an ICT-1020 gene, an ICT-1021 gene or an ICT-1022 gene product polypeptide.

43. A method of treating a cancer, a tumor or a precancerous growth in a subject, comprising the step of administering to the subject the antibody according to claim 42.
